(19) United States
(12) Patent Application Publication
(10) Pub. No.: US 2024/0208934 A1
(43) Pub. Date: Jun. 27, 2024

(54) ULK1/2 INHIBITORS AND THEIR USE THEREOF

(71) Applicant: LifeArc, London (GB)

(72) Inventors: Martin AMBLER, London (GB); Edward Giles McIVER, London (GB); Chidochangu MPAMHANGA, London (GB); Simon OSBORNE, London (GB)

(21) Appl. No.: 18/285,788

(22) PCT Filed: Apr. 6, 2022
(86) PCT No.: PCT/IB2022/000182

§ 371 Date: Oct. 5, 2023

Related U.S. Application Data
(60) Provisional application No. 63/171,763, filed on Apr. 7, 2021.

(51) Int. Cl. C07D 401/14; A61K 31/506; A61K 31/5377; A61K 45/06; C07D 239/48; C07D 401/12; C07D 403/12; C07D 405/14

(52) U.S. Cl. CPC C07D 401/14; A61K 31/506; A61K 31/5377; A61K 45/06; C07D 239/48; C07D 401/12; C07D 403/12; C07D 405/14

(57) ABSTRACT

Described herein are compounds that are ULK1/2 inhibitors and their use in the treatment of disorders such as cancers.

### CROSS-REFERENCE

This application claims the benefit of U.S. Provisional Application Ser. No. 63/171,763 filed Apr. 7, 2021 which is hereby incorporated by reference in its entirety.

### FIELD OF THE DISCLOSURE

The disclosure relates to ULK1/2 inhibitors and their use in the treatment of cancer sensitive to ULK1/2 inhibition.

### BACKGROUND

Autophagy, the cell process of self-digestion, plays a role in maintaining energy homoeostasis and protein synthesis and causes degradation of long-lived proteins and damaged organelles, indicating that it plays a role in cancer, by both protecting against and promoting cell death. The autophagy-related gene (Atg) family, with more than 35 members, regulates multiple stages of the process. UNC-51-like kinase 1 (ULK1) has been demonstrated to mediate autophagy. Studies have indicated that inhibition of ULK1 promotes apoptosis and suppresses tumor growth and metastasis in cancers. Dower et al., Mol. Cancer Ther; 17(11), 2018, pp. 2366-2376; Martin et al., iScience; 8, 2018, pp. 74-84; Tompkins et al., Yale Journal of Biology and Medicine; 92, 2019, pp. 707-718; Lin et al., Cell Death and Disease; 10, 2019, p. 139.

There is therefore an urgent need for developing ULK1 inhibitors for the treatment of cancers in subjects, including humans.

### SUMMARY OF THE DISCLOSURE

The present disclosure provides novel ULK1/2 inhibitors, and their use in the treatment of cancers which are sensitive to ULK1/2 inhibition (e.g. CML).

Disclosed herein is a compound of Formula (I), or a pharmaceutically acceptable salt, solvate, or stereoisomer thereof:

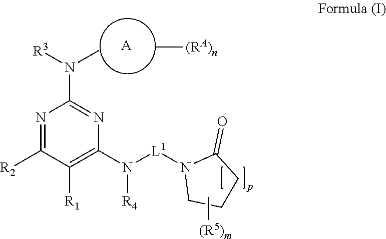

wherein:
R¹ is C₁-C₆haloalkyl or cycloalkyl;
R² is hydrogen, halogen, deuterium, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, or heterocycloalkyl;
R³ is hydrogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl;
R⁴ is hydrogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl;
each R⁵ is independently deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R⁵ on the same carbon are taken together to form an oxo;
Ring A is a 6- to 12-membered bicyclic ring optionally comprising 1-4 heteroatoms selected from the group consisting of O, S, N, P, and B;
each R^{A} is independently deuterium, halogen, —CN, —NO₂, —OH, —OR^{a}, —O(C₂-C₆alkylene)OR^{a}, —O(C₂-C₆alkylene)NR^{c}R^{d}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —NR^{b}C(═O)OR^{b}, —NR^{b}C(═O)NR^{c}R^{d}, —NHS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl are independently substituted with one, two, or three R^{Aa};
or two R^{A} on the same carbon are taken together to form an oxo;
each R^{Aa} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —NR^{b}C(═O)OR^{b}, —NR^{b}C(═O)NR^{c}R^{d}, —NHS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{Aa} on the same carbon are taken together to form an oxo;
L¹ is a C₃-C₄ alkylene optionally substituted with one, two, or three R^{L1};
each R^{L1} is independently deuterium, halogen, —CN, —NO₂, —OH, —OR^{a}, or —NR^{c}R^{d};
or two R^{L1} on the same carbon are taken together to form an oxo;
n is 1-4;
m is 0-4;
p is 1 or 2;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl;
or R^{c} and R^{d} are taken together with the atom to which they are attached to form a heterocycloalkyl optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl.

Disclosed herein is a compound of Formula (IIa) or (IIb), or a pharmaceutically acceptable salt, solvate, or stereoisomer thereof:

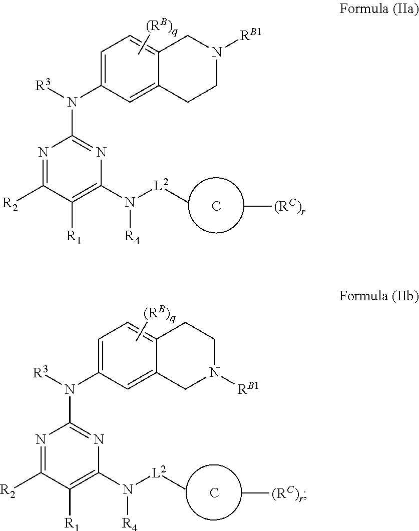

wherein:
R¹ is C₁-C₆haloalkyl or cycloalkyl;
R² is hydrogen, halogen, deuterium, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, or heterocycloalkyl;
R³ is hydrogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl;
R⁴ is hydrogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl;
each R^{B} is independently deuterium, halogen, —CN, —NO₂, —OH, —OR^{a}, —O(C₂-C₆alkylene)OR^{a}, —O(C₂-C₆alkylene)NR^{c}R^{d}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —NR^{b}C(═O)OR^{b}, —NR^{b}C(═O)NR^{c}R^{d}, —NHS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl are independently substituted with one, two, or three R^{Ba};
or two R^{B} on the same carbon are taken together to form an oxo;
each R^{Ba} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —NR^{b}C(═O)OR^{b}, —NR^{b}C(═O)NR^{c}R^{d}, —NHS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{Ba} on the same carbon are taken together to form an oxo;
R^{B1} is hydrogen, —S(═O)R^{a}, —S(═O)₂R^{a}, —S(═O)₂NR^{c}R^{d}, —C(═O)R^{a}, —C(═O)OR^{b}, —C(═O)NR^{C}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl);
Ring C is cycloalkyl, heterocycloalkyl, aryl, or heteroaryl;
each R^{C} is independently deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{C} on the same carbon are taken together to form an oxo;
L² is a C₁-C₄ alkylene optionally substituted with one, two, or three R^{L2};
each R^{L2} is independently deuterium, halogen, —CN, —NO₂, —OH, —OR^{a}, or —NR^{c}R^{d};
or two R^{L2} on the same carbon are taken together to form an oxo;
q is 0-4;
r is 0-4;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl;
or R^{c} and R^{d} are taken together with the atom to which they are attached to form a heterocycloalkyl optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl.

Disclosed herein is a compound of Formula (IIa), or a pharmaceutically acceptable salt, solvate, or stereoisomer thereof:

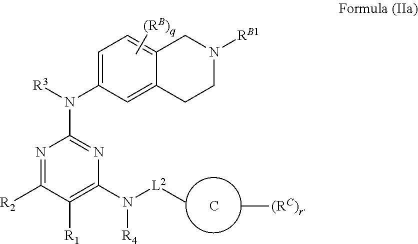

Disclosed herein is a compound of Formula (IIb), or a pharmaceutically acceptable salt, solvate, or stereoisomer thereof:

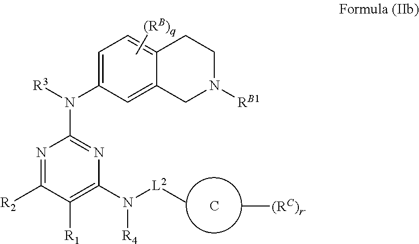

In accordance with a third aspect of the disclosure, there is provided a method of treating cancer sensitive to ULK1/2 inhibition in a subject in need thereof, comprising administering to the subject an effective amount of a compound of formula (I), (IIa), or (IIb), or a pharmaceutically acceptable salt, solvates, or stereoisomer thereof as defined above.

Additional embodiments of the disclosure also include methods for treating abnormal cell growth in a subject comprising administering to the subject a therapeutically effective amount of a compound as described herein or a pharmaceutically acceptable salt, solvate, or stereoisomer thereof. In certain such embodiments, the abnormal cell growth is cancer, and in certain of those embodiments the cancer is lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, or pituitary adenoma. Such cancers may be KRAS associated cancers. Of particular interest are cancers such as lung cancer, colon cancer, pancreatic cancer, and ovarian cancer. In some embodiment the method of treating cancer is a method of treating chronic myeloid leukaemia. In some embodiments, the cancer comprises a solid tumor.

In some embodiments, the cancer comprises a liquid tumor. In some embodiments, the cancer is chronic myeloid leukaemia.

In accordance with a fourth aspect of the disclosure there is provided the use of a compound of formula (I), (IIa), or (IIb), or a pharmaceutically acceptable salt, solvates, or stereoisomer thereof as defined above for the manufacture of a medicament for use in the treatment of cancer sensitive to ULK1/2 inhibition.

In some embodiments, the cancer in the subject includes those having one or more alterations in the MAPK pathway. Among the one or more alterations in the MAPK pathway, include cancers having alternations in one or more of the RAS, RAF, MEK, and ERK pathways. In some embodiments, the cancer in the subject has one or more alterations in the RAS pathway. In some embodiments, the cancer in the subject has one or more alterations in the RAF pathway. In some embodiments, the cancer in the subject has one or more alterations in the MEK pathway. In some embodiments, the cancer in the subject has one or more alterations in the ERK pathway.

In some embodiments, the subject is a mammalian subject. In a preferred embodiment, the subject is a human subject.

### DETAILED DESCRIPTION OF THE DISCLOSURE

“Abnormal cell growth”, as used herein, unless otherwise indicated, refers to cell growth that is independent of normal regulatory mechanisms (e.g., loss of contact inhibition). Abnormal cell growth may be benign (not cancerous), or malignant (cancerous). Abnormal cell growth includes the abnormal growth of: (1) tumor cells (tumors) that show increased expression of ULK1 or ULK2; (2) tumors that proliferate by aberrant ULK1 or ULK2 activation; and/or (3) tumors characterized by amplification or overexpression of the genes that express ULK1 or ULK2.

The term “additional anticancer agents” as used herein means any one or more therapeutic agent, other than a compound of the disclosure, that is or can be used in the treatment of cancer. In some embodiments, such additional anticancer agents include compounds derived from the following classes: mitotic inhibitors, alkylating agents, antimetabolites, antitumor antibiotics, anti-angiogenesis agents, topoisomerase I and II inhibitors, plant alkaloids, hormonal agents and antagonists, growth factor inhibitors, radiation, signal transduction inhibitors, such as inhibitors of protein tyrosine kinases and/or serine/threonine kinases, cell cycle inhibitors, biological response modifiers, enzyme inhibitors, antisense oligonucleotides or oligonucleotide derivatives, cytotoxics, immuno-oncology agents, and the like.

As used herein “cancer” refers to any malignant and/or invasive growth or tumor caused by abnormal cell growth. Cancer includes solid tumors named for the type of cells that form them, cancer of blood, bone marrow, or the lymphatic system. Examples of solid tumors include sarcomas and carcinomas.

Cancers of the blood include, but are not limited to, leukemia, lymphoma and myeloma. Cancer also includes primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of a different type from the latter one.

As used herein, the term “combination therapy” refers to the administration of a compound of the disclosure together with an at least one additional pharmaceutical or medicinal agent (e.g., one or more additional anticancer agents), either sequentially or simultaneously.

As used herein, “subject” refers to a human or animal subject. In certain preferred embodiments, the subject is a human.

The term “treat” or “treating” a cancer as used herein means to administer a compound of the present invention to a subject having cancer, or diagnosed with cancer, to achieve at least one positive therapeutic effect, such as, for example, reduced number of cancer cells, reduced tumor size, reduced rate of cancer cell infiltration into peripheral organs, or reduced rate of tumor metastases or tumor growth, reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

As used herein, a “pharmaceutically acceptable carrier” refers to a carrier or diluent that does not cause significant irritation to an organism and does not abrogate the biological activity and properties of the administered compound.

The terms below, as used herein, have the following meanings, unless indicated otherwise:

“Oxo” refers to ═O.

“Carboxyl” refers to —COOH.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, —COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, —COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, —COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aromatic ring, which may be monocyclic or bicyclic (for example, phenyl or naphthyl). In some embodiments, the aryl is a 6-membered aromatic ring (phenyl). Aryl radicals include, but are not limited to anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Aryl radicals include, but are not limited to 1,2,3,5,6,7-hexahydro-s-indacene, 2,3-dihydro-1H-indene, 1,2,3,4-tetrahydronaphthalene, 2,3,5,6,7,8-hexahydro-1H-cyclopenta[b]naphthalene, and 1,2,3,4,5,6,7,8-octahydroanthracene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, —COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic or polycyclic carbocyclic ring, which may include fused (when fused with an aryl or a heteroaryl ring, the cycloalkyl is bonded through a non-aromatic ring atom) or bridged ring systems. In some embodiments, the cycloalkyl is fully saturated.

Representative cycloalkyls include, but are not limited to, cycloalkyls having from three to fifteen carbon atoms (C₃-C₁₅ cycloalkyl or C₃-C₁₅ cycloalkenyl), from three to ten carbon atoms (C₃-C₁₀ cycloalkyl or C₃-C₁₀ cycloalkenyl), from three to eight carbon atoms (C₃-C₈ cycloalkyl or C₃-C₈ cycloalkenyl), from three to six carbon atoms (C₃-C₆ cycloalkyl or C₃-C₆ cycloalkenyl), from three to five carbon atoms (C₃-C₅ cycloalkyl or C₃-C₅ cycloalkenyl), or three to four carbon atoms (C₃-C₄ cycloalkyl or C₃-C₄ cycloalkenyl). In some embodiments, the cycloalkyl is a 3- to 10-membered cycloalkyl or a 3- to 10-membered cycloalkenyl. In some embodiments, the cycloalkyl is a 3- to 6-membered cycloalkyl or a 3- to 6-membered cycloalkenyl. In some embodiments, the cycloalkyl is a 5- to 6-membered cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbomyl, decalinyl, bicyclo[3.3.0] octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, —COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Deuteroalkyl” refers to an alkyl radical, as defined above, that is substituted by one or more deuteriums. In some embodiments, the alkyl is substituted with one deuterium. In some embodiments, the alkyl is substituted with one, two, or three deuteriums. In some embodiments, the alkyl is substituted with one, two, three, four, five, or six deuteriums. Deuteroalkyl include, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl or C₂-C₁₅ heterocycloalkenyl), from two to ten carbon atoms (C₂-C₁₀ heterocycloalkyl or C₂-C₁₀ heterocycloalkenyl), from two to eight carbon atoms (C₂-C₈ heterocycloalkyl or C₂-C₈ heterocycloalkenyl), from two to seven carbon atoms (C₂-C₇ heterocycloalkyl or C₂-C₇ heterocycloalkenyl), from two to six carbon atoms (C₂-C₆ heterocycloalkyl or C₂-C₆ heterocycloalkenyl), from two to five carbon atoms (C₂-C₅ heterocycloalkyl or C₂-C₅ heterocycloalkenyl), or two to four carbon atoms (C₂-C₄ heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl or a 3- to 8-membered heterocycloalkenyl. In some embodiments, the heterocycloalkyl is a 3- to 7-membered heterocycloalkyl or a 3- to 7-membered heterocycloalkenyl. In some embodiments, the heterocycloalkyl is a 3- to 6-membered heterocycloalkyl or a 3- to 6-membered heterocycloalkenyl. In some embodiments, the heterocycloalkyl is a 4- to 6-membered heterocycloalkyl or a 4- to 6-membered heterocycloalkenyl. In some embodiments, the heterocycloalkyl is a 5- to 6-membered heterocycloalkyl or a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, —COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

As used herein, a dashed bond, [symbol] or

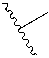

in a partial chemical structure, denotes the point of attachment of the partial chemical structure to the remainder of the compound of formula (I), (IIa), or (IIb).

### Compounds

Disclosed herein are compounds of Formula (I), or a pharmaceutically acceptable salt, solvate, or stereoisomer thereof:

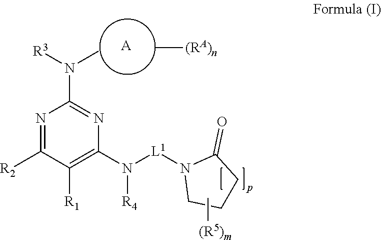

wherein:
R¹ is C₁-C₆haloalkyl or cycloalkyl;
R² is hydrogen, halogen, deuterium, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, or heterocycloalkyl;
R³ is hydrogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl;
R⁴ is hydrogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl;
each R⁵ is independently deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R⁵ on the same carbon are taken together to form an oxo;
Ring A is a 6- to 12-membered bicyclic ring optionally comprising 1-4 heteroatoms selected from the group consisting of O, S, N, P, and B;
each R^{A} is independently deuterium, halogen, —CN, —NO₂, —OH, —OR^{a}, —O(C₂-C₆alkylene)OR^{a}, —O(C₂-C₆alkylene)NR^{c}R^{d}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —NR^{b}C(═O)OR^{b}, —NR^{b}C(═O)NR^{c}R^{d}, —NHS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl are independently substituted with one, two, or three R^{Aa};
or two R^{A} on the same carbon are taken together to form an oxo;
each R^{Aa} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —NR^{b}C(═O)OR^{b}, —NR^{b}C(═O)NR^{c}R^{d}, —NHS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{Aa} on the same carbon are taken together to form an oxo;
L¹ is a C₃-C₄ alkylene optionally substituted with one, two, or three R^{L1};
each R^{L1} is independently deuterium, halogen, —CN, —NO₂, —OH, —OR^{a}, or —NR^{c}R^{d};
or two R^{L1} on the same carbon are taken together to form an oxo;
n is 1-4;
m is 0-4;
p is 1 or 2;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl;
or R^{c} and R^{d} are taken together with the atom to which they are attached to form a heterocycloalkyl optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl.

In some embodiments of a compound of Formula (I), or a pharmaceutically acceptable salt, solvate, or stereoisomer thereof, the compound of Formula (I) is not

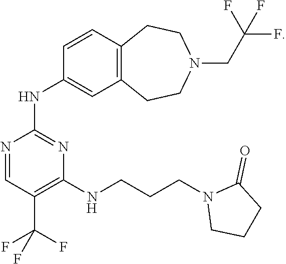

In some embodiments of a compound of Formula (I), or a pharmaceutically acceptable salt, solvate, or stereoisomer thereof, R² is hydrogen, halogen, deuterium, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl. In some embodiments of a compound of Formula (I), or a pharmaceutically acceptable salt, solvate, or stereoisomer thereof, R² is hydrogen, halogen, deuterium, C₁-C₆alkyl, C₁-C₆haloalkyl, or C₁-C₆deuteroalkyl.

In some embodiments of a compound of Formula (I), or a pharmaceutically acceptable salt, solvate, or stereoisomer thereof, R² is hydrogen, halogen, or C₁-C₆alkyl. In some embodiments of a compound of Formula (I), or a pharmaceutically acceptable salt, solvate, or stereoisomer thereof, R² is hydrogen.

In some embodiments of a compound of Formula (I), or a pharmaceutically acceptable salt, solvate, or stereoisomer thereof, R³ is hydrogen, C₁-C₆alkyl, or C₁-C₆haloalkyl. In some embodiments of a compound of Formula (I), or a pharmaceutically acceptable salt, solvate, or stereoisomer thereof, R³ is hydrogen or C₁-C₆alkyl. In some embodiments of a compound of Formula (I), or a pharmaceutically acceptable salt, solvate, or stereoisomer thereof, R³ is C₁-C₆alkyl. In some embodiments of a compound of Formula (I), or a pharmaceutically acceptable salt, solvate, or stereoisomer thereof, R³ is hydrogen.

In some embodiments of a compound of Formula (I), or a pharmaceutically acceptable salt, solvate, or stereoisomer thereof, R⁴ is hydrogen, C₁-C₆alkyl, or C₁-C₆haloalkyl. In some embodiments of a compound of Formula (I), or a pharmaceutically acceptable salt, solvate, or stereoisomer thereof, R⁴ is hydrogen or C₁-C₆alkyl. In some embodiments of a compound of Formula (I), or a pharmaceutically acceptable salt, solvate, or stereoisomer thereof, R⁴ is C₁-C₆alkyl. In some embodiments of a compound of Formula (I), or a pharmaceutically acceptable salt, solvate, or stereoisomer thereof, R⁴ is hydrogen.

In some embodiments of a compound of Formula (I), or a pharmaceutically acceptable salt, solvate, or stereoisomer thereof, R¹ is C₁-C₆haloalkyl. In some embodiments of a compound of Formula (I), or a pharmaceutically acceptable salt, solvate, or stereoisomer thereof, R¹ is CF₃. In some embodiments of a compound of Formula (I), or a pharmaceutically acceptable salt, solvate, or stereoisomer thereof, R¹ is cycloalkyl. In some embodiments of a compound of Formula (I), or a pharmaceutically acceptable salt, solvate, or stereoisomer thereof, R¹ is cyclopropyl.

In some embodiments of a compound of Formula (I), or a pharmaceutically acceptable salt, solvate, or stereoisomer thereof, L¹ is a C₃ alkylene optionally substituted with one, two, or three R^{L1}. In some embodiments of a compound of Formula (I), or a pharmaceutically acceptable salt, solvate, or stereoisomer thereof, L¹ is a C₄ alkylene optionally substituted with one, two, or three R^{L1}.

In some embodiments of a compound of Formula (I), or a pharmaceutically acceptable salt, solvate, or stereoisomer thereof, L¹ is C₃-C₄ alkylene. In some embodiments of a compound of Formula (I), or a pharmaceutically acceptable salt, solvate, or stereoisomer thereof, L¹ is C₃ alkylene. In some embodiments of a compound of Formula (I), or a pharmaceutically acceptable salt, solvate, or stereoisomer thereof, L¹ is C₄ alkylene.

In some embodiments of a compound of Formula (I), or a pharmaceutically acceptable salt, solvate, or stereoisomer thereof, each R^{L1} is independently deuterium, halogen, —OH, —OR^{a}, or —NR^{c}R^{d}; or two R^{L1} on the same carbon are taken together to form an oxo. In some embodiments of a compound of Formula (I), or a pharmaceutically acceptable salt, solvate, or stereoisomer thereof, each R^{L1} is independently deuterium, or halogen; or two R^{L1} on the same carbon are taken together to form an oxo.

In some embodiments of a compound of Formula (I), or a pharmaceutically acceptable salt, solvate, or stereoisomer thereof, p is 1. In some embodiments of a compound of Formula (I), or a pharmaceutically acceptable salt, solvate, or stereoisomer thereof, p is 2.

In some embodiments of a compound of Formula (I), or a pharmaceutically acceptable salt, solvate, or stereoisomer thereof, m is 0. In some embodiments of a compound of Formula (I), or a pharmaceutically acceptable salt, solvate, or stereoisomer thereof, m is 1 or 2. In some embodiments of a compound of Formula (I), or a pharmaceutically acceptable salt, solvate, or stereoisomer thereof, m is 1. In some embodiments of a compound of Formula (I), or a pharmaceutically acceptable salt, solvate, or stereoisomer thereof, m is 2.

In some embodiments of a compound of Formula (I), or a pharmaceutically acceptable salt, solvate, or stereoisomer thereof, each R⁵ is independently deuterium, halogen, —CN, —NO₂, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl; or two R⁵ on the same carbon are taken together to form an oxo. In some embodiments of a compound of Formula (I), or a pharmaceutically acceptable salt, solvate, or stereoisomer thereof, each R⁵ is independently deuterium, halogen, C₁-C₆alkyl, C₁-C₆haloalkyl, C₁-C₆deuteroalkyl, C₁-C₆hydroxyalkyl, or C₁-C₆aminoalkyl; or two R⁵ on the same carbon are taken together to form an oxo. In some embodiments of a compound of Formula (I), or a pharmaceutically acceptable salt, solvate, or stereoisomer thereof, each R⁵ is independently deuterium, halogen, or C₁-C₆alkyl; or two R⁵ on the same carbon are taken together to form an oxo.

In some embodiments of a compound of Formula (I), or a pharmaceutically acceptable salt, solvate, or stereoisomer thereof, Ring A is a 6- to 12-membered bicyclic ring optionally comprising 1-4 heteroatoms selected from the group consisting of O, S, and N. In some embodiments of a compound of Formula (I), or a pharmaceutically acceptable salt, solvate, or stereoisomer thereof, Ring A is a 6- to 12-membered bicyclic ring optionally comprising 1-4 heteroatoms selected from the group consisting of 0 and N. In some embodiments of a compound of Formula (I), or a pharmaceutically acceptable salt, solvate, or stereoisomer thereof, Ring A is a 6- to 12-membered bicyclic ring optionally comprising 1-4 heteroatoms selected from the group consisting of 0 and N. In some embodiments of a compound of Formula (I), or a pharmaceutically acceptable salt, solvate, or stereoisomer thereof, Ring A is a 6- to 12-membered bicyclic ring comprising 1 or 2 heteroatoms selected from the group consisting of 0 and N. In some embodiments of a compound of Formula (I), or a pharmaceutically acceptable salt, solvate, or stereoisomer thereof, Ring A is a 6- to 10-membered bicyclic ring comprising 1 heteroatom that is 0. In some embodiments of a compound of Formula (I), or a pharmaceutically acceptable salt, solvate, or stereoisomer thereof, Ring A is a 6- to 10-membered bicyclic ring comprising 1 heteroatom that is N.

In some embodiments of a compound of Formula (I), or a pharmaceutically acceptable salt, solvate, or stereoisomer thereof, Ring A is

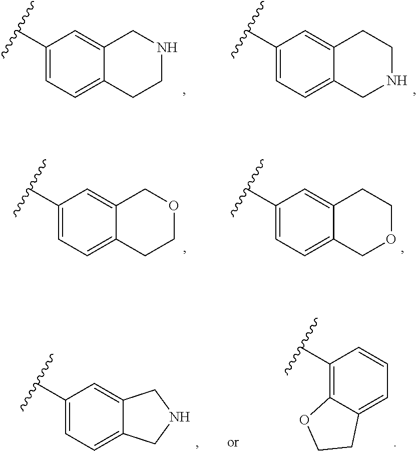 [SMILES: *c1ccc2c(c1)CNCC2 ; *c1ccc2c(c1)CCNC2 ; *c1ccc2c(c1)COCC2 ; *c1ccc2c(c1)CCOC2 ; *c1ccc2c(c1)CNC2 ; *c1cccc2c1OCC2]

In some embodiments of a compound of Formula (I), or a pharmaceutically acceptable salt, solvate, or stereoisomer thereof, Ring A is

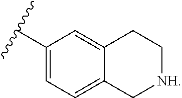 [SMILES: *c1ccc2c(c1)CCNC2]

In some embodiments of a compound of Formula (I), or a pharmaceutically acceptable salt, solvate, or stereoisomer thereof,

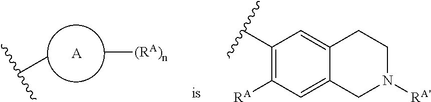

and R^{A′} is hydrogen or C₁-C₆alkyl.

In some embodiments of a compound of Formula (I), or a pharmaceutically acceptable salt, solvate, or stereoisomer thereof, each R^{A} is independently deuterium, halogen, —CN, —NO₂, —OH, —OR^{a}, —O(C₂-C₆alkylene)OR^{a}, —O(C₂-C₆alkylene)NR^{c}R^{d}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, cycloalkyl, heterocycloalkyl, aryl, and heteroaryl are independently substituted with one, two, or three R^{Aa}; or two R^{A} on the same carbon are taken together to form an oxo.

In some embodiments of a compound of Formula (I), or a pharmaceutically acceptable salt, solvate, or stereoisomer thereof, each R^{A} is independently deuterium, halogen, —CN, —NO₂, —OH, —OR^{a}, —O(C₂-C₆alkylene)OR^{a}, —O(C₂-C₆alkylene)NR^{c}R^{d}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆deuteroalkyl, C₁-C₆hydroxyalkyl, C₁-C₆aminoalkyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); or two R^{A} on the same carbon are taken together to form an oxo.

In some embodiments of a compound of Formula (I), or a pharmaceutically acceptable salt, solvate, or stereoisomer thereof, each R^{A} is independently halogen, —OH, —OR^{a}, —O(C₂-C₆alkylene)NR^{c}R^{d}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, cycloalkyl, or heterocycloalkyl; wherein each alkyl, cycloalkyl, and heterocycloalkyl are independently substituted with one, two, or three R^{Aa}; or two R^{A} on the same carbon are taken together to form an oxo.

In some embodiments of a compound of Formula (I), or a pharmaceutically acceptable salt, solvate, or stereoisomer thereof, each R^{A} is independently halogen, —OH, —OR^{a}, —O(C₂-C₆alkylene)NR^{c}R^{d}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆hydroxyalkyl, cycloalkyl, or heterocycloalkyl; or two R^{A} on the same carbon are taken together to form an oxo.

In some embodiments of a compound of Formula (I), or a pharmaceutically acceptable salt, solvate, or stereoisomer thereof, each R^{A} is independently halogen, —OH, —OR^{a}, —NR^{c}R^{d}, —C(═O)R^{a}, C₁-C₆alkyl, or C₁-C₆haloalkyl; wherein each alkyl are independently substituted with one, two, or three R^{Aa}; or two R^{A} on the same carbon are taken together to form an oxo.

In some embodiments of a compound of Formula (I), or a pharmaceutically acceptable salt, solvate, or stereoisomer thereof, each R^{A} is independently halogen, —OH, —OR^{a}, —NR^{c}R^{d}, —C(═O)R^{a}, or C₁-C₆alkyl; or two R^{A} on the same carbon are taken together to form an oxo.

In some embodiments of a compound of Formula (I), or a pharmaceutically acceptable salt, solvate, or stereoisomer thereof, each R^{A} is independently halogen, —OH, —OR^{a}, —NR^{c}R^{d}, C₁-C₆alkyl, or C₁-C₆haloalkyl; wherein each alkyl are independently substituted with one, two, or three R^{Aa}.

In some embodiments of a compound of Formula (I), or a pharmaceutically acceptable salt, solvate, or stereoisomer thereof, each R^{A} is independently halogen or C₁-C₆alkyl.

In some embodiments of a compound of Formula (I), or a pharmaceutically acceptable salt, solvate, or stereoisomer thereof, each R^{A} is independently halogen.

In some embodiments of a compound of Formula (I), or a pharmaceutically acceptable salt, solvate, or stereoisomer thereof, each R^{Aa} is independently deuterium, halogen, —CN, —OH, —OR^{a}, —NR^{c}R^{d} C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl; or two R^{Aa} on the same carbon are taken together to form an oxo.

In some embodiments of a compound of Formula (I), or a pharmaceutically acceptable salt, solvate, or stereoisomer thereof, each R^{Aa} is independently deuterium, halogen, —CN, —OH, —OR^{a}, —NR^{c}R^{d} C₁-C₆alkyl, C₁-C₆haloalkyl, or C₁-C₆deuteroalkyl; or two R^{Aa} on the same carbon are taken together to form an oxo.

In some embodiments of a compound of Formula (I), or a pharmaceutically acceptable salt, solvate, or stereoisomer thereof, each R^{Aa} is independently halogen, —OH, —OR^{a}, —NR^{c}R^{d}, or C₁-C₆alkyl.

In some embodiments of a compound of Formula (I), or a pharmaceutically acceptable salt, solvate, or stereoisomer thereof, n is 1-3. In some embodiments of a compound of Formula (I), or a pharmaceutically acceptable salt, solvate, or stereoisomer thereof, n is 1. In some embodiments of a compound of Formula (I), or a pharmaceutically acceptable salt, solvate, or stereoisomer thereof, n is 1 or 2. In some embodiments of a compound of Formula (I), or a pharmaceutically acceptable salt, solvate, or stereoisomer thereof, n is 2.

In some embodiments of a compound of Formula (I), or a pharmaceutically acceptable salt, solvate, or stereoisomer thereof,

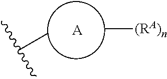

is selected from:

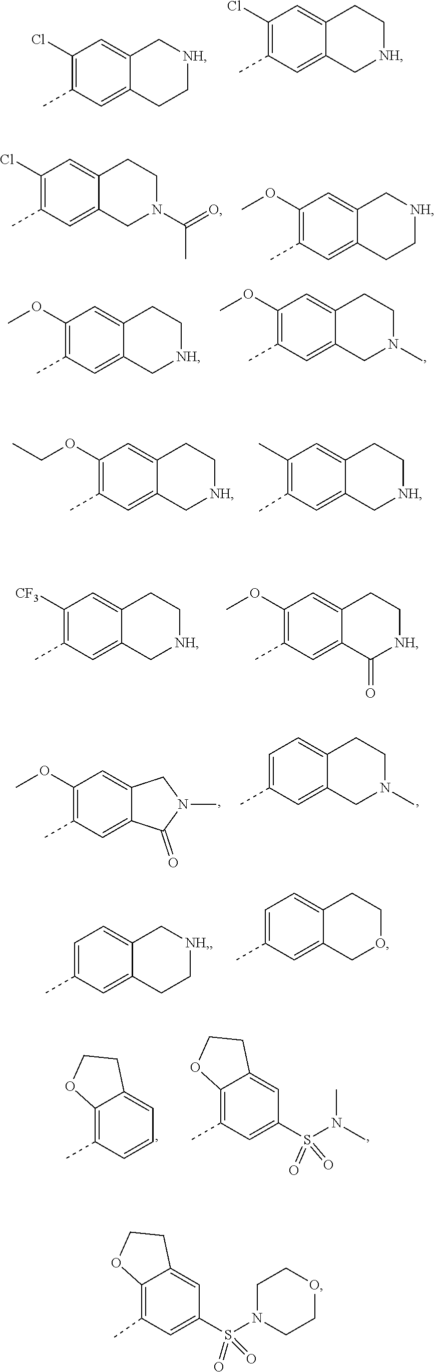

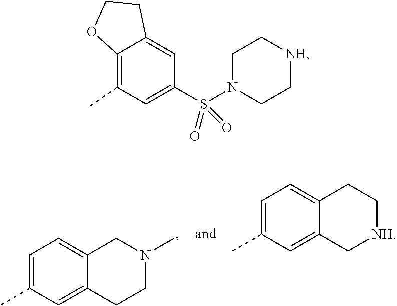

In some embodiments of a compound of Formula (I), or a pharmaceutically acceptable salt, solvate, or stereoisomer thereof,

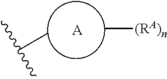

is selected from

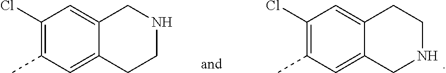

In some embodiments of a compound of Formula (I), or a pharmaceutically acceptable salt, solvate, or stereoisomer thereof,

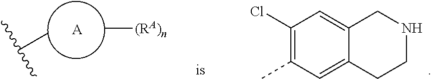

In some embodiments of a compound of Formula (I), or a pharmaceutically acceptable salt, solvate, or stereoisomer thereof,

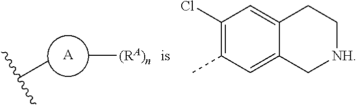

Also disclosed herein are compounds of Formula (IIa) or (IIb), or a pharmaceutically acceptable salt, solvate, or stereoisomer thereof:

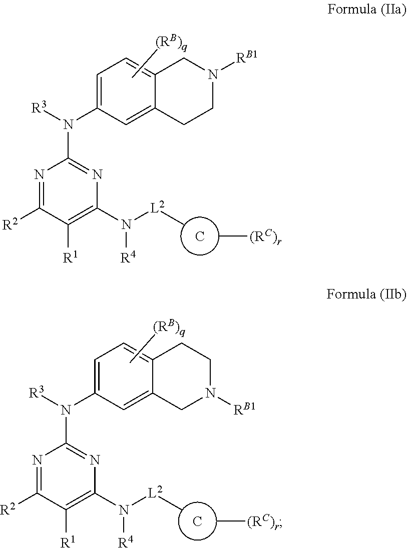

wherein:
R¹ is C₁-C₆haloalkyl or cycloalkyl;
R² is hydrogen, halogen, deuterium, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, or heterocycloalkyl;
R³ is hydrogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl;
R⁴ is hydrogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl;
each R^{B} is independently deuterium, halogen, —CN, —NO₂, —OH, —OR^{a}, —O(C₂-C₆alkylene)OR^{a}, —O(C₂-C₆alkylene)NR^{c}R^{d}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —NR^{b}C(═O)OR^{b}, —NR^{b}C(═O)NR^{c}R^{d}, —NHS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl are independently substituted with one, two, or three R^{Ba};
or two R^{B} on the same carbon are taken together to form an oxo;
each R^{Ba} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —NR^{b}C(═O)OR^{b}, —NR^{b}C(═O)NR^{c}R^{d}, —NHS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{Ba} on the same carbon are taken together to form an oxo;
R^{B1} is hydrogen, —S(═O)R^{a}, —S(═O)₂R^{a}, —S(═O)₂NR^{c}R^{d}, —C(═O)R^{a}, —C(═O)OR^{b}, —C(═O)NR^{C}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl);
Ring C is cycloalkyl, heterocycloalkyl, aryl, or heteroaryl;
each R^{c} is independently deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{c} on the same carbon are taken together to form an oxo;
L² is a C₁-C₄ alkylene optionally substituted with one, two, or three R^{L2};
each R^{L2} is independently deuterium, halogen, —CN, —NO₂, —OH, —OR^{a}, or —NR^{c}R^{d};
or two R^{L2} on the same carbon are taken together to form an oxo;
q is 0-4;
r is 0-4;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl;
or R^{c} and R^{d} are taken together with the atom to which they are attached to form a heterocycloalkyl optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl.

In some embodiments, the compound is of Formula (IIa), or a pharmaceutically acceptable salt, solvate, or stereoisomer thereof:

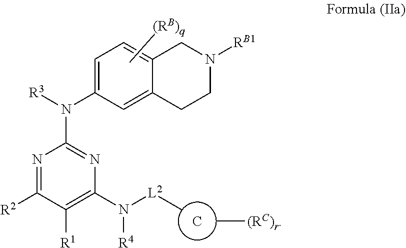

In some embodiments, the compound is of Formula (IIb), or a pharmaceutically acceptable salt, solvate, or stereoisomer thereof:

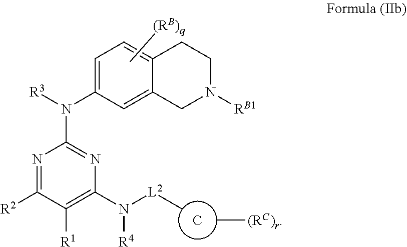

In some embodiments of a compound of Formula (IIa) or (IIb), or a pharmaceutically acceptable salt, solvate, or stereoisomer thereof, R² is hydrogen, halogen, deuterium, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl. In some embodiments of a compound of Formula (IIa) or (IIb), or a pharmaceutically acceptable salt, solvate, or stereoisomer thereof, R² is hydrogen, halogen, deuterium, C₁-C₆alkyl, C₁-C₆haloalkyl, or C₁-C₆deuteroalkyl. In some embodiments of a compound of Formula (IIa) or (IIb), or a pharmaceutically acceptable salt, solvate, or stereoisomer thereof, R² is hydrogen, halogen, or C₁-C₆alkyl. In some embodiments of a compound of Formula (IIa) or (IIb), or a pharmaceutically acceptable salt, solvate, or stereoisomer thereof, R² is hydrogen.

In some embodiments of a compound of Formula (IIa) or (IIb), or a pharmaceutically acceptable salt, solvate, or stereoisomer thereof, R³ is hydrogen, C₁-C₆alkyl, or C₁-C₆haloalkyl. In some embodiments of a compound of Formula (IIa) or (IIb), or a pharmaceutically acceptable salt, solvate, or stereoisomer thereof, R³ is hydrogen or C₁-C₆alkyl. In some embodiments of a compound of Formula (IIa) or (IIb), or a pharmaceutically acceptable salt, solvate, or stereoisomer thereof, R³ is C₁-C₆alkyl. In some embodiments of a compound of Formula (IIa) or (IIb), or a pharmaceutically acceptable salt, solvate, or stereoisomer thereof, R³ is hydrogen.

In some embodiments of a compound of Formula (IIa) or (IIb), or a pharmaceutically acceptable salt, solvate, or stereoisomer thereof, R⁴ is hydrogen, C₁-C₆alkyl, or C₁-C₆haloalkyl. In some embodiments of a compound of Formula (IIa) or (IIb), or a pharmaceutically acceptable salt, solvate, or stereoisomer thereof, R⁴ is hydrogen or C₁-C₆alkyl. In some embodiments of a compound of Formula (IIa) or (IIb), or a pharmaceutically acceptable salt, solvate, or stereoisomer thereof, R⁴ is C₁-C₆alkyl. In some embodiments of a compound of Formula (IIa) or (IIb), or a pharmaceutically acceptable salt, solvate, or stereoisomer thereof, R⁴ is hydrogen.

In some embodiments of a compound of Formula (IIa) or (IIb), or a pharmaceutically acceptable salt, solvate, or stereoisomer thereof, R¹ is C₁-C₆haloalkyl. In some embodiments of a compound of Formula (IIa) or (IIb), or a pharmaceutically acceptable salt, solvate, or stereoisomer thereof, R¹ is CF₃. In some embodiments of a compound of Formula (IIa) or (IIb), or a pharmaceutically acceptable salt, solvate, or stereoisomer thereof, R¹ is cycloalkyl. In some embodiments of a compound of Formula (IIa) or (IIb), or a pharmaceutically acceptable salt, solvate, or stereoisomer thereof, R¹ is cyclopropyl.

In some embodiments of a compound of Formula (IIa) or (IIb), or a pharmaceutically acceptable salt, solvate, or stereoisomer thereof, L² is a C₃ alkylene optionally substituted with one, two, or three R^{L2}. In some embodiments of a compound of Formula (IIa) or (IIb), or a pharmaceutically acceptable salt, solvate, or stereoisomer thereof, L² is a C₄ alkylene optionally substituted with one, two, or three R^{L2}.

In some embodiments of a compound of Formula (IIa) or (IIb), or a pharmaceutically acceptable salt, solvate, or stereoisomer thereof, L² is C₃-C₄ alkylene. In some embodiments of a compound of Formula (IIa) or (IIb), or a pharmaceutically acceptable salt, solvate, or stereoisomer thereof, L² is C₃ alkylene. In some embodiments of a compound of Formula (IIa) or (IIb), or a pharmaceutically acceptable salt, solvate, or stereoisomer thereof, L² is C₄ alkylene.

In some embodiments of a compound of Formula (IIa) or (IIb), or a pharmaceutically acceptable salt, solvate, or stereoisomer thereof, each R^{L2} is independently deuterium, halogen, —OH, —OR^{a}, or —NR^{c}R^{d}; or two R^{L2} on the same carbon are taken together to form an oxo. In some embodiments of a compound of Formula (IIa) or (IIb), or a pharmaceutically acceptable salt, solvate, or stereoisomer thereof, each R^{L2} is independently deuterium, or halogen; or two R^{L2} on the same carbon are taken together to form an oxo. In some embodiments of a compound of Formula (IIa) or (IIb), or a pharmaceutically acceptable salt, solvate, or stereoisomer thereof, two R^{L2} on the same carbon are taken together to form an oxo.

In some embodiments of a compound of Formula (IIa) or (IIb), or a pharmaceutically acceptable salt, solvate, or stereoisomer thereof,

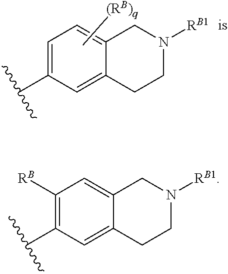

In some embodiments of a compound of Formula (IIa) or (IIb), or a pharmaceutically acceptable salt, solvate, or stereoisomer thereof,

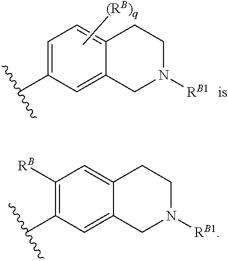

In some embodiments of a compound of Formula (IIa) or (IIb), or a pharmaceutically acceptable salt, solvate, or stereoisomer thereof, q is 0-2. In some embodiments of a compound of Formula (IIa) or (IIb), or a pharmaceutically acceptable salt, solvate, or stereoisomer thereof, q is 0 or 1. In some embodiments of a compound of Formula (IIa) or (IIb), or a pharmaceutically acceptable salt, solvate, or stereoisomer thereof, q is 1 or 2. In some embodiments of a compound of Formula (IIa) or (IIb), or a pharmaceutically acceptable salt, solvate, or stereoisomer thereof, q is 1.

In some embodiments of a compound of Formula (IIa) or (IIb), or a pharmaceutically acceptable salt, solvate, or stereoisomer thereof, R^{B1} is hydrogen, —C(═O)R^{a}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl). In some embodiments of a compound of Formula (IIa) or (IIb), or a pharmaceutically acceptable salt, solvate, or stereoisomer thereof, R^{B1} is hydrogen, —C(═O)R^{a}, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl. In some embodiments of a compound of Formula (IIa) or (IIb), or a pharmaceutically acceptable salt, solvate, or stereoisomer thereof, R^{B1} is hydrogen, —C(═O)R^{a}, C₁-C₆alkyl, or C₁-C₆haloalkyl. In some embodiments of a compound of Formula (IIa) or (IIb), or a pharmaceutically acceptable salt, solvate, or stereoisomer thereof, R^{B1} is hydrogen or C₁-C₆alkyl. In some embodiments of a compound of Formula (IIa) or (IIb), or a pharmaceutically acceptable salt, solvate, or stereoisomer thereof, R^{B1} is hydrogen.

In some embodiments of a compound of Formula (IIa) or (IIb), or a pharmaceutically acceptable salt, solvate, or stereoisomer thereof, each R^{B} is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, cycloalkyl, heterocycloalkyl, aryl, and heteroaryl are independently substituted with one, two, or three R^{Ba}. In some embodiments of a compound of Formula (IIa) or (IIb), or a pharmaceutically acceptable salt, solvate, or stereoisomer thereof, each R^{B} is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl are independently substituted with one, two, or three R^{Ba}. In some embodiments of a compound of Formula (IIa) or (IIb), or a pharmaceutically acceptable salt, solvate, or stereoisomer thereof, each R^{B} is independently halogen, —OH, —OR^{a}, —NR^{c}R^{d}, —C(═O)R^{a}, C₁-C₆alkyl, or C₁-C₆haloalkyl; wherein each alkyl are independently substituted with one, two, or three R^{Ba}; or two R^{B} on the same carbon are taken together to form an oxo. In some embodiments of a compound of Formula (IIa) or (IIb), or a pharmaceutically acceptable salt, solvate, or stereoisomer thereof, each R^{B} is independently halogen, —OH, —OR^{a}, —NR^{c}R^{d}, C₁-C₆alkyl, or C₁-C₆haloalkyl; wherein each alkyl are independently substituted with one, two, or three R^{Ba}. In some embodiments of a compound of Formula (IIa) or (IIb), or a pharmaceutically acceptable salt, solvate, or stereoisomer thereof, each R^{B} is independently halogen or C₁-C₆alkyl. In some embodiments of a compound of Formula (IIa) or (IIb), or a pharmaceutically acceptable salt, solvate, or stereoisomer thereof, each R^{B} is independently halogen.

In some embodiments of a compound of Formula (IIa) or (IIb), or a pharmaceutically acceptable salt, solvate, or stereoisomer thereof, Ring C is cycloalkyl or heterocycloalkyl. In some embodiments of a compound of Formula (IIa) or (IIb), or a pharmaceutically acceptable salt, solvate, or stereoisomer thereof, Ring C is aryl or heteroaryl. In some embodiments of a compound of Formula (IIa) or (IIb), or a pharmaceutically acceptable salt, solvate, or stereoisomer thereof, Ring C is heteroaryl. In some embodiments of a compound of Formula (IIa) or (IIb), or a pharmaceutically acceptable salt, solvate, or stereoisomer thereof, Ring C is heterocycloalkyl.

In some embodiments of a compound of Formula (IIa) or (IIb), or a pharmaceutically acceptable salt, solvate, or stereoisomer thereof, each R^{c} is independently deuterium, halogen, —CN, —OH, —OR^{a}, —NR^{C}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl; or two R^{c} on the same carbon are taken together to form an oxo. In some embodiments of a compound of Formula (IIa) or (IIb), or a pharmaceutically acceptable salt, solvate, or stereoisomer thereof, each R^{c} is independently deuterium, halogen, —CN, —OH, —OR^{a}, —NR^{c}R^{d}, C₁-C₆alkyl, or C₁-C₆haloalkyl; or two R^{c} on the same carbon are taken together to form an oxo. In some embodiments of a compound of Formula (IIa) or (IIb), or a pharmaceutically acceptable salt, solvate, or stereoisomer thereof, two R^{c} on the same carbon are taken together to form an oxo.

In some embodiments of a compound of Formula (I), (IIa), or (IIb), or a pharmaceutically acceptable salt, solvate, or stereoisomer thereof, each R^{a} is independently C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl. In some embodiments of a compound of Formula (I), (IIa), or (IIb), or a pharmaceutically acceptable salt, solvate, or stereoisomer thereof, each R^{a} is independently C₁-C₆alkyl, C₁-C₆haloalkyl, C₁-C₆deuteroalkyl, C₁-C₆hydroxyalkyl, C₁-C₆aminoalkyl; wherein each alkyl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, or —C(═O)OCH₃. In some embodiments of a compound of Formula (I), (IIa), or (IIb), or a pharmaceutically acceptable salt, solvate, or stereoisomer thereof, each R^{a} is independently C₁-C₆alkyl or C₁-C₆haloalkyl; wherein each alkyl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, or —C(═O)OCH₃. In some embodiments of a compound of Formula (I), (IIa), or (IIb), or a pharmaceutically acceptable salt, solvate, or stereoisomer thereof, each R^{a} is independently C₁-C₆alkyl or C₁-C₆haloalkyl. In some embodiments of a compound of Formula (I), (IIa), or (IIb), or a pharmaceutically acceptable salt, solvate, or stereoisomer thereof, each R^{a} is independently C₁-C₆alkyl.

In some embodiments of a compound of Formula (I), (IIa), or (IIb), or a pharmaceutically acceptable salt, solvate, or stereoisomer thereof, each R^{b} is independently hydrogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl. In some embodiments of a compound of Formula (I), (IIa), or (IIb), or a pharmaceutically acceptable salt, solvate, or stereoisomer thereof, each R^{b} is independently hydrogen, C₁-C₆alkyl, C₁-C₆haloalkyl, C₁-C₆deuteroalkyl, C₁-C₆hydroxyalkyl, C₁-C₆aminoalkyl; wherein each alkyl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, or —C(═O)OCH₃. In some embodiments of a compound of Formula (I), (IIa), or (IIb), or a pharmaceutically acceptable salt, solvate, or stereoisomer thereof, each R^{b} is independently hydrogen, C₁-C₆alkyl or C₁-C₆haloalkyl; wherein each alkyl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, or —C(═O)OCH₃. In some embodiments of a compound of Formula (I), (IIa), or (IIb), or a pharmaceutically acceptable salt, solvate, or stereoisomer thereof, each R^{b} is independently hydrogen, C₁-C₆alkyl, or C₁-C₆haloalkyl. In some embodiments of a compound of Formula (I), (IIa), or (IIb), or a pharmaceutically acceptable salt, solvate, or stereoisomer thereof, each R^{b} is independently hydrogen or C₁-C₆alkyl. In some embodiments of a compound of Formula (I), (IIa), or (IIb), or a pharmaceutically acceptable salt, solvate, or stereoisomer thereof, each R^{b} is hydrogen. In some embodiments of a compound of Formula (I), (IIa), or (IIb), or a pharmaceutically acceptable salt, solvate, or stereoisomer thereof, each R^{b} is independently C₁-C₆alkyl.

In some embodiments of a compound of Formula (I), (IIa), or (IIb), or a pharmaceutically acceptable salt, solvate, or stereoisomer thereof, each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl. In some embodiments of a compound of Formula (I), (IIa), or (IIb), or a pharmaceutically acceptable salt, solvate, or stereoisomer thereof, each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl; wherein each alkyl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, or —C(═O)OCH₃. In some embodiments of a compound of Formula (I), (IIa), or (IIb), or a pharmaceutically acceptable salt, solvate, or stereoisomer thereof, each R^{c} and R^{d} are independently hydrogen, C₁-C₆alkyl or C₁-C₆haloalkyl; wherein each alkyl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, or —C(═O)OCH₃. In some embodiments of a compound of Formula (I), (IIa), or (IIb), or a pharmaceutically acceptable salt, solvate, or stereoisomer thereof, each R^{c} and R^{d} are independently hydrogen, C₁-C₆alkyl, or C₁-C₆haloalkyl. In some embodiments of a compound of Formula (I), (IIa), or (IIb), or a pharmaceutically acceptable salt, solvate, or stereoisomer thereof, each R^{c} and R^{d} are independently hydrogen or C₁-C₆alkyl. In some embodiments of a compound of Formula (I), (IIa), or (IIb), or a pharmaceutically acceptable salt, solvate, or stereoisomer thereof, each R^{c} and R^{d} are hydrogen. In some embodiments of a compound of Formula (I), (IIa), or (IIb), or a pharmaceutically acceptable salt, solvate, or stereoisomer thereof, each R^{c} and R^{d} are independently C₁-C₆alkyl.

In some embodiments of a compound of Formula (I), (IIa), or (IIb), or a pharmaceutically acceptable salt, solvate, or stereoisomer thereof, R^{c} and R^{d} are taken together with the atom to which they are attached to form a heterocycloalkyl optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl.

Disclosed herein is a compound selected from the group consisting of:

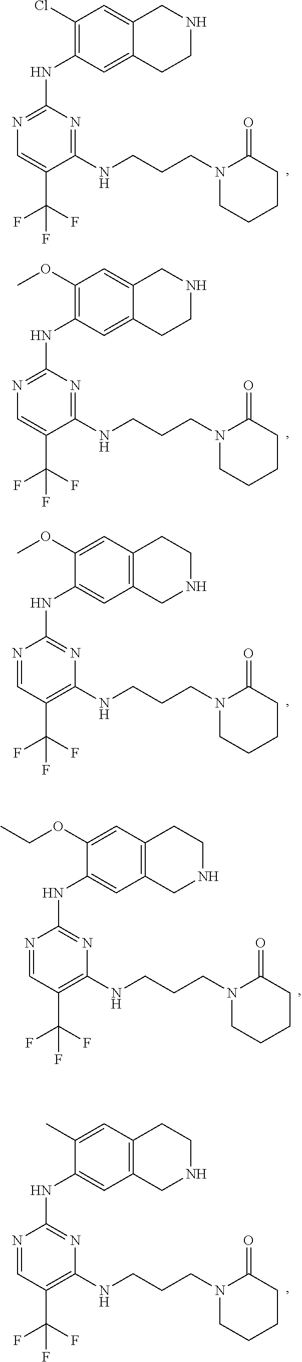

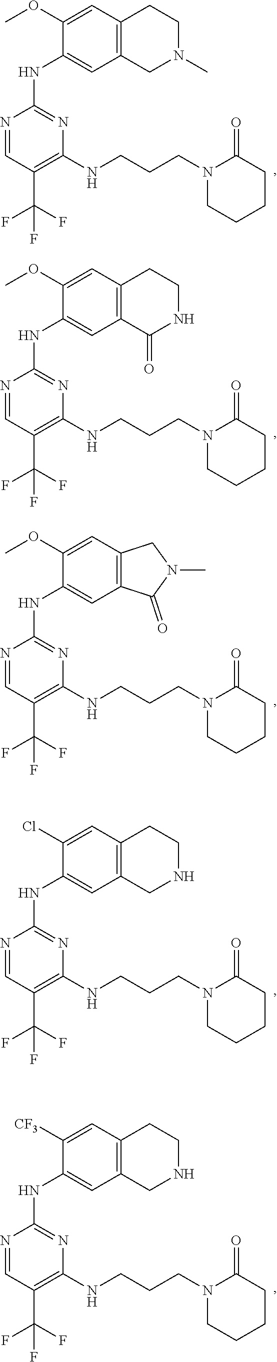

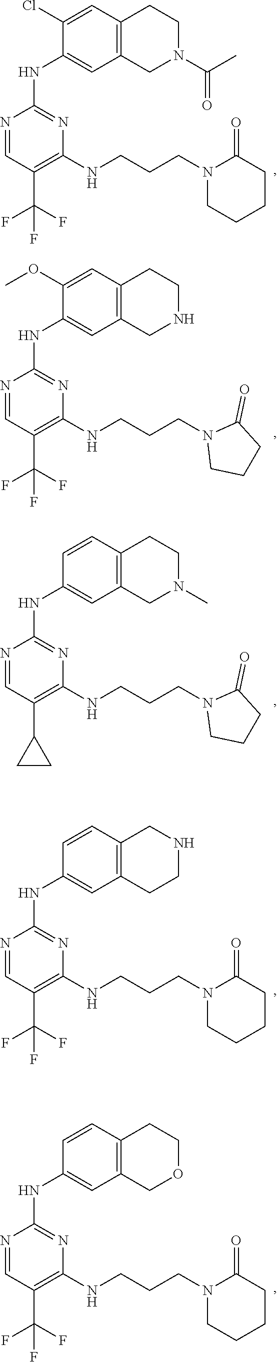

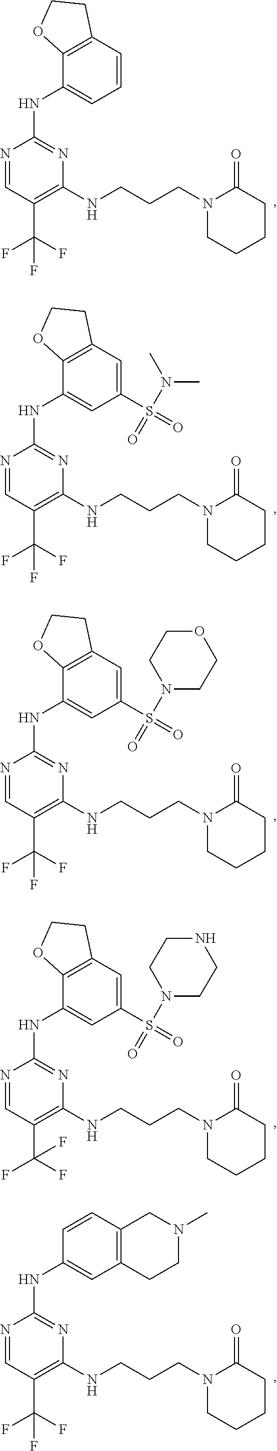

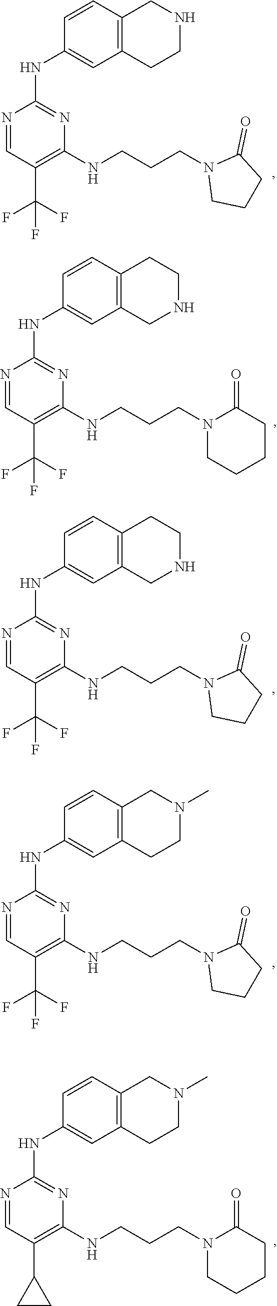 [SMILES: O=C1CCCN1CCCNc1nc(Nc2ccc3c(c2)CCNC3)ncc1C(F)(F)F ; O=C1CCCCN1CCCNc1nc(Nc2ccc3c(c2)CNCC3)ncc1C(F)(F)F ; O=C1CCCN1CCCNc1nc(Nc2ccc3c(c2)CNCC3)ncc1C(F)(F)F ; CN1CCc2cc(Nc3ncc(C(F)(F)F)c(NCCCN4CCCC4=O)n3)ccc2C1 ; CN1CCc2cc(Nc3ncc(C4CC4)c(NCCCN4CCCCC4=O)n3)ccc2C1]

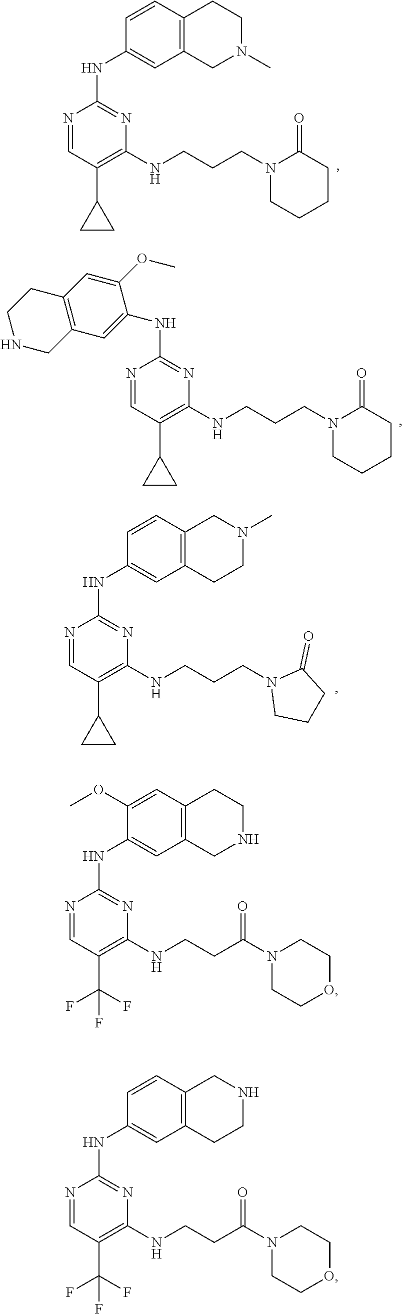

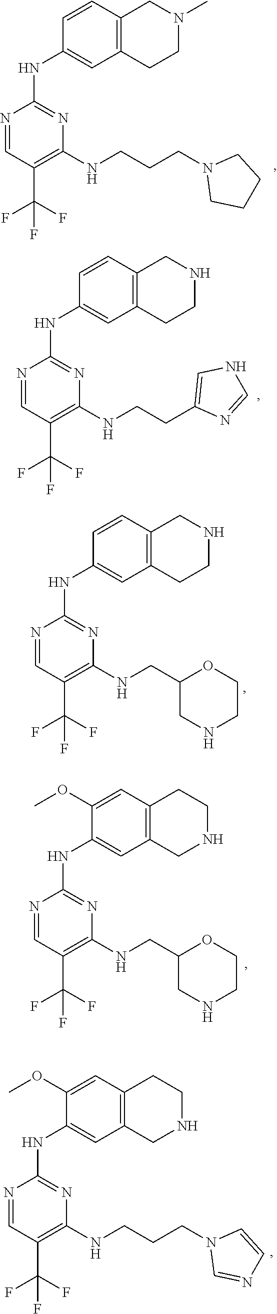

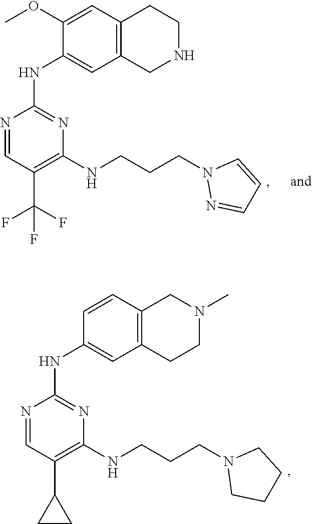 [SMILES: COc1cc2c(cc1Nc1ncc(C(F)(F)F)c(NCCCn3cccn3)n1)CNCC2 ; CN1CCc2cc(Nc3ncc(C4CC4)c(NCCCN4CCCC4)n3)ccc2C1]

or a pharmaceutically acceptable salt, solvate, or stereoisomer thereof.

Disclosed herein is compound selected from the group consisting of:

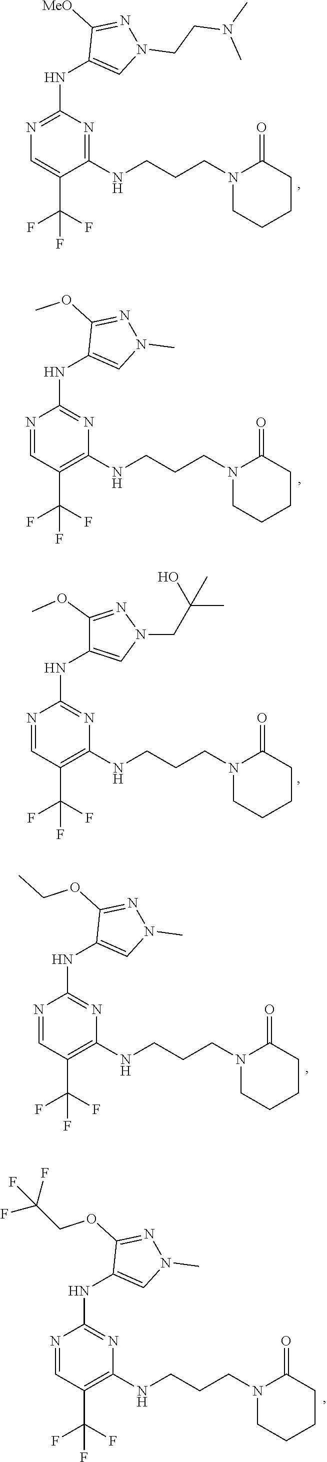 [SMILES: COc1nn(CCN(C)C)cc1Nc1ncc(C(F)(F)F)c(NCCCN2CCCCC2=O)n1 ; COc1nn(C)cc1Nc1ncc(C(F)(F)F)c(NCCCN2CCCCC2=O)n1 ; COc1nn(CC(C)(C)O)cc1Nc1ncc(C(F)(F)F)c(NCCCN2CCCCC2=O)n1 ; CCOc1nn(C)cc1Nc1ncc(C(F)(F)F)c(NCCCN2CCCCC2=O)n1 ; Cn1cc(Nc2ncc(C(F)(F)F)c(NCCCN3CCCCC3=O)n2)c(OCC(F)(F)F)n1]

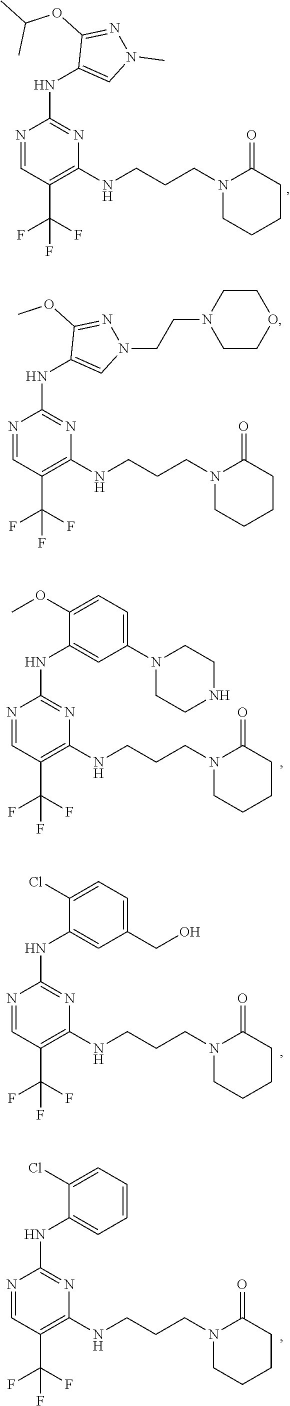 [SMILES: CC(C)Oc1nn(C)cc1Nc1ncc(C(F)(F)F)c(NCCCN2CCCCC2=O)n1 ; COc1nn(CCN2CCOCC2)cc1Nc1ncc(C(F)(F)F)c(NCCCN2CCCCC2=O)n1 ; COc1ccc(N2CCNCC2)cc1Nc1ncc(C(F)(F)F)c(NCCCN2CCCCC2=O)n1 ; O=C1CCCCN1CCCNc1nc(Nc2cc(CO)ccc2Cl)ncc1C(F)(F)F ; O=C1CCCCN1CCCNc1nc(Nc2ccccc2Cl)ncc1C(F)(F)F]

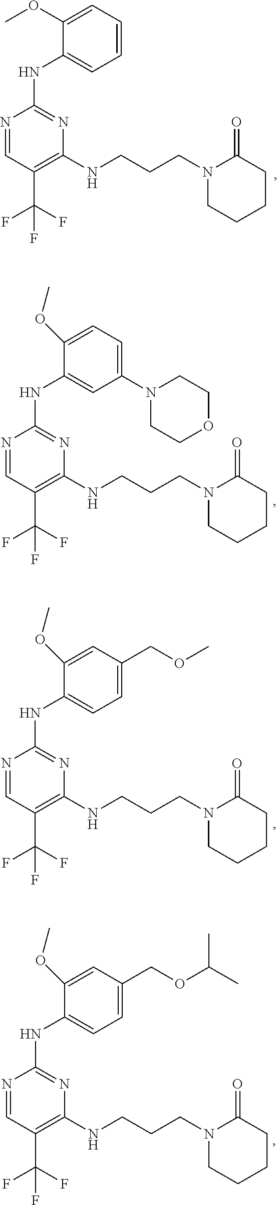 [SMILES: COc1ccccc1Nc1ncc(C(F)(F)F)c(NCCCN2CCCCC2=O)n1 ; COc1ccc(N2CCOCC2)cc1Nc1ncc(C(F)(F)F)c(NCCCN2CCCCC2=O)n1 ; COCc1ccc(Nc2ncc(C(F)(F)F)c(NCCCN3CCCCC3=O)n2)c(OC)c1 ; COc1cc(COC(C)C)ccc1Nc1ncc(C(F)(F)F)c(NCCCN2CCCCC2=O)n1]

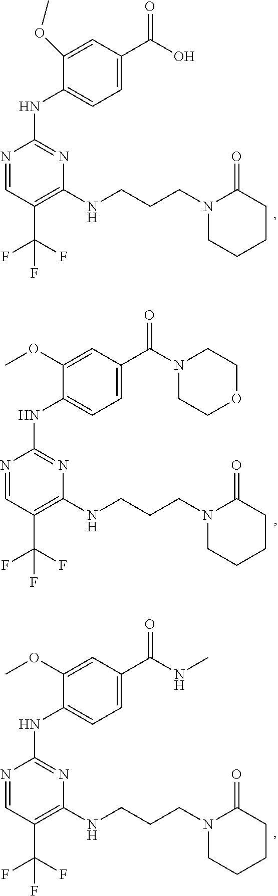 [SMILES: COc1cc(C(=O)O)ccc1Nc1ncc(C(F)(F)F)c(NCCCN2CCCCC2=O)n1 ; COc1cc(C(=O)N2CCOCC2)ccc1Nc1ncc(C(F)(F)F)c(NCCCN2CCCCC2=O)n1 ; CNC(=O)c1ccc(Nc2ncc(C(F)(F)F)c(NCCCN3CCCCC3=O)n2)c(OC)c1]

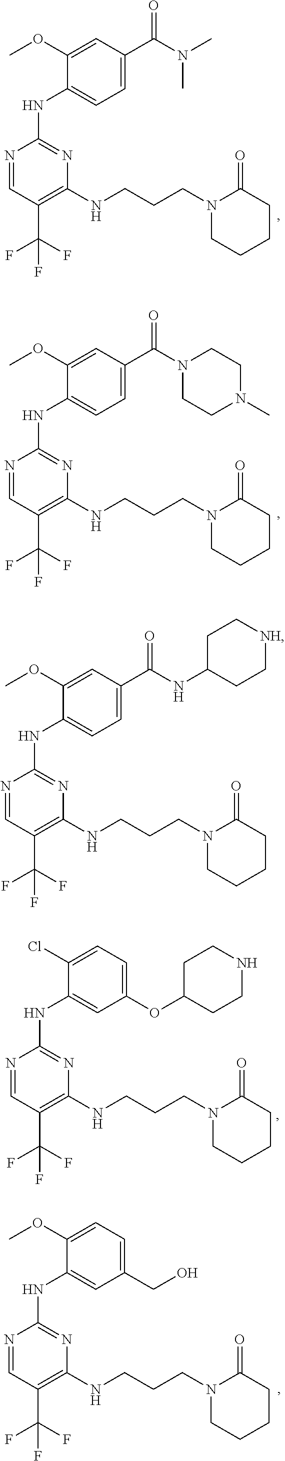

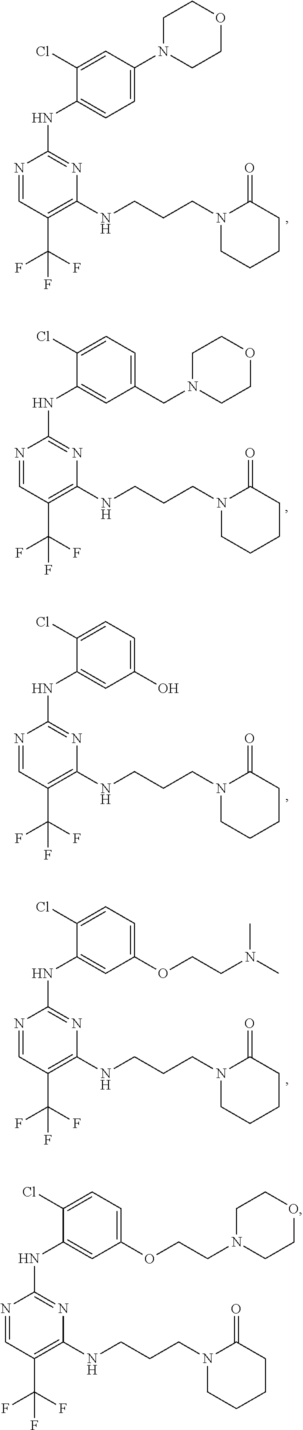 [SMILES: O=C1CCCCN1CCCNc1nc(Nc2ccc(N3CCOCC3)cc2Cl)ncc1C(F)(F)F ; O=C1CCCCN1CCCNc1nc(Nc2cc(CN3CCOCC3)ccc2Cl)ncc1C(F)(F)F ; O=C1CCCCN1CCCNc1nc(Nc2cc(O)ccc2Cl)ncc1C(F)(F)F ; CN(C)CCOc1ccc(Cl)c(Nc2ncc(C(F)(F)F)c(NCCCN3CCCCC3=O)n2)c1 ; O=C1CCCCN1CCCNc1nc(Nc2cc(OCCN3CCOCC3)ccc2Cl)ncc1C(F)(F)F]

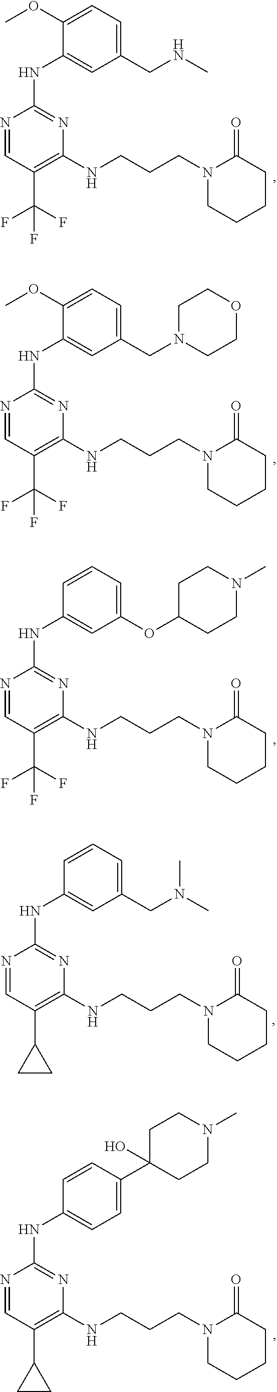 [SMILES: CNCc1ccc(OC)c(Nc2ncc(C(F)(F)F)c(NCCCN3CCCCC3=O)n2)c1 ; COc1ccc(CN2CCOCC2)cc1Nc1ncc(C(F)(F)F)c(NCCCN2CCCCC2=O)n1 ; CN1CCC(Oc2cccc(Nc3ncc(C(F)(F)F)c(NCCCN4CCCCC4=O)n3)c2)CC1 ; CN(C)Cc1cccc(Nc2ncc(C3CC3)c(NCCCN3CCCCC3=O)n2)c1 ; CN1CCC(O)(c2ccc(Nc3ncc(C4CC4)c(NCCCN4CCCCC4=O)n3)cc2)CC1]

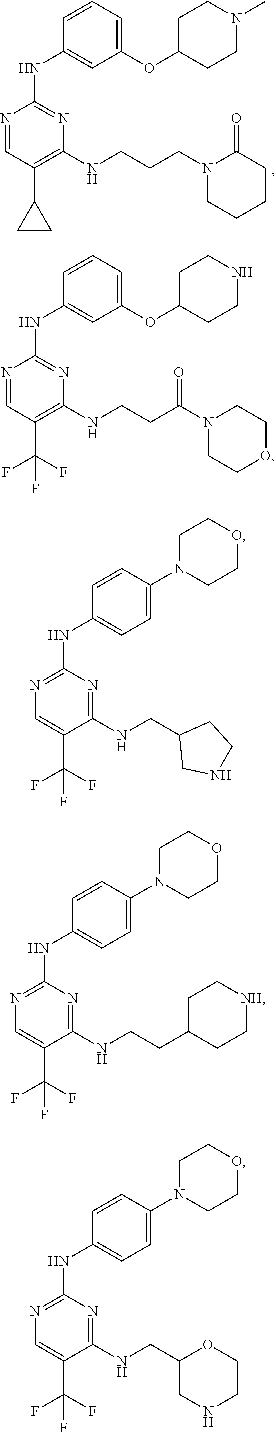

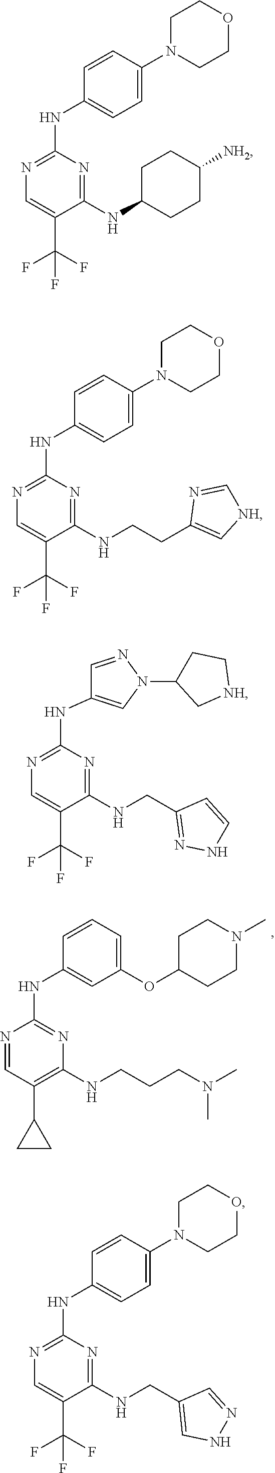

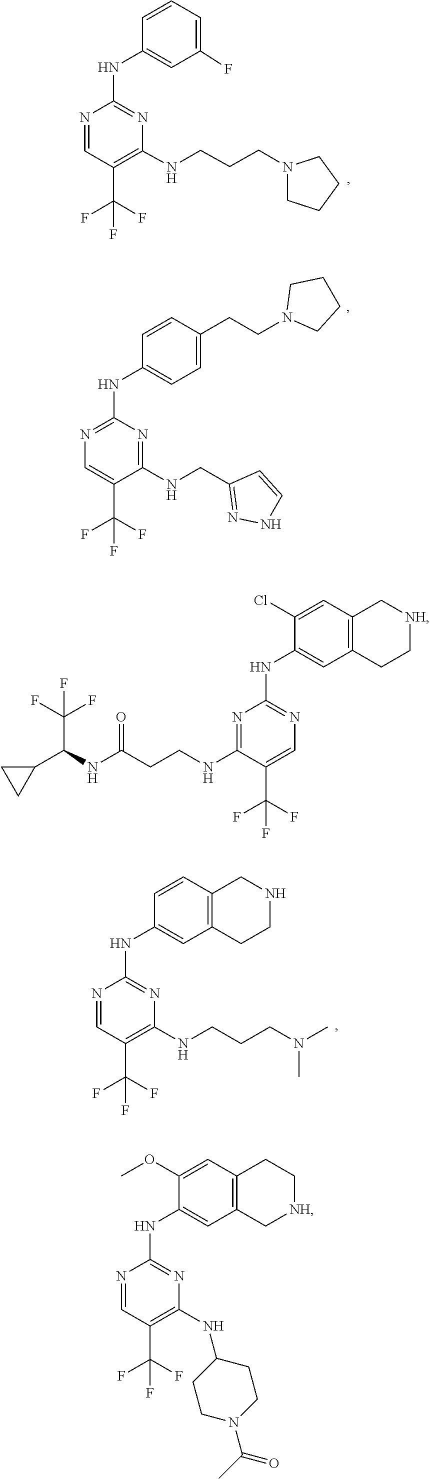 [SMILES: Fc1cccc(Nc2ncc(C(F)(F)F)c(NCCCN3CCCC3)n2)c1 ; FC(F)(F)c1cnc(Nc2ccc(CCN3CCCC3)cc2)nc1NCc1cc[nH]n1 ; O=C(CCNc1nc(Nc2cc3c(cc2Cl)CNCC3)ncc1C(F)(F)F)N[C@@H](C1CC1)C(F)(F)F ; CN(C)CCCNc1nc(Nc2ccc3c(c2)CCNC3)ncc1C(F)(F)F ; COc1cc2c(cc1Nc1ncc(C(F)(F)F)c(NC3CCN(C(C)=O)CC3)n1)CNCC2]

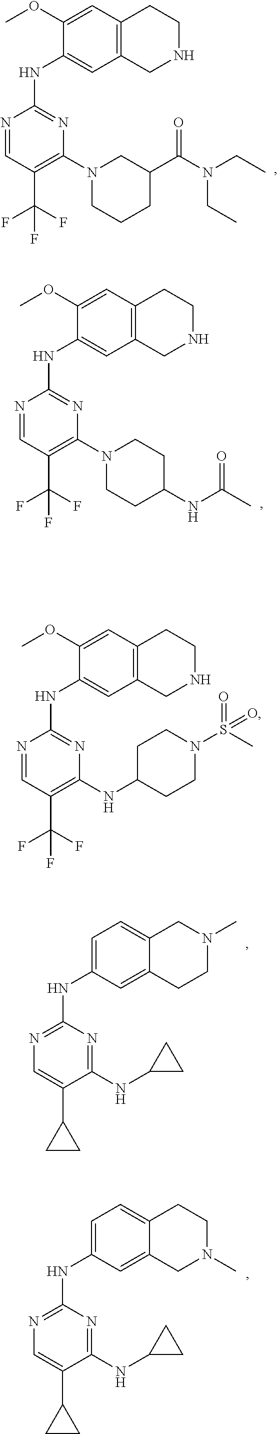 [SMILES: CCN(CC)C(=O)C1CCCN(c2nc(Nc3cc4c(cc3OC)CCNC4)ncc2C(F)(F)F)C1 ; COc1cc2c(cc1Nc1ncc(C(F)(F)F)c(N3CCC(NC(C)=O)CC3)n1)CNCC2 ; COc1cc2c(cc1Nc1ncc(C(F)(F)F)c(NC3CCN(S(C)(=O)=O)CC3)n1)CNCC2 ; CN1CCc2cc(Nc3ncc(C4CC4)c(NC4CC4)n3)ccc2C1 ; CN1CCc2ccc(Nc3ncc(C4CC4)c(NC4CC4)n3)cc2C1]

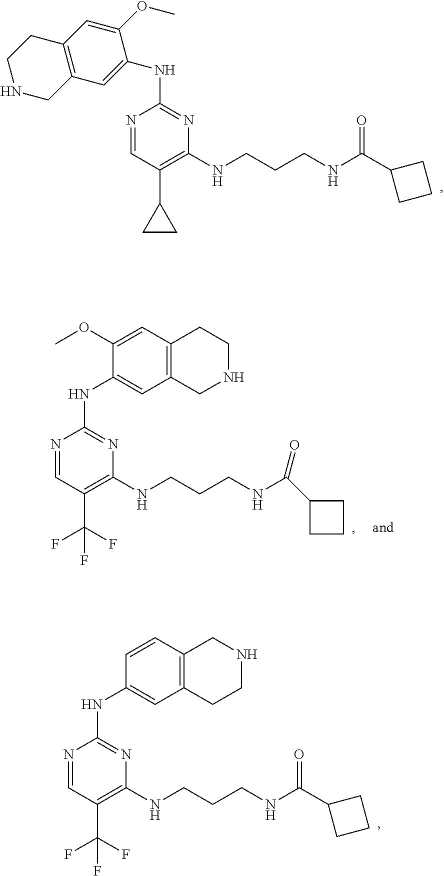 [SMILES: COc1cc2c(cc1Nc1ncc(C3CC3)c(NCCCNC(=O)C3CCC3)n1)CNCC2 ; COc1cc2c(cc1Nc1ncc(C(F)(F)F)c(NCCCNC(=O)C3CCC3)n1)CNCC2 ; O=C(NCCCNc1nc(Nc2ccc3c(c2)CCNC3)ncc1C(F)(F)F)C1CCC1]

or a pharmaceutically acceptable salt, solvate, or stereoisomer thereof.

### Further Forms of Compounds Disclosed Herein

### Isomers Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions.

Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., 2H, produces certain therapeutic advantages resulting from greater metabolic stability, for example increased in vivo half-life or reduced dosage requirements.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol.

In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist.

All tautomeric forms of the compounds disclosed herein are contemplated. The exact ratio of the tautomers depends on several factors, including temperature, solvent, and pH.

### Method of Treatment

Disclosed herein is a method of treating cancer sensitive to ULK1/2 inhibition in a subject in need thereof. Some embodiments of the disclosure include methods for treating abnormal cell growth in a subject comprising administering to the subject a therapeutically effective amount of a compound as described herein or a pharmaceutically acceptable salt, solvate, or stereoisomer thereof. In certain such embodiments, the abnormal cell growth is cancer, and in certain of those embodiments the cancer is lung cancer, pancreatic cancer, skin cancer, including melanoma, cancer of the head or neck, ovarian cancer, rectal cancer, colon cancer, breast cancer, cancer of the thyroid gland, chronic or acute leukaemia, and renal cell carcinoma. Such cancers may be KRAS associated cancers. In some embodiments, the cancer comprises a solid tumor. Of particular interest are cancers such as lung cancer, colon cancer, pancreatic cancer, and ovarian cancer. In some embodiment the method of treating cancer is a method of treating chronic myeloid leukaemia. In some embodiments, the cancer comprises a liquid tumor. In some embodiments, the cancer is chronic myeloid leukaemia.

In an embodiment the compound for use as described above is administered in combination with one or more additional anticancer agents. Such additional anticancer agents include compounds derived from the following classes: mitotic inhibitors, alkylating agents, antimetabolites, antitumor antibiotics, anti-angiogenesis agents, topoisomerase I and II inhibitors, plant alkaloids, hormonal agents and antagonists, growth factor inhibitors, radiation, signal transduction inhibitors, such as inhibitors of protein tyrosine kinases and/or serine/threonine kinases and/or phosphatases, cell cycle inhibitors, biological response modifiers, enzyme inhibitors, antisense oligonucleotides or oligonucleotide derivatives, cytotoxics, immuno-oncology agents, and the like. In some embodiments, the additional anti-cancer agent is a tyrosine kinase inhibitor. In a more preferred embodiment, the tyrosine kinase inhibitor is selected from imatinib and nilotinib. In embodiments the additional anti-cancer agent is administered concurrently, sequentially, or separately to the compound or pharmaceutically acceptable salt thereof. In an embodiment a further cancer treatment is radiotherapy.

In some embodiments, one or more compounds disclosed herein are administered to subjects having cancer that comprises one or more alterations in the MAPK pathway, including cancers having alternations in one or more of the RAS, SHP2, RAF, MEK, and ERK pathways. In some embodiments, the cancer in the subject has one or more alterations in the RAS pathway. In some embodiments, the cancer in the subject has one or more alterations in the RAF pathway. In some embodiments, the cancer in the subject has one or more alterations in the MEK pathway. In some embodiments, the cancer in the subject has one or more alterations in the ERK pathway. In some embodiments, one or more compounds disclosed herein are administered to subjects having cancer that is driven by cellular signalling in the MAPK pathway.

In some embodiments, one or more compounds disclosed herein are administered to subjects having cancer that comprises one or more alterations in the PI3K-AKT pathway, including cancers having alternations in one or more of the PI3K, PTEN, and AKT pathways. In some embodiments, the cancer in the subject has one or more alterations in the PI3K pathway. In some embodiments, the cancer in the subject has one or more alterations in the PTEN pathway. In some embodiments, the cancer in the subject has one or more alterations in the AKT pathway.

In some embodiments, one or more compounds disclosed herein are administered to subjects having cancer that comprises one or more alterations in the mTOR pathway.

In some embodiments, the cancer in the subject has one or more alterations in the RAS pathway, including mutations to KRAS, including G12C, G12D, and G12V mutations. KRAS inhibitors that may be used in combination with the compounds disclosed herein include, but are not limited to, one or more of AMG 510, MRTX849, and GDC-6036.

In some embodiments, the cancer in the subject has one or more alterations in the RAF pathway, including mutations to BRAF, including BRAF V600E. BRAF inhibitors that may be used in combination with the compounds disclosed herein include, but are not limited to, one or more of encorafenib, dabrafenib, and vemurafenib.

In some embodiments, the cancer in the subject has one or more alterations in the ERK pathway. ERK inhibitors that may be used in combination with the compounds disclosed herein include, but are not limited to, one or more of ulixertinib, ASN007, LY3214996, AZ13767370, MK-8353, and LTT462.

In some embodiments, the cancer in the subject has one or more alterations in the MEK pathway. MEK inhibitors that may be used in combination with the compounds disclosed herein include, but are not limited to, one or more of trametinib, binimetinib, cobimetinib, and selumetinib.

In some embodiments, the cancer in the subject may be treated by administration of a compound of the disclosure in combination with inhibitors of mammalian target of rapamycin (mTOR). Among mTOR inhibitors that may be used include, but are not limited, to sirolimus, everolimus, temsirolimus, and ridaforolimus (AP23573 and MK-8669).

The present disclosure also provides for method of treating a cancer sensitive to ULK1/2 inhibition by administering (i) a compound or pharmaceutically acceptable salt, solvate, or stereoisomer thereof as defined in any of the above embodiments and (ii) an additional anti-cancer agent, for simultaneous, separate or sequential use. In some embodiments, the cancer is lung cancer, pancreatic cancer, skin cancer, including melanoma, cancer of the head or neck, ovarian cancer, rectal cancer, colon cancer, breast cancer, cancer of the thyroid gland, chronic or acute leukaemia, and renal cell carcinoma. Such cancers may be KRAS associated cancers. In some embodiments, the cancer comprises a solid tumor. Of particular interest are cancers such as lung cancer, colon cancer, pancreatic cancer, and ovarian cancer. In some embodiment the method of treating cancer is a method of treating chronic myeloid leukaemia. In some embodiments, the cancer comprises a liquid tumor. In some embodiments, the cancer is chronic myeloid leukaemia.

In some embodiments, the additional anticancer agent is an anti-angiogenesis agent, including for example VEGF inhibitors, VEGFR inhibitors, PDGFR inhibitors, sunitinib, bevacizumab, axitinib, SU 14813 (Pfizer), and AG 13958 (Pfizer). Additional anti-angiogenesis agents include sorafenib.

In other embodiments, the additional anti-cancer agent is a so-called signal transduction inhibitor (e.g., inhibiting the means by which regulatory molecules that govern the fundamental processes of cell growth, differentiation, and survival communicated within the cell). Signal transduction inhibitors include small molecules, antibodies, and antisense molecules. Signal transduction inhibitors include for example kinase inhibitors (e.g., tyrosine kinase inhibitors or serine/threonine kinase inhibitors) and cell cycle inhibitors. More specifically signal transduction inhibitors include, for example, farnesyl protein transferase inhibitors, EGF inhibitor, ErbB-1 (EGFR), ErbB-2, pan erb, ERBB family inhibitors, IGF1R inhibitors, MEK, c-Kit inhibitors, Erkl/2 inhibitors, FLT-3 inhibitors, K-Ras inhibitors, PI3 kinase inhibitors, JAK inhibitors, STAT inhibitors, Raf kinase inhibitors, Akt inhibitors, mTOR inhibitor, P70S6 kinase inhibitors, inhibitors of the WNT pathway and so called multi-targeted kinase inhibitors.

In some embodiments, the additional anti-cancer agent is a tyrosine kinase inhibitor. In some embodiments, the tyrosine kinase inhibitor is selected from imatinib and nilotinib.

As noted above, the compounds of the disclosure may be used in combination with one or more additional anti-cancer agents. The efficacy of the compounds of the disclosure in certain tumors may be enhanced by combination with other approved or experimental cancer therapies, e.g., radiation, surgery, chemotherapeutic agents, targeted therapies, agents that inhibit other signaling pathways that are dysregulated in tumors, and other immune enhancing agents, such as PD-1 antagonists and the like.

When a combination therapy is used, the one or more additional anti-cancer agents may be administered sequentially or simultaneously with the compound of the disclosure. In one embodiment, the additional anti-cancer agent is administered to a mammal (e.g., a human) prior to administration of the compound of the disclosure. In another embodiment, the additional anti-cancer agent is administered to the mammal after administration of the compound of the disclosure. In another embodiment, the additional anti-cancer agent is administered to the mammal (e.g., a human) simultaneously with the administration of the compound of the disclosure.

The disclosure also relates to a pharmaceutical composition for the treatment of abnormal cell growth in a mammal, including a human, which comprises an amount of a compound of the disclosure, as defined above (including hydrates, solvates and polymorphs of said compound or pharmaceutically acceptable salts thereof), in combination with one or more (preferably one to three) additional anti-cancer therapeutic agents.

Within the scope of the present disclosure, beneficial or desired clinical results in a subject to which a compound of the disclosure is administered, alone or in the form of a pharmaceutically acceptable composition, include, but are not limited to, one or more of the following: reducing the proliferation of (or destroying) neoplastic or cancerous cell; inhibiting metastasis or neoplastic cells; shrinking or decreasing the size of a tumor; remission of the cancer; decreasing symptoms resulting from the cancer; increasing the quality of life of those suffering from the cancer; decreasing the dose of other medications required to treat the cancer; delaying the progression of the cancer; curing the cancer; overcoming one or more resistance mechanisms of the cancer; and/or prolonging survival of subjects the cancer. Positive therapeutic effects in cancer can be measured in a number of ways (see, for example, W. A. Weber, Assessing tumor response to therapy, J. Nucl. Med. 50 Suppl. 1:1S-10S (2009). For example, with respect to tumor growth inhibition (T/C), according to the National Cancer Institute (NCI) standards, a T/C less than or equal to 42% is the minimum level of anti-tumor activity. A T/C<10% is considered a high anti-tumor activity level, with T/C (%)=median tumor volume of the treated/median tumor volume of the control.times.100.

In some embodiments, the treatment achieved by treatment as disclosed herein is defined by reference to any of the following: partial response (PR), complete response (CR), overall response (OR), progression free survival (PFS), disease free survival (DFS) and overall survival (OS). PFS, also referred to as “Time to Tumor Progression” indicates the length of time during and after treatment that the cancer does not grow and includes the amount of time subjects have experienced a CR or PR, as well as the amount of time subjects have experienced stable disease (SD). DFS refers to the length of time during and after treatment that the subject remains free of disease. OS refers to a prolongation in life expectancy as compared to naive or untreated subjects or subjects. In some embodiments, response to a combination of the disclosure is any of PR, CR, PFS, DFS, OR, or OS that is assessed using Response Evaluation Criteria in Solid Tumors (RECIST) 1.1 response criteria.

The treatment regimen relating to a compound of the disclosure, or a pharmaceutical composition comprising a compound of the disclosure, that is effective to treat cancer in a subject may vary according to factors such as the disease state, age, and weight of the subject, and the ability of the therapy to elicit an anti-cancer response in the subject. While an embodiment of any of the aspects of the disclosure may not be effective in achieving a positive therapeutic effect in every subject, it should do so in a statistically significant number of subjects as determined by any statistical test known in the art such as the Student's t-test, the chi2-test the U-test according to Mann and Whitney, the Kruskal-Wallis test (H-test), Jonckheere-Terpstrat-testy and the Wilcon on-test.

### Dosage Forms and Regimens

Administration of the compounds disclosed herein may be affected by any method that enables delivery of the compounds to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms disclosed herein are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present invention.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-subject dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

The amount of the compound disclosed herein administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.1 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

### Formulations and Routes of Administration

The compounds disclosed herein may be administered to a subject in need thereof in the form of a pharmaceutically acceptable composition comprising one or more compounds disclosed herein and a pharmaceutically acceptable carrier or excipient. The pharmaceutically acceptable carrier may comprise any conventional pharmaceutical carrier or excipient. The choice of carrier and/or excipient will to a large extent depend on factors such as the particular mode of administration, the effect of the carrier or excipient on solubility and stability, and the nature of the dosage form.

Suitable pharmaceutically acceptable carriers include inert diluents or fillers, water and various organic solvents (such as hydrates and solvates). The pharmaceutical compositions may, if desired, contain additional ingredients such as flavorings, binders, excipients, and the like. Thus, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulations, solution suspension, for parenteral injection as a sterile solution, suspension or emulsion, for topical administration as an ointment or cream or for rectal administration as a suppository.

Exemplary parenteral administration forms include solutions or suspensions of active compounds in sterile aqueous solutions, for example, aqueous propylene glycol or dextrose solutions. Such dosage forms may be suitably buffered, if desired.

The pharmaceutical composition may be in unit dosage forms suitable for single administration of precise dosages.

Pharmaceutical compositions suitable for the delivery of compounds disclosed herein and methods for their preparation will be readily apparent to those skilled in the art. Such compositions and methods for their preparation can be found, for example, in ‘Remington's Pharmaceutical Sciences’, 19th Edition (Mack Publishing Company, 1995), the disclosure of which is incorporated herein by reference in its entirety.

The compounds disclosed herein may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds disclosed herein may also be used in fast-dissolving, fast-disintegrating dosage forms such as those described in Expert Opinion in Therapeutic Patents, 11 (6), 981-986 by Liang and Chen (2001), the disclosure of which is incorporated herein by reference in its entirety.

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet. Other conventional ingredients include anti-oxidants, colorants, flavoring agents, preservatives and taste-masking agents. Exemplary tablets contain up to about 80 wt % drug, from about 10 wt % to about 90 wt % binder, from about 0 wt % to about 85 wt % diluent, from about 2 wt % to about 10 wt % disintegrant, and from about 0.25 wt % to about 10 wt % lubricant. Tablet blends may be compressed directly or by roller to form tablets. Tablet blends or portions of blends may alternatively be wet-, dry-, or melt-granulated, melt congealed, or extruded before tableting. The final formulation may include one or more layers and may be coated or uncoated; or encapsulated. The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0-8247-6918-X), the disclosure of which is incorporated herein by reference in its entirety.

Solid formulations for oral administration may be formulated to be immediate and/or modified release. Modified release formulations include delayed-, sustained-, pulsed-, controlled-, targeted and programmed release. Suitable modified release formulations are described in U.S. Pat. No. 6,106,864. Details of other suitable release technologies such as high energy dispersions and osmotic and coated particles can be found in Verma et al, Pharmaceutical Technology On-line, 25(2), 1-14 (2001). The use of chewing gum to achieve controlled release is described in WO 00/35298. The disclosures of these references are incorporated herein by reference in their entireties.

The compounds disclosed herein may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, and subcutaneous. Suitable devices for parenteral administration include needle (including micro needle) injectors, needle-free injectors, and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. The solubility of compounds disclosed herein used in the preparation of parenteral solutions may be increased by the use of appropriate formulation techniques, such as the incorporation of solubility-enhancing agents. Formulations for parenteral administration may be formulated to be immediate and/or modified release. Modified release formulations include delayed-, sustained-, pulsed-, controlled-, targeted and programmed release. Thus, compounds disclosed herein may be formulated as a solid, semi-solid, or thixotropic liquid for administration as an implanted depot providing modified release of the active compound. Examples of such formulations include drug-coated stents and PGLA microspheres.

The compounds disclosed herein may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated; see, for example, J Pharm Sci, 88 (10), 955-958 by Finnin and Morgan (October 1999). Other means of topical administration include delivery by electroporation, iontophoresis, phonophoresis, sonophoresis and micro needle or needle-free (e.g. Powderject™, Bioject™, etc.) injection. The disclosures of these references are incorporated herein by reference in their entireties. Formulations for topical administration may be formulated to be immediate and/or modified release. Modified release formulations include delayed-, sustained-, pulsed-, controlled-, targeted and programmed release.

The compounds disclosed herein can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may include a bioadhesive agent, for example, chitosan or cyclodextrin. The pressurized container, pump, spray, atomizer, or nebulizer contains a solution or suspension of the compound(s) disclosed herein comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid. Prior to use in a dry powder or suspension formulation, the drug product may be micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying. Capsules (made, for example, from gelatin or HPMC), blisters and cartridges for use in an inhaler or insufflator may be formulated to contain a powder mix of the compound disclosed herein, a suitable powder base such as lactose or starch and a performance modifier such as I-leucine, mannitol, or magnesium stearate. The lactose may be anhydrous or in the form of lactose monohydrate, preferably the latter. Other suitable excipients include dextran, glucose, maltose, sorbitol, xylitol, fructose, sucrose and trehalose. A suitable solution formulation for use in an atomizer using electrohydrodynamics to produce a fine mist may contain from 1 μg to 20 mg of the compound disclosed herein per actuation and the actuation volume may vary from 1 μL to 100 μL. A typical formulation includes a compound disclosed herein, propylene glycol, sterile water, ethanol and sodium chloride. Alternative solvents which may be used instead of propylene glycol include glycerol and polyethylene glycol. Suitable flavors, such as menthol and levomenthol, or sweeteners, such as saccharin or saccharin sodium, may be added to those formulations disclosed herein intended for inhaled/intranasal administration. Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release. In the case of dry powder inhalers and aerosols, the dosage unit is determined by means of a valve which delivers a metered amount. Units in accordance with the invention are typically arranged to administer a metered dose or “puff” containing a desired mount of the compound disclosed herein. The overall daily dose may be administered in a single dose or, more usually, as divided doses throughout the day.

Compounds disclosed herein may be administered rectally or vaginally, for example, in the form of a suppository, pessary, or enema. Cocoa butter is a traditional suppository base, but various alternatives may be used as appropriate. Formulations for rectal/vaginal administration may be formulated to be immediate and/or modified release. Modified release formulations include delayed-, sustained-, pulsed-, controlled-, targeted and programmed release.

Compounds disclosed herein may also be administered directly to the eye or ear, typically in the form of drops of a micronized suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis. Formulations for ocular/aural administration may be formulated to be immediate and/or modified release. Modified release formulations include delayed-, sustained-, pulsed-, controlled-, targeted, or programmed release.

Compounds disclosed herein may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubilizer. Most commonly used for these purposes are alpha-, beta- and gamma-cyclodextrins, examples of which may be found in PCT Publication Nos. WO 91/11172, WO 94/02518 and WO 98/55148, the disclosures of which are incorporated herein by reference in their entireties.

### Kit-of-Parts

It is within the scope of the present disclosure that two or more pharmaceutical compositions, at least one of which contains a compound in accordance with the disclosure, may conveniently be combined in the form of a kit suitable for coadministration of the compositions. Thus, the kit disclosed herein includes two or more separate pharmaceutical compositions, at least one of which contains a compound disclosed herein, and means for separately retaining said compositions, such as a container, divided bottle, or divided foil packet. An example of such a kit is the familiar blister pack used for the packaging of tablets, capsules and the like. The kit disclosed herein is particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid.

### EXAMPLES

### Materials and Methods

### General Procedures for Synthesis of Compounds

### Chromatography

Preparative high-pressure liquid chromatography was carried out using apparatus made by Agilent. The apparatus is constructed such that the chromatography is monitored by a multi-wavelength UV detector (G1365B manufactured by Agilent) and an MM-ES+APCI mass spectrometer (G-1956A, manufactured by Agilent) connected in series, and if the appropriate criteria are met the sample is collected by an automated fraction collector (G1364B manufactured by Agilent). Collection can be triggered by any combination of UV or mass spectrometry or can be based on time. Typical conditions for the separation process are as follows: Chromatography column was an Xbridge C-18 (19×100 mm); the gradient was run over a 7 minute period at a flow rate of 40 mL/min (gradient at start: 10% methanol and 90% water, gradient at finish: 100% methanol and 0% water; as buffer: either 0.1% formic acid, 0.1% ammonium hydroxide or 0.1% trifluoroacetic acid was added to the water). It will be appreciated by those skilled in the art that it may be necessary or desirable to modify the conditions for each specific compound, for example by changing the solvent composition at the start or at the end, modifying the solvents or buffers, changing the run time, changing the flow rate and/or the chromatography column.

Flash chromatography refers to silica gel chromatography and carried out using an SP4 or an Isolara 4 MPLC system (manufactured by Biotage); pre-packed silica gel cartridges (supplied by Biotage); or using conventional glass column chromatography.

### Analytical Methods

¹H Nuclear magnetic resonance (NMR) spectroscopy was carried out using an ECX400 spectrometer (manufactured by JEOL) in the stated solvent at around room temperature unless otherwise stated. In all cases, NMR data were consistent with the proposed structures. Characteristic chemical shifts (6) are given in parts-per-million using conventional abbreviations for designation of major peaks: e.g. s, singlet; d, doublet; t, triplet; q, quartet; dd, doublet of doublets; br, broad.

Analytical LCMS was typically carried out using an Agilent HPLC instrument with C-18 Xbridge column (3.5 μm, 4.6×30 mm, gradient at start: 10% organic phase and 90% water, gradient at finish: organic and 0% water; as buffer: either 0.1% ammonium hydroxide or 0.1% trifluoroacetic acid was added to the water). The organic solvent was either acetonitrile or methanol. A flow rate of 3 m/min was used with UV detection at 254 and 210 nm.

Mass spectra were recorded using a MM-ES+APCI mass spectrometer (G-1956A, manufactured by Agilent). Where thin layer chromatography (TLC) has been used it refers to silica gel TLC using silica gel MK6F 60 Å plates, R_{f} is the distance travelled by the compound divided by the distance travelled by the solvent on a TLC plate.

### Compound Preparation

Where the preparation of starting materials is not described, these are commercially available, known in the literature, or readily obtainable by those skilled in the art using standard procedures. Where it is indicated that compounds were prepared analogously to earlier examples or intermediates, it will be appreciated by the skilled person that the reaction time, number of equivalents of reagents, solvent, concentration and temperature can be modified for each specific reaction and that it may be necessary or desirable to employ different work-up or purification techniques.

Where reactions are carried out using microwave irradiation, the microwave used is an Initiator 60 supplied by Biotage. The actual power supplied varies during the course of the reaction in order to maintain a constant temperature.

Some hydrogenations were carried out using an H-Cube® Continuous-flow Hydrogenation Reactor manufactured by ThalesNano. The catalysts are supplied by ThalesNano as cartridges “CatCarts” The pressure, flow rate, temperature and cartridge are indicated in the experimental section. The equipment was used in accordance with the manufacturer operating procedure. The person skilled in the art will appreciate that it may be necessary or desirable to run repeat cycles of the reaction mixture and in some instances, replace the cartridge between cycles to improve the yield of the reaction.

### Abbreviations

A list of some common abbreviations are shown below—where other abbreviations are used which are not listed, these will be understood by the person skilled in the art.
DCM=Dichloromethane
DMF=N,N-Dimethylformamide
THF=Tetrahydrofuran
MeOH=Methanol
TFA=Trifluoroacetic acid
Xantphos=4,5-Bis(diphenylphosphino)-9,9-dimethylxanthene
HATU=N,N,N′,N′-Tetramethyl-O-(7-azabenzotriazol-1-yl)uronium-hexafluorophospate
EDCI=1,3-Propanediamine, N3-(ethylcarbonimidoyl)-N1,N1-dimethyl-, hydrochloride
DCC=1,3-Dicyclohexylcarbodiimide
Pd₂(dba)₃=tris(dibenzylideneacetone)dipalladium(0)
TEA=Triethylamine
rm=Reaction mixture
rt=Room temperature
AcOH=Acetic acid
IPA=Isopropanol
DIPEA=N,N-diisopropylethylamine
TBSMSCl=Tertiarybutyldimethylsilyl chloride
MeCN=Acetonitrile
NH₃=Ammonia
EtOH=Ethanol
EtOAc=Ethyl Acetate
LCMS=Mass spectrometry directed high pressure liquid chromatography
UV=Ultraviolet
SCX=Strong cation exchange
TPAP=Tetrapropylammonium perruthenate
DMSO=Dimethylsulphoxide
BINAP=2,2′-bis(diphenylphosphino)-1,1′-binaphthyl
TPAP=Tetrapropylammonium perruthenate
DIAD=Diisopropyl azodicarboxylate
NMO=N-Methylmorpholine N-oxide

### Synthesis of Compounds

### Intermediate 1: 1-(3-((2-Chloro-5-(trifluoromethyl)pyrimidin-4-yl)amino)propyl)piperidin-2-one

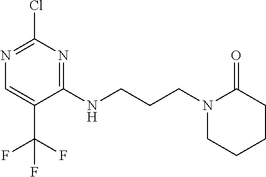 [SMILES: O=C1CCCCN1CCCNc1nc(Cl)ncc1C(F)(F)F]

### Step 1: Synthesis of 3-(2-oxopiperidin-1-yl) propanenitrile

To a stirred solution of piperidin-2-one (10.0 g, 101 mmol) in THF (250 mL) was added 60% NaH (4.8 g, 202 mmol) at 0° C., warmed to room temperature and stirred for 30 min. The reaction mixture was cooled to 0° C., added 2-bromo propionitrile (10.5 mL, 121 mmol) in a dropwise manner for 20 minutes, warmed to room temperature and stirred for 2 h. After completion of reaction (TLC), the reaction mass was quenched with ice water and extracted with ethyl acetate. The organic layer was dried over anhydrous Na₂SO₄, filtered and concentrated. The crude compound was purified by flash column chromatography (SiO₂, 100-200 mesh, 20% ethyl acetate in pet ether) to afford a pale-yellow liquid (8.0 g, 52%). ¹H NMR (400 MHz, CDCl₃): δ 3.58 (t, J=6.8 Hz, 2H), 3.48 (t, J=5.2 Hz, 2H), 2.69 (t, J=6.4 Hz, 2H), 2.40 (t, J=6.8 Hz, 2H), 1.80-1.88 (m, 4H); LCMS (m/z)=153.1 [M+H]⁺

### Step 2: Synthesis of 1-(3-aminopropyl) piperidin-2-one

To a stirred solution 3-(2-oxopiperidin-1-yl) propanenitrile (10.0 g, 65 mmol) in MeOH (200 mL) was added NH₄OH (25 mL) and Ra-Ni (wet) (6.0 g) at rt under nitrogen atmosphere and the resulting reaction mixture stirred under H₂ (balloon) pressure for 16 h. After completion of reaction (TLC), the reaction mixture was filtered through a Celite pad, washing with methanol. The filtrates were combined and concentrated to afford a colorless liquid (8.0 g, 78%). ¹H NMR (400 MHz, DMSO-d₆): δ 3.10-3.31 (m, 5H), 2.44 (q, J=15.2 Hz, 1H), 2.32 (t, J=4.8 Hz, 1H), 2.18 (t, J=6.0 Hz, 2H), 1.83-1.49 (m, 7H); LCMS (m/z)=157.1 [M+H]⁺

### Step 3: Synthesis of 1-(3-((2-chloro-5-(trifluoromethyl)pyrimidin-4-yl)amino)propyl)piperidin-2-one

To a solution of 1-(3-aminopropyl) piperidin-2-one (30 g, 192 mmol) in IPA (300 mL) was added DIPEA (33.4 mL, 192 mmol) at 0° C. and stirred for 10 min. The resulting solution was cooled to −78° C., added a solution of 2, 4-dichloro-5-(trifluoromethyl) pyrimidine (50.0 g, 230 mmol) in IPA (50 mL) and stirred at room temperature for 16 h. After completion of reaction (TLC), the reaction mixture was concentrated, diluted with cold water and extracted with ethyl acetate. The combined organic layers were dried over anhydrous Na₂SO₄, filtered and concentrated. The crude compound was purified by column chromatography (SiO₂, 100-200 mesh, 50% ethyl acetate in pet ether) to afford Intermediate 1 as a light yellow solid (18 g, 28%). ¹H NMR (400 MHz, DMSO-d₆): δ 8.39 (d, J=0.8 Hz, 1H), 8.01 (t, J=5.2 Hz, 1H), 3.41-3.36 (m, 2H), 3.31-3.25 (m, 4H), 2.23 (t, J=6.4 Hz, 2H), 1.75-1.69 (m, 6H); LCMS (m/z)=337.0 [M+H]⁺

### Intermediate 2: 1-(6-Amino-7-chloro-3, 4-dihydroisoquinolin-2(1H)-yl)-2, 2, 2-trifluoroethanone

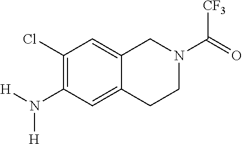 [SMILES: Nc1cc2c(cc1Cl)CN(C(=O)C(F)(F)F)CC2]

### Step 1: Synthesis of N-[2-(4-chlorophenyl)ethyl]-2,2,2-trifluoro-acetamide

To a solution of 2-(4-chlorophenyl)ethanamine (50.0 g, 321 mmol) in DCM (600 mL) was added triethylamine (44.7 mL, 323 mmol) followed by trifluoroacetic anhydride (67.7 g, 322.5 mmol) at 0° C. and the mixture was stirred for 4 h at 0° C. After completion of the reaction (by TLC), the mixture was diluted with DCM, washed with water and brine. The organic phase was dried over anhydrous Na₂SO₄, filtered and concentrated to afford an off-white solid (60.0 g, 75%). ¹H NMR (400 MHz, DMSO-d₆): δ 9.47 (s, 1H), 7.24 (d, J=8.4 Hz, 2H), 7.24 (d, J=8.0 Hz, 2H), 3.38-3.43 (q, J=13.2 & 7.2 Hz, 2H), 2.79 (t, J=7.2 Hz, 2H)

### Step 2: Synthesis of 1-(7-Chloro-3,4-dihydroisoquinolin-2(1H)-yl)-2,2,2-trifluoroethanone

To a solution of N-[2-(4-chlorophenyl)ethyl]-2,2,2-trifluoro-acetamide (50 g, 198 mmol) in acetic acid (107 mL) was added conc. sulfuric acid (71.5 mL) followed by p-formaldehyde (15.8 g, 397.4 mmol) at 0° C., warmed to room temperature and stirred for 4 h. The reaction mixture was poured into ice cold water and extracted with ethyl acetate. The combined organic layer was washed with saturated sodium bicarbonate solution and brine solution, dried over anhydrous Na₂SO₄, filtered, and concentrated to afford compound a viscous oil (45 g, 86%) which was used in the next step without further purification.

### Step 3: Synthesis of 1-(7-Chloro-6-nitro-3, 4-dihydroisoquinolin-2(1H)-yl)-2, 2, 2-trifluoroethanone

To a solution of 1-(7-chloro-3,4-dihydroisoquinolin-2(1H)-yl)-2,2,2-trifluoroethanone (50 g, 190 mmol) in conc. sulfuric acid (500 mL) was added nitric acid (23.9 g, 379 mmol) at −10° C. and stirring was continued for 4 h. The reaction mixture was poured into ice cold water and extracted with ethyl acetate. The combined organic phases were dried over anhydrous Na₂SO₄, filtered and concentrated. The crude compound was purified by flash column chromatography (SiO₂, 100-200 mesh, 10% ethyl acetate in pet ether) to afford a light yellow solid (35 g, 60%). LCMS (m/z)=307.0 [M−H]⁺

### Step 4: Synthesis of 1-(6-Amino-7-chloro-3, 4-dihydroisoquinolin-2(1H)-yl)-2, 2, 2-trifluoroethanone

To a solution of 1-(7-chloro-6-nitro-3,4-dihydroisoquinolin-2(1H)-yl)-2,2,2-trifluoroethanone (14 g, 45.4 mmol) in ethyl acetate (140 mL) was added SnCl₂, 2H₂O (51.2 g, 227 mmol) followed by water (3.5 mL) at room temperature and stirred for 16 h. The reaction mixture was concentrated and purified by flash column chromatography (SiO₂, 100-200 mesh, 10% ethyl acetate in pet ether) to afford Intermediate 2 as a brown solid (5.0 g, 40%). ¹H NMR (400 MHz, DMSO-d₆): δ 7.14 (d, J=12.8 Hz, 1H), 6.59 (d, J=3.2 Hz, 1H), 5.27 (d, J=10.4 Hz, 2H), 4.57 (d, J=8.4 Hz, 2H), 3.75-3.70 (m, 2H), 2.78-2.73 (m, 2H); LCMS (m/z)=279.4 [M+H]⁺

### Intermediate 3: 1-(6-Amino-7-methoxy-3,4-dihydroisoquinolin-2(1H)-yl)-2,2,2-trifluoroethan-1-one

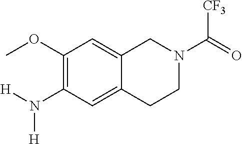 [SMILES: COc1cc2c(cc1N)CCN(C(=O)C(F)(F)F)C2]

### Step 1: Synthesis of 2, 2, 2-trifluoro-N-(4-methoxyphenethyl)acetamide

To a solution of 2-(4-methoxyphenyl)ethan-1-amine (5.0 g, 33 mmol) in DCM (150 mL) was added triethylamine (11.5 mL, 80 mmol) followed by trifluoroacetic anhydride (7.5 g, 35.2 mmol) at 0° C. and stirring was continued at same temperature for 4 h. The reaction mixture was diluted with DCM, washed with water and brine solution. The organic layers were dried over anhydrous Na₂SO₄, filtered, and concentrated to afford an off-white solid (7.1 g, 87%). ¹H NMR (400 MHz, CDCl₃): δ 7.12-7.09 (m, 2H), 6.89-6.85 (m, 2H), 6.27 (brs, 1H), 3.80 (s, 3H), 3.59 (q, J=13.6 & 6.8 Hz, 2H), 2.83 (t, J=7.2 Hz, 2H); LCMS (m/z): 248.3 [M+H]⁺

### Step 2: Synthesis of 2, 2, 2-trifluoro-N-(4-methoxy-3-nitrophenethyl)acetamide

To a solution of 2, 2, 2-trifluoro-N-(4-methoxyphenethyl)acetamide (4.0 g, 16.2 mmol) in TFA (50 mL) was added nitric acid (3.3 mL, 36.1 mmol) at −10° C. and stirring was continued at the same temperature for 2 h. The reaction mixture was poured into ice cold water and extracted with ethyl acetate. The combined organic layers were dried over anhydrous Na₂SO₄, filtered and concentrated. The crude compound was purified by column chromatography (SiO₂, 100-200 mesh, 1% methanol in dichloromethane) to afford a pale yellow solid (3.1 g, 65%). ¹H NMR (400 MHz, CDCl₃): δ 7.70 (d, J=2.4 Hz, 1H), 7.38 (dd, J=2.0 Hz & 8.4 Hz, 1H), 7.06 (d, J=8.8 Hz, 1H), 6.35 (brs, 1H), 3.96 (s, 3H), 3.62 (q, J=13.6 & 6.8 Hz, 2H), 2.91 (t, J=7. Hz, 2H); LCMS (m/z): 293.3 [M+H]⁺

### Step 3: Synthesis of 2, 2, 2-trifluoro-1-(7-methoxy-6-nitro-3,4-dihydroisoquinolin-2(1H)-yl)ethan-1-one

To a solution of 2, 2, 2-trifluoro-N-(4-methoxy-3-nitrophenethyl)acetamide (2.0 g, 6.85 mmol) in acetic acid (13.2 mL) was added conc. sulfuric acid (20 mL) followed by p-formaldehyde (10.3 g, 34.2 mmol) at 50° C. and stirred for 3.5 h at room temperature. The reaction mixture was poured into ice cold water and extracted with ethyl acetate. The combined organic layers were washed with saturated sodium bicarbonate solution followed by brine solution. Organic layer was dried over anhydrous sodium sulfate, filtered and concentrated. The crude compound was purified by preparative HPLC to afford a light yellow solid (1.5 g, 72%). ¹H NMR (400 MHz, DMSO-d₆): δ 7.71 (s, 1H), 7.30 (s, 1H), 4.82 (s, 2H), 3.90 (s, 3H), 3.82 (t, J=5.6 Hz, 2H), 2.91 (s, 2H); LCMS (m/z): 305.3 [M+H]⁺

### Step 4: Synthesis of 1-(6-amino-7-methoxy-3,4-dihydroisoquinolin-2(1H)-yl)-2,2,2-trifluoroethan-1-one

To a solution of 2,2,2-trifluoro-1-(7-methoxy-6-nitro-3,4-dihydroisoquinolin-2(1H)-yl)ethan-1-one (1.5 g, 4.93 mmol) in methanol (200 mL) was added Pd/C (0.3 g) at room temperature and stirred for 2 h under hydrogen (balloon pressure). The reaction mixture was filtered through Celite and washed with methanol and the filtrate was concentrated. Purification by column chromatography (SiO₂, 100-200 mesh, 10% ethyl acetate in pet ether) gave a brown solid (0.580 g, 43%) which was used in the next step without further purification LCMS (m/z): 275.1 [M+H]⁺

### Intermediate 4: 2-Methyl-1,2,3,4-tetrahydroisoquinolin-6-amine

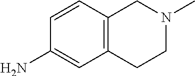

### Step 1: Synthesis of 2-(1,3-dimethoxy-1,3-dioxopropan-2-yl)-4-nitrobenzoic acid

Sodium methoxide (12.9 g, 238 mmol) was slowly added to a slurry of 2-chloro-4-nitrobenzoicacid (10 g, 49.6 mmol) and copper (I) bromide (0.712 g, 4.96 mmol) in dimethyl malonate (113 mL, 992.2 mmol). The resulting reaction mixture was stirred for 15 min at room temperature and subsequently heated at 70° C. with vigorous stirring for 24 h. The reaction mixture was cooled to room temperature and diluted with water (100 mL) and hexane (100 mL). The aqueous layer was separated and toluene (100 mL) was added. The mixture was filtered through the Celite, washed with hexane (100 mL) and toluene (100 mL). The resulting biphasic mixture was acidified to pH 1 with 6N HCl solution, the precipitate was filtered, washed with toluene (150 mL) and hexane (150 mL) and dried in vacuum to afford a white solid (3 g, 20%). ¹H NMR (300 MHz, DMSO-d₆): δ 13.8 (br s, 1H), 8.32 (dd, J₁=2.4 Hz, J₂=8.7 Hz, 1H), 8.22-8.15 (m, 2H), 5.81 (s, 1H), 3.69 (s, 6H); LCMS (m/z): 298.30 (M+H)+

### Step 2: Synthesis of 2-(carboxymethyl)-4-nitrobenzoic acid

A solution of sodium hydroxide (16.1 g, 403 mmol) in water (120 mL) was added over 90 min to a solution of 2-(1,3-dimethoxy-1,3-dioxopropan-2-yl)-4-nitrobenzoic acid (24 g, 80.74 mmol) in methanol (120 mL) at room temperature and the mixture was stirred for 3 h. The mixture was concentrated under reduced pressure, and the residue was acidified with concentrated HCl (22.4 mL) at room temperature. The resulting white aqueous suspension was extracted with ethyl acetate (2×250 mL), the combined organic layers were dried over anhydrous sodium sulphate, filtered and the filtrate volume was reduced to 100 mL. The resulting solution was heated to 70° C. for 6 h and the precipitated solid was filtered and washed with ethyl acetate, dried in vacuum to afford a white solid (13 g, 71%). ¹H NMR (300 MHz, DMSO-d₆): δ 13.8-12.4 (br s, 2H), 8.27 (d, J=2.1 Hz, 1H), 8.22-8.19 (m, 1H), 8.09 (d, J=9.0 Hz, 1H), 4.09 (s, 2H).

### Step 3: Synthesis of 2-(2-(hydroxymethyl)-5-nitrophenyl)ethan-1-ol

To a solution of 2-(carboxymethyl)-4-nitrobenzoic acid (13 g, 57.7 mmol) in THF (60 mL) was added 1M borane in THF (289 mL, 289 mmol) at 0° C. The resulting reaction mixture was allowed to warm to room temperature and then heated at 70° C. for 3 h. The reaction mixture was cooled to room temperature then 0° C., quenched with methanol and the resulting reaction mixture was heated to reflux for 2 h. The reaction mixture was concentrated; residue was partitioned between ethyl acetate (300 mL) and water (150 mL). The organic layer was dried over anhydrous sodium sulfate, filtered, and concentrated under reduced pressure to afford an off-white solid (9 g, 79%). ¹H NMR (400 MHz, D₂O): δ 8.07-8.06 (m, 2H), 7.68 (d, J=8.4 Hz, 1H), 4.65 (s, 2H), 3.67-3.63 (m, 2H), 2.83 (t, J=6.4 Hz, 2H)

### Step 4: Synthesis of 2-(2-((methylsulfonyl)oxy)ethyl)-4-nitrobenzyl methanesulfonate

To a solution of 2-(2-(hydroxymethyl)-5-nitrophenyl) ethan-1-ol (4 g, 20.3 mmol) in dry DCM (40 mL) at 0° C. was added triethylamine (8.4 mL, 60.8 mmol) and methanesulfonyl chloride (5.8 g, 50.7 mmol and stirring was continued at the same temperature for 30 minutes. The reaction mixture was diluted with dichloromethane (100 mL), washed with water (50 mL) and saturated NaHCO₃ (aq) (40 mL). The organic layer was dried over anhydrous sodium sulfate, filtered and concentrated under reduced pressure to afford an off-white solid (4 g, 56%). (m/z): 376.25 (M+Na)⁺

### Step 5: Synthesis of 2-methyl-6-nitro-1,2,3,4-tetrahydroisoquinoline

To a solution of 2-(2-((methylsulfonyl)oxy)ethyl)-4-nitrobenzyl methanesulfonate (4 g, 11.31 mmol) in THF (20 mL) was added methyl amine (2M in THF) (40 mL) at 0° C., the mixture was allowed to room temperature and then heated at 100° C. for 16 h in a sealed tube. The reaction mixture was cooled to room temperature and concentrated; residue was purified flash column chromatography using silica gel 100-200 mesh (gradient elution from 40-50% EtOAc/Petroleum ether) to give a white solid (1.5 g, 69%). ¹H NMR (400 MHz, CDCl₃): δ 7.99-7.95 (m, 2H), 7.16 (d, J=8.4 Hz, 1H), 3.65 (s, 2H), 3.01 (t, J=6.0H, 2H), 2.72 (t, J=6.0 Hz, 2H), 2.48 (s, 3H).

### Step 6: Synthesis of 2-methyl-1,2,3,4-tetrahydroisoquinolin-6-amine

A Parr-shaker vessel was charged with a solution of 2-methyl-6-nitro-1,2,3,4-tetrahydroisoquinoline (1.5 g, 7.80 mmol) in methanol (45 mL) and 10% Pd/C (200 mg). The mixture was hydrogenated at 50 psi for 2 h. The reaction mixture was filtered through Celite pad, washed with methanol, and the filtrate was concentrated to afford a brown gel (1.2 g, 95%). ¹H NMR (400 MHz, CDCl₃): δ 6.80 (d, J=8.4 Hz, 1H), 6.49-6.44 (m, 2H), 3.51 (brs, 2H), 3.46 (s, 2H), 2.82 (t, J=6.0 Hz, 2H), 2.63 (t, J=6.0 Hz, 2H), 2.42 (s, 3H); LCMS (m/z): 163.40 (M+H)⁺

### Intermediate 5: 2-Methyl-1,2,3,4-tetrahydroisoquinolin-7-amine

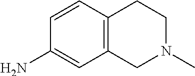

### Step 1: Synthesis of 7-nitro-1,2,3,4-tetrahydroisoquinoline

1,2,3,4-Tetrahydroisoquinoline (10 g, 75.08 mmol) was added dropwise to a stirred ice-cold solution of concentrated H₂SO₄ (37.5 mL). Then KNO₃ (8.34 g, 82.58 mmol) was added portion wise while maintaining the temperature below 5° C. The resulting reaction mixture was stirred at room temperature for a further 16 h. The reaction mixture was carefully poured onto an ice-cold solution of concentrated ammonium hydroxide, and then extracted with chloroform. The combined organic layers were washed with brine, dried over anhydrous sodium sulfate, filtered and concentrated under reduced pressure. The resulting dark brown oil was taken up into ethanol, cooled in an ice bath and treated with concentrated HCl. The yellow precipitate was collected via filtration and recrystallized from methanol to give a yellow solid (4 g, 30%). ¹H NMR (400 MHz, DMSO-d₆): δ 9.69 (brs, 1H), 8.21 (d, J=2.0 Hz, 1H), 8.11 (dd, J₁=2.4, J₂=8.4 Hz, 1H), 7.52 (d, J=8.8 Hz, 1H), 4.38 (s, 2H), 3.39 (t, J=6.4 Hz, 2H), 3.14 (t, J=6.4 Hz, 2H); LCMS (m/z): 179.34 (M+H)⁺

### Step 2: Synthesis of 2-methyl-7-nitro-1,2,3,4-tetrahydroisoquinoline

To a mixture of formaldehyde (8.2 mL) and formic acid (5 mL) was added 7-nitro-1,2,3,4-tetrahydroisoquinoline (2 g, 11.22 mmol) and the mixture was heated at 100° C. for 4 h. The reaction was cooled to room temperature, poured into ice, and basified to pH 11 with aqueous ammonia solution, and the gummy residue which precipitated was extracted with dichloromethane (2×150 mL). The combined organic extracts were dried over anhydrous sodium sulfate, filtered and concentrated under reduced pressure. Purified by flash column chromatography using flurosil (eluent: 0-5% HCl-DCM gave a black solid (900 mg, 42%). ¹H NMR (400 MHz, DMSO-d₆): δ 7.99-7.97 (m, 2H), 7.39 (d, J=9.2 Hz, 1H), 3.59 (s, 2H), 2.93 (t, J=6.0 Hz, 2H), 2.62 (t, J=6.0 Hz, 2H), 2.35 (s, 3H); LCMS (m/z): 193.45 (M+H)⁺

### Step 3: Synthesis of 2-methyl-1,2,3,4-tetrahydroisoquinolin-7-amine

To a Parr shaker vessel charged with a solution of 2-methyl-7-nitro-1,2,3,4-tetrahydroisoquinoline (1 g, 5.20 mmol) in methanol (45 mL) was added 10% Pd/C (100 mg) under nitrogen atmosphere at room temperature. The mixture was hydrogenated at 50 psi for 3 h. The reaction mixture was filtered through a Celite pad, washed with methanol and the filtrate was concentrated to afford a pale yellow solid (600 mg, 71%). ¹H NMR (300 MHz, DMSO-d₆): δ 6.72 (d, J=8.1 Hz, 1H), 6.35 (dd, J₁=2.1 Hz, J₂=8.1 Hz, 1H), 6.21 (d, J=1.8 Hz, 1H), 4.75 (s, 2H), 3.30 (s, 2H), 2.61 (t, J=6.0 Hz, 2H), 2.50-2.46 (m, 2H), 2.28 (s, 3H); LCMS (m/z): 163.26 (M+H)⁺

### Intermediate 6: 2-Chloro-N-(3-(pyrrolidin-1-yl)propyl)-5-(trifluoromethyl)pyrimidin-4-amine

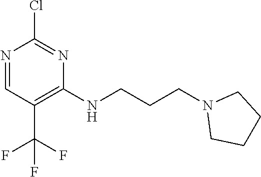 [SMILES: FC(F)(F)c1cnc(Cl)nc1NCCCN1CCCC1]

To a solution of 2,4-dichloro-5-(trifluoromethyl)pyrimidine (1 g, 4.60 mmol) in IPA (20 mL) was added DIPEA (2.4 mL, 13.82 mmol) and 3-(pyrrolidin-1-yl)propan-1-amine (0.88 g, 6.91 mmol) at 0° C. and the mixture was allowed to stir at room temperature and then heated at 50° C. for 16 h. The reaction mixture was evaporated; the residue was taken in ethyl acetate (40 mL) and washed with water (40 mL), organic layer was dried over anhydrous sodium sulfate, filtered and the filtrate was concentrated. The crude was purified by flash column chromatography (silica gel, 100-200 mesh, eluted with 50% ethyl acetate/pet ether) to afford an off-white solid (250 mg, 18%). ¹H NMR (400 MHz, CDCl₃): δ 8.99 (brs, 1H), 8.17 (s, 1H), 3.23 (q, J=5.6 Hz, 2H), 2.70 (t, J=5.6 Hz, 2H), 2.55 (brs, 4H), 1.80-1.76 (m, 6H); LCMS (m/z): 309.39 (M+H)⁺

### Intermediate 7: 1-(3-(2-Chloro-5-cyclopropylpyrimidin-4-ylamino) propyl) pyrrolidin-2-one

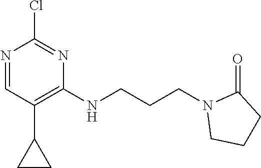 [SMILES: O=C1CCCN1CCCNc1nc(Cl)ncc1C1CC1]

To a stirred solution of 2,4-dichloro-5-cyclopropylpyrimidine (400 mg, 2.11 mmol) in IPA (5 mL) was added DIPEA (0.55 mL, 3.16 mmol) and 1-(3-aminopropyl)pyrrolidin-2-one (0.33 mL, 2.32 mmol) at 0° C., and the reaction mixture was heated at 50° C. for 16 h. The mixture was concentrated under reduced pressure and the residue was partitioned between water (20 mL) and ethyl acetate (30 mL). The aqueous layer was extracted with ethyl acetate (2×30 mL), the combined organic layers were washed with brine (30 mL), dried over anhydrous Na₂SO₄, filtered and concentrated. The crude compound was purified by automated flash chromatography (gradient elution from 0-5% MeOH/DCM) to afford a white solid (350 mg, 56%). ¹H NMR (300 MHz, CDCl₃): δ 7.73 (s, 1H), 6.95-6.86 (m, 1H), 3.53-3.37 (m, 6H), 2.46 (t, J=8.1 Hz, 2H), 2.14-2.04 (m, 2H), 1.81-1.73 (m, 2H), 1.53-1.49 (m, 1H), 1.05-0.98 (m, 2H), 0.58-0.52 (m, 2H); LCMS (m/z): 295.47 [M+H]⁺.

### Intermediate 8: 1-(3-((2-Chloro-5-cyclopropylpyrimidin-4-yl)amino)propyl)piperidin-2-one

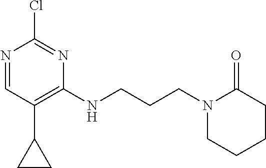 [SMILES: O=C1CCCCN1CCCNc1nc(Cl)ncc1C1CC1]

To a stirred solution of 2,4-dichloro-5-cyclopropylpyrimidine (300 mg, 1.58 mmol) in IPA (5 mL) at 0° C. was added DIPEA (0.83 mL, 4.74 mmol) and 1-(3-aminopropyl)piperidin-2-one. TFA salt (602 mg, 2.37 mmol), and stirring was continued at room temperature for 5 h. The reaction mixture was evaporated; the residue was taken up in ethyl acetate (20 mL) and washed with water (20 mL). The organic layer was dried over anhydrous sodium sulfate, filtered and concentrated. Purification by flash column chromatography (silica gel, 100-200 mesh, eluted with 20% ethyl acetate/pet ether) gave an off-white solid (200 mg, 41%). ¹H NMR (400 MHz, CDCl₃): δ 7.72 (s, 1H), 7.10 (brs, 1H), 3.51-3.46 (m, 4H), 3.30 (t, J=5.6 Hz, 2H), 2.43 (t, J=5.6 Hz, 2H), 1.87-1.73 (m, 6H), 1.57-1.50 (m, 1H), 1.04-0.99 (m, 2H), 0.58-0.52 (m, 2H); LCMS (m/z): 309.43 (M+H)⁺

### Intermediate 9: 4-(4-Aminophenyl)-1-methylpiperidin-4-ol

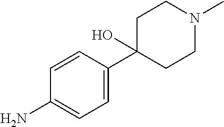

### Step 1: Synthesis of 4-(4-bromophenyl)-1-methylpiperidin-4-ol

To a solution of 1,4-dibromobenzene (15 g, 63.58 mmol) in dry tetrahydrofuran (150 mL) was added n-BuLi (2.5 M, in n-hexane, 28 mL, 69.93 mmol) under nitrogen atmosphere at −78° C. and the mixture was stirred at the same temperature for 30 minutes. N-methyl 4-piperidone 19 (7.91 g, 69.93 mmol) was added at −78° C. and the resulting reaction mixture was stirred at −78° C. for 2 h. The reaction mixture was quenched with saturated NH₄Cl (aq) (30 mL) and diluted with ethyl acetate (100 mL). The organic layer was washed with brine (50 mL), dried over anhydrous sodium sulfate, filtered and concentrated under reduced pressure to afford an off-white solid (6 g, 35%). ¹H NMR (300 MHz, DMSO-d₆): δ 7.50-740 (m, 4H), 4.86 (s, 1H), 2.56-2.48 (m, 2H), 2.37-2.28 (m, 2H), 2.19 (s, 3H), 1.90 (dt, J₁=4.2, 12.6 Hz, 2H), 1.54 (d, J=11.4 Hz, 2H).

### Step 2: Synthesis of 4-(4-((diphenylmethylene)amino)phenyl)-1-methylpiperidin-4-ol

A mixture of 4-(4-bromophenyl)-1-methylpiperidin-4-ol (3 g, 11.10 mmol), benzophenone imine (2.41 g, 13.3 mmol), cesium carbonate (10.8 g, 33.3 mmol), in 1,4-dioxane (60 mL) was degassed with argon for 5 minutes, prior to addition of Xantphos (0.81 g, 0.88 mmol) and Pd₂(dba)₃ (0.60 g, 0.66 mmol). The resulting reaction mixture was heated at 100° C. for 16 h, cooled to room temperature filtered through a celite pad, washed with ethyl acetate. The filtrate was concentrated and the residue was purified by flash column chromatography (gradient elution from 2-5% MeOH in DCM) to give an off-white solid. ¹H NMR (300 MHz, DMSO-d₆): δ 7.62 (dd, J₁=1.8, J₂=8.4 Hz, 1H), 7.52-7.42 (m, 4H), 7.33-7.31 (m, 3H), 7.23 (d, J=8.4 Hz, 2H), 7.16-7.13 (m, 2H), 6.63 (d, J=8.7 Hz, 2H), 4.61 (s, 1H), 2.49-2.48 (m, 2H), 2.32-2.25 (m, 2H), 2.16 (s, 3H), 1.88-1.78 (m, 2H), 1.48 (d, J=11.7 Hz, 2H); LCMS (m/z): 371.4 (M+H)⁺

### Step 3: Synthesis of 4-(4-aminophenyl)-1-methylpiperidin-4-ol

A mixture of 4-(4-((diphenylmethylene)amino)phenyl)-1-methylpiperidin-4-ol 22 (1.5 g, 4.05 mmol), sodium acetate (0.83 g, 10.13 mmol) and hydroxylamine hydrochloride (0.5 g, 7.47 mmol) in methanol (30 mL) was stirred at room temperature for 2 h. The mixture was concentrated and diluted with dichloromethane and 0.1 M NaOH solution. The organic layers were combined, dried over anhydrous sodium sulfate, filtered and concentrated. Purification by flash chromatography (gradient elution from 2-5% MeOH in DCM) gave a pale yellow solid (0.3 g, 36%). LCMS (m/z): 207.42 (M+H)⁺

### Intermediate 10: 2-Chloro-N,5-dicyclopropylpyrimidin-4-amine

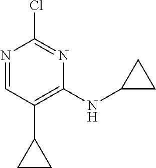 [SMILES: Clc1ncc(C2CC2)c(NC2CC2)n1]

To a solution of 2,4-dichloro-5-cyclopropylpyrimidine (0.5 g, 2.65 mmol) in IPA (5 mL) was added DIPEA (0.68 mL, 3.98 mmol) and cyclopropylamine (0.18 g, 3.19 mmol) and the mixture was stirred at 50° C. for 16 h. The reaction mixture was evaporated; the residue was taken in ethyl acetate (20 mL) and washed with water (20 mL) and the organic phase was dried over anhydrous sodium sulfate, filtered and concentrated. Purification by flash column chromatography (eluent: 20% ethyl acetate/pet ether) gave a white solid (0.4 g, 72%). ¹H NMR (300 MHz, DMSO-d₆): δ ppm 7.68 (d, J=0.9 Hz, 1H), 7.41 (d, J=2.7 Hz, 1H), 2.86-2.80 (m, 1H), 1.57-1.51 (m, 1H), 0.88-0.82 (m, 2H), 0.78-0.72 (m, 2H), 0.63-0.52 (m, 4H). LCMS (m/z): 210.39 [M+H]⁺.

### Intermediate 11: Tert-butyl 7-amino-6-methoxy-3,4-dihydro-1H-isoquinoline-2-carboxylate

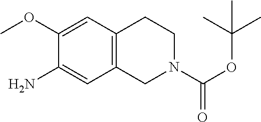 [SMILES: COc1cc2c(cc1N)CN(C(=O)OC(C)(C)C)CC2]

### Step 1: Ethyl N-[2-(3-fluorophenyl)ethyl]carbamate

To 3-fluorophenethyl amine (1.0 g, 7.19 mmol) in Et₃N (2.08 mL, 1.43 mmol) was added ethylchloroformate (0.82 mL, 8.63 mmol) dropwise, and the solution left to stir and warm to room temperature over 2.5 hours. The reaction mixture was concentrated in vacuo to give a white solid. This was triturated with EtOAc, the solid removed via vacuum filtration and the filtrate concentrated in vacuo to give the product, ethyl N-[2-(3-fluorophenyl)ethyl]carbamate as a yellow oil (1.45 g, 68.7 mmol, 96%). ¹H NMR (400 MHz, CHLOROFORM-d) d 7.23-7.30 (m, 1H), 6.97 (d, J=7.33 Hz, 1H), 6.87-6.95 (m, 2H), 4.53-4.78 (m, 1H), 4.11 (q, J=7.02 Hz, 2H), 3.43 (q, J=6.87 Hz, 2H), 2.81 (t, J=7.10 Hz, 2H), 1.18-1.28 (m, 3H).

### Step 2: 6-Fluoro-3,4-dihydro-2H-isoquinolin-1-one

Ethyl N-[2-(3-fluorophenyl)ethyl]carbamate (1.45 g, 68.7 mmol) and polyphosphoric acid (10 mL) were combined and stirred at 120° C. for 2 hours. The reaction mixture was cooled to 70° C. and added to stirring water. EtOAc was added and the organic layer extracted, dried and concentrated in vacuo onto silica. The compound was purified via column chromatography (40-80% EtOAc in PE). The fractions containing product were combined and concentrated to give a colorless oil (0.367 g, 33%). ¹H NMR (400 MHz, CHLOROFORM-d) d 8.07 (dd, J=5.95, 8.70 Hz, 1H), 6.99-7.10 (m, 2H), 6.91 (dd, J=2.52, 8.93 Hz, 1H), 3.58 (dt, J=2.75, 6.64 Hz, 2H), 2.99 (t, J=6.64 Hz, 2H); LCMS (m/z): 166.0 [M+H]⁺.

### Step 3: 6-Fluoro-7-nitro-3,4-dihydro-2H-isoquinolin-1-one

To a solution of 6-fluoro-3,4-dihydro-2H-isoquinolin-1-one (0.367 g, 2.22 mmol) in H₂SO₄ at 0° C. was added dropwise HNO₃ (0.11 mL, 2.67 mmol) and left to stir for 45 minutes at 0° C. The reaction mixture was added to water and extracted with EtOAc. The organic layer was washed with NaHCO₃ (aq.), dried and concentrated in vacuo to give a yellow solid (0.334 g, 72%). ¹H NMR (400 MHz, CHLOROFORM-d) d 8.80 (d, J=7.50 Hz, 1H), 7.15-7.21 (m, 1H), 6.25-6.39 (m, 1H), 3.66 (dt, J=2.70, 6.70 Hz, 2H), 3.12 (t, J=6.40 Hz, 2H).

### Step 4: 6-Methoxy-7-nitro-3,4-dihydro-2H-isoquinolin-1-one

To a solution of 6-fluoro-7-nitro-3,4-dihydro-2H-isoquinolin-1-one (0.100 g, 0.48 mmol) in MeOH (15 mL) was added NaOMe (0.027 g, 0.50 mmol) and heated at reflux overnight, cooled and concentrated via vacuum filtration to give a dark yellow solid (0.110 g). ¹H NMR (400 MHz, CHLOROFORM-d) d 8.57 (s, 1H), 6.90 (s, 1H), 6.25-6.33 (m, 1H), 4.02 (s, 3H), 3.60-3.65 (m, 2H), 3.05-3.10 (m, 2H).

### Step 5: 6-Methoxy-7-nitro-1,2,3,4-tetrahydroisoquinoline

To a solution of 6-methoxy-7-nitro-3,4-dihydro-2H-isoquinolin-1-one (0.110 g, 0.50 mmol) in THF (10 mL) was added 1M BH₃ in THF (0.99 μL, 0.99 mmol), and the reaction mixture heated at reflux overnight. The reaction mixture was cooled, concentrated, extracted with EtOAc and washed with saturated NaHCO₃. The organic layer was separated, dried and concentrated in vacuo. The residue was taken up in DCM/MeOH, passed through an SCX cartridge and the product eluted with 2M NH₃ in MeOH. The eluent was concentrated in vacuo to give the product, 6-methoxy-7-nitro-1,2,3,4-tetrahydroisoquinoline (0.027 g, 26%). ¹H NMR (400 MHz, CHLOROFORM-d) d 7.60 (s, 1H), 6.79 (s, 1H), 4.00 (s, 2H), 3.94 (s, 3H), 3.14-3.18 (m, 2H), 2.83-2.89 (m, 2H); LCMS (m/z): 209.0 [M+H]⁺.

### Step 6: Tert-butyl 6-methoxy-7-nitro-3,4-dihydro-1H-isoquinoline-2-carboxylate

To a solution of 6-methoxy-7-nitro-1,2,3,4-tetrahydroisoquinoline (0.027 g, 0.13 mmol) and Et₃N (28 μL, 0.20 mmol) in DCM (50 mL) was added BOC₂O (1.91 g, 8.77 mmol) followed by DMAP (4 mg, 0.033 mmol) and the resulting mixture left to stir overnight. DCM and H₂O were added, the organic layer separated, dried and concentrated in vacuo onto silica. The compound was purified via column chromatography (5-30% EtOAc in PE). The fractions containing the product were combined and concentrated in vacuo to give a yellow gum (0.029 g, 72%). ¹H NMR (400 MHz, CHLOROFORM-d) d 7.68 (s, 1H), 6.84 (s, 1H), 4.55 (s, 2H), 3.94 (s, 3H), 3.63-3.71 (m, 2H), 2.84-2.92 (m, 2H), 1.50 (s, 9H); LCMS (m/z): 208.9 [M-CO₂^{t}Bu+H]⁺.

### Step 7: Tert-butyl 7-amino-6-methoxy-3,4-dihydro-1H-isoquinoline-2-carboxylate

A solution of tert-butyl 6-methoxy-7-nitro-3,4-dihydro-1H-isoquinoline-2-carboxylate (0.029 g, 0.094 mmol) in EtOAc (50 mL) and MeOH (50 mL) was passed through the H-Cube reactor fitted with a 10% Pd/C cartridge, cf 1 m/min, 25° C., under ‘Full H₂’. The solvent was evaporated yielding the product, (0.014 g, 54%). ¹H NMR (400 MHz, CHLOROFORM-d) d 6.52-6.59 (m, 2H), 4.44 (s, 2H), 3.84 (s, 3H), 3.57-3.66 (m, 2H), 2.69-2.78 (m, 2H), 1.48 (s, 9H); LCMS (m/z): 177.0 [M-CO₂^{t}Bu+H]⁺.

### Intermediate 12: Tert-butyl 7-amino-6-ethoxy-3,4-dihydro-1H-isoquinoline-2-carboxylate

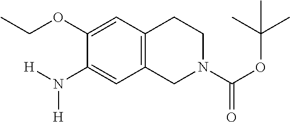 [SMILES: CCOc1cc2c(cc1N)CN(C(=O)OC(C)(C)C)CC2]

Prepared analogously to Intermediate 11 except sodium ethoxide was used instead of sodium methoxide in step 4.

### Intermediate 13: Isochroman-7-amine

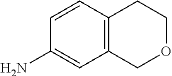 [SMILES: Nc1ccc2c(c1)COCC2]

### Step 1: 2-(carboxymethyl)-5-nitro-benzoic acid

To a suspension of homophthalic acid (1.0 g, 5.55 mmol) in H₂SO₄ (2.5 mL), at 0° C., was added HNO₃ (0.28 mL, 6.66 mmol), and the mixture left to stir for 2 hours. The reaction mixture was added to water and extracted with EtOAc, the organic layer separated, dried and concentrated in vacuo to give a yellow solid. Trituration with Et₂O yielded a white solid (0.605 g, 48%). ¹H NMR (400 MHz, DMSO-d6) d 8.61 (d, J=2.75 Hz, 1H), 8.36 (dd, J=2.40, 8.40 Hz, 1H), 7.67 (d, J=8.40 Hz, 1H), 4.10 (s, 2H).

### Step 2: 7-nitroisochromane

To a solution of 2-(carboxymethyl)-5-nitro-benzoic acid (0.610 g, 2.70 mmol) in THF (20 mL) was added 1M BH₃ in THF (8.1 mL, 8.10 mmol) and the reaction mixture refluxed at 70° C. for 2 hours. The reaction mixture was cooled, and 1M BH₃ in THF (5.4 mL, 5.40 mmol) was added, followed by reflux for 24 hours. The reaction mixture was cooled, followed by addition of 2M HCl (aq.) (6 mL) and heating at 70° C. for 30 minutes. The reaction mixture was cooled, diluted with EtOAC and neutralized with saturated NaHCO₃ (aq.). The organic layer was separated, dried and concentrated in vacuo onto silica. The compound was purified via column chromatography (10-100% EtOAc in petroleum ether) to 7-nitro-isochromane (0.060 g, 12%). ¹H NMR (400 MHz, DMSO-d6) d 8.03 (dd, J=2.30, 8.47 Hz, 1H), 7.99 (d, J=2.29 Hz, 1H), 7.43 (d, J=8.40 Hz, 1H), 4.78 (s, 2H), 3.90 (t, J=5.72 Hz, 2H), 2.91 (t, J=5.72 Hz, 2H).

### Step 3: Isochroman-7-amine

A solution of 7-nitroisochromane (0.040 g, 0.279 mmol) in EtOAc (2 mL) and MeOH (8 mL) was passed through the H-Cube reactor, fitted with a PtO₂ cartridge at 1 m/min, 25° C., under ‘Full H₂’. To ensure maximal conversion to product, the reaction mixture was passed through for a second time under the same conditions. The resulting solution was concentrated in vacuo to give a white solid (0.030 g, 0.201 mmol, 71% Yield). ¹H NMR (400 MHz, DMSO-d6) d 6.76 (d, J=8.10 Hz, 1H), 6.38 (dd, J=2.40, 8.00 Hz, 1H), 6.18 (d, J=2.40 Hz, 1H), 4.86 (br. s, 2H), 4.51 (s, 2H), 3.79 (t, J=5.60 Hz, 2H), 2.58 (t, J=5.50 Hz, 2H); LCMS (m/z): 150.1 [M+H]⁺.

### Intermediate 14: 1-(4-Amino-3-methoxy-pyrazol-1-yl)-2-methyl-propan-2-ol

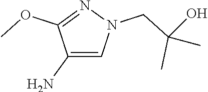

### Step 1: 1-(3-methoxy-4-nitro-pyrazol-1-yl)-2-methyl-propan-2-ol

To 3-methoxy-4-nitro-1H-pyrazole (1.0 g, 7.87 mmol) and Cs₂CO₃ (7.67 g, 23.6 mmol) in DMF (40 mL) was added isobutylene oxide (1.4 mL, 15.7 mmol) and the reaction heated at 100° C. for 3 hours. The reaction mixture was cooled, diluted with EtOAc and H₂O, the organic layer separated, washed with H₂O/LiCl (2×), separated, dried and concentrated in vacuo to give a light yellow solid. The solid was absorbed onto silica and purified via column chromatography (40-60% EtOAc in PE). The fractions containing the desired product were combined and concentrated to afford a light yellow solid (0.800 g, 47%).

### Step 2: 1-(4-amino-3-methoxy-pyrazol-1-yl)-2-methyl-propan-2-ol

A solution of 1-(3-methoxy-4-nitro-pyrazol-1-yl)-2-methyl-propan-2-ol (0.100 g, 0.436 mmol) in IPA (2 mL) was passed through the H-Cube reactor fitted with a Pd/C cartridge, at 1 mL/min, 25° C., under ‘Full H₂’. The solution was concentrated in vacuo to yield the product, 1-(4-amino-3-methoxy-pyrazol-1-yl)-2-methyl-propan-2-ol (0.076 g, 0.411 mmol, 94% Yield). LCMS (m/z): 186.1 [M+H]⁺.

### Intermediate 15: 7-Amino-6-methoxy-3,4-dihydro-2H-isoquinolin-1-one

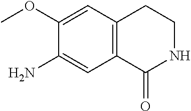

A solution of 6-methoxy-7-nitro-3,4-dihydro-2H-isoquinolin-1-one (0.133 g, 0.599 mmol) in IPA (2 mL) was passed through the H-Cube reactor fitted with a Pd/C cartridge, at 1 mL/min, 25° C., under ‘Full H₂’ twice. The solution was concentrated in vacuo to yield the 7-amino-6-methoxy-3,4-dihydro-2H-isoquinolin-1-one (0.093 g, 81%). ¹H NMR (400 MHz, CHLOROFORM-d) d 7.40 (s, 1H), 6.58 (s, 1H), 6.32 (br. s, 1H), 3.90 (s, 3H), 3.53 (dt, J=2.75, 6.64 Hz, 2H), 2.89 (t, J=6.60 Hz, 2H); LCMS (m/z): 193.0 [M+H]⁺.

### Intermediate 16: 6-Amino-5-methoxy-2-methyl-isoindolin-1-one

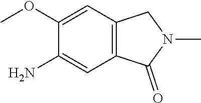

### Step 1: Methyl 4-fluoro-2-methyl-benzoate

To a solution of 4-fluoro-2-methyl benzoic acid (2.5 g, 16.2 mmol) in MeOH (100 mL) was added H₂SO₄ (5 mL) and the reaction mixture heated at 70° C. overnight. The reaction mixture was cooled, diluted with DCM and H₂O, the organic layer separated, washed with NaHCO₃ (aq.), dried and concentrated in vacuo to a brown oil (1.0 g, 37%). ¹H NMR (400 MHz, CHLOROFORM-d) d 7.96 (dd, J=6.18, 8.47 Hz, 1H), 6.89-6.97 (m, 2H), 3.89 (s, 3H), 2.61 (s, 3H).

### Step 2: Methyl 4-fluoro-2-methyl-5-nitro-benzoate

To methyl 4-fluoro-2-methyl-benzoate (1.0 g, 6.49 mmol) in H₂SO₄ (10 mL), at 5° C., was added HNO₃ (0.30 mL, 7.14 mmoL) dropwise and left to stir for 45 minutes. The reaction mixture was poured into H₂O and extracted with EtOAc. The organic layer was separated, dried and concentrated in vacuo to give a yellow oil (1.22 g, 95% Yield). ¹H NMR (400 MHz, CHLOROFORM-d) d 8.71 (d, J=8.00 Hz, 1H), 7.20 (d, J=11.70 Hz, 1H), 3.95 (s, 3H), 2.71 (s, 3H).

### Step 3: Methyl 4-methoxy-2-methyl-5-nitro-benzoate

To methyl 4-fluoro-2-methyl-5-nitro-benzoate (1.22 g, 6.12 mmol) in MeOH (50 mL) was added NaOMe (0.347 g, 6.43 mmol) and the reaction mixture heated at reflux for 4 hours. The reaction mixture was cooled, water and EtOAc added, the organic layer separated, dried and concentrated in vacuo onto silica. The compound was purified via column chromatography (5-10% EtOAc in PE) to yield a white solid (0.741 g, 57%). ¹H NMR (400 MHz, CHLOROFORM-d) d 8.58 (s, 1H), 6.93 (s, 1H), 4.02 (s, 3H), 3.91 (s, 3H), 2.72 (s, 3H).

### Step 4: Methyl 2-(bromomethyl)-4-methoxy-5-nitro-benzoate

To methyl 4-methoxy-2-methyl-5-nitro-benzoate (0.741 g, 3.51 mmol) in MeCN (20 mL) was added NBS (0.750 g, 4.21 mmol), followed by 75% benzoyl peroxide (0.849 g, 2.51 mmol) and the reaction mixture heated to 70° C. overnight. Water and DCM were added to the reaction mixture, the organic layer separated, dried and concentrated in vacuo onto silica. Purification via column chromatography (0-15% EtOAc in petroleum ether) gave a yellow solid (0.725 g, 71%). ¹H NMR (400 MHz, CHLOROFORM-d) d 8.57 (s, 1H), 7.19-7.20 (m, 1H), 5.02 (s, 2H), 4.06 (s, 3H), 3.91 (s, 3H)

### Step 5: 5-methoxy-2-methyl-6-nitro-isoindolin-1-one

To methyl 2-(bromomethyl)-5-methoxy-4-nitro-benzoate (0.725 g, 2.5 mmol) in Et₃N (0.43 mL, 3.0 mmol) and MeOH (30 mL) was added methylamine (1.25 mL, 2.5 mmol) and the reaction mixture heated at reflux for 4 hours. The reaction mixture was cooled and concentrated in vacuo onto silica and purified via column chromatography to give a yellow solid (0.150 g, 27%). ¹H NMR (400 MHz, CHLOROFORM-d) d 8.25 (s, 1H), 7.14 (s, 1H), 4.43 (s, 2H), 4.03 (s, 3H), 3.20 (s, 3H); LCMS (m/z): 223.0 [M+H]⁺.

### Step 6: 6-amino-5-methoxy-2-methyl-isoindolin-1-one

A solution of 5-methoxy-2-methyl-6-nitro-isoindolin-1-one (0.030 g, 0.135 mmol) in IPA (2 mL) was passed through the H-Cube reactor fitted with a Pd/C cartridge, at 1 mL/min, 25° C., under ‘Full H₂’ twice. The solution was concentrated in vacuo to yield the product, 6-amino-5-methoxy-2-methyl-isoindolin-1-one (0.025 g, 0.130 mmol, 96% Yield); LCMS (m/z): 193.0 [M+H]⁺.

### Intermediate 17: Tert-butyl 7-amino-6-chloro-3,4-dihydro-1H-isoquinoline-2-carboxylate

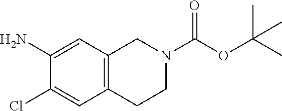 [SMILES: CC(C)(C)OC(=O)N1CCc2cc(Cl)c(N)cc2C1]

### Step 1: 6-Amino-7-nitro-3,4-dihydro-2H-isoquinolin-1-one

To a 20 mL microwave vial was added 6-fluoro-7-nitro-3,4-dihydro-2H-isoquinolin-1-one (0.5 g, 2.38 mmol) and ammonium hydroxide (10 mL). The vial was sealed and heated at 50° C. for 1 hour then concentrated in vacuo to give a bright yellow solid (0.525 g). ¹H NMR (400 MHz, DMSO-d₆) d ppm 8.43 (s, 1H), 7.87 (s, 1H), 7.73-7.81 (m, 2H), 6.79-6.83 (m, 1H), 3.31-3.34 (m, 2H), 2.79-2.85 (m, 2H); LCMS (m/z): 208.0 [M+H⁺].

### Step 2: 6-chloro-7-nitro-3,4-dihydro-2H-isoquinolin-1-one

To a suspension of 6-amino-7-nitro-3,4-dihydro-2H-isoquinolin-1-one (0.25 g, 1.20 mmol) in MeCN (20 mL) at 0° C. was added CuCl (0.179 g, 1.81 mmol) and ^{t}BuONO (0.21 mL, 1.81 mmol). The reaction mixture was left to stir and warm to room temperature over 2 hours. More CuCl (0.179 g, 1.81 mmol) and ^{t}BuONO (0.21 mL, 1.81 mmol) were added and the reaction mixture was heated at reflux for 1 hour. The reaction mixture was cooled. 2M HCl(aq) was added until no further effervescence was seen. The reaction mixture was poured into EtOAc, the organic layer was separated, washed with 2M aq. HCl, dried and concentrated in vacuo to give a yellow solid (0.226 g, 83%). ¹H NMR (400 MHz, DMSO-d₆) d ppm 8.37 (s, 1H), 8.34 (br. s., 1H), 7.83 (s, 1H), 3.39-3.45 (m, 1H), 2.99-3.04 (m, 1H).

### Step 3: 6-chloro-7-nitro-1,2,3,4-tetrahydroisoquinoline

To a solution of 6-chloro-7-nitro-3,4-dihydro-2H-isoquinolin-1-one (0.226 g, 1 mmol) in THF (10 mL) was added BH₃ (1M in THF, 3 mL, 3 mmol). The reaction mixture was refluxed overnight, then cooled. 2M aq. HCl (5 mL) was added. The reaction mixture was neutralized with saturated. aq. NaHCO₃, the organic phase was extracted with EtOAc, separated, dried and concentrated in vacuo. The crude was adsorbed onto silica and purified by column chromatography (20-100% EtOAc in PE, 0-20% MeOH in EtOAc) to give a yellow residue (0.054 g). ¹H NMR (400 MHz, CHLOROFORM-d) d ppm 7.63 (s, 1H), 7.30 (s, 1H), 4.06 (s, 2H), 3.18 (t, J=5.72 Hz, 2H), 2.88 (t, J=5.72 Hz, 2H); LCMS (m/z): 213 [M+H⁺].

### Step 4: tert-butyl 6-chloro-7-nitro-3,4-dihydro-1H-isoquinoline-2-carboxylate

To a solution of 6-chloro-7-nitro-1,2,3,4-tetrahydroisoquinoline (0.054 g, 0.25 mmol) in DCM (5 mL) was added Et₃N (74 ul, 0.51 mmol), BOC₂O (0.067 g, 0.030 mmol), and DMAP (3 mg, 0.025 mmol). The reaction mixture was left to stir at room temperature overnight, then diluted with DCM and water. The organic layer was separated, dried and concentrated in vacuo onto silica. Purification via column chromatography (5-20% EtOAC in PE) gave a yellow gum (0.05 g, 63% yield). ¹H NMR (400 MHz, CHLOROFORM-d) d ppm 7.70 (s, 1H), 7.34 (s, 1H), 4.60 (s, 2H), 3.64-3.70 (m, 2H), 2.86-2.91 (m, 2H), 1.50 (s, 9H); LCMS (m/z): 213 [M-CO₂^{t}Bu+H]⁺.

### Step 5: tert-butyl 7-amino-6-chloro-3,4-dihydro-1H-isoquinoline-2-carboxylate

A solution of tert-butyl 6-chloro-7-nitro-3,4-dihydro-1H-isoquinoline-2-carboxylate (0.050 g, 0.16 mmol) in EtOAc (10 mL) and MeOH (10 mL) was passed though the H-Cube reactor, fitted with a PtO₂ cartridge, at 1 mL/min at 25° C. under ‘Full H₂’ conditions, twice. The final solution was concentrated in vacuo to give the product (0.045 g). LCMS (m/z): 183/185 [M-CO₂^{t}Bu+H]⁺.

### Intermediate 18: Tert-butyl 4-(3-amino-4-chloro-phenoxy)piperidine-1-carboxylate

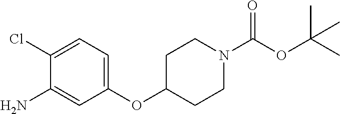 [SMILES: CC(C)(C)OC(=O)N1CCC(Oc2ccc(Cl)c(N)c2)CC1]

### Step 1: Tert-butyl 4-(4-chloro-3-nitro-phenoxy)piperidine-1-carboxylate

To a solution of 4-chloro-3-nitrophenol (0.500 g, 2.88 mmol) and K₂CO₃ (0.795 g, 5.76 mmol) in DMF was added tert-butyl 4-methylsulfonyloxypiperidine-1-carboxylate (0.881 g, 3.17 mmol) and the reaction mixture heated at 70° C. overnight. EtOAc and water were added to the reaction mixture, the organic layer extracted and washed with water. The organic layer was concentrated in vacuo onto silica and purified via column chromatography (5-15% EtOAc in PE). The fractions containing product were combined to give a yellow oil which was subsequently taken up in DCM and washed with 2M Na₂CO₃ (aq), the organic layer separated, dried and concentrated in vacuo to give the product as a yellow solid (0.649 g, 1.82 mmol, 63% Yield). ¹H NMR (CHLOROFORM-d) d: 7.44 (d, J=8.7 Hz, 1H), 7.40 (s, 1H), 7.05-7.09 (m, 1H), 4.43-4.60 (m, 1H), 3.63-3.73 (m, 2H), 3.27-3.45 (m, 2H), 1.89-2.02 (m, 2H), 1.76 (s, 2H), 1.48 (s, 9H). LCMS (m/z): 257.0 [M-CO₂^{t}Bu+H]⁺.

### Step 2: Tert-butyl 4-(3-amino-4-chloro-phenoxy)piperidine-1-carboxylate

To a solution of tert-butyl 4-(4-chloro-3-nitro-phenoxy)piperidine-1-carboxylate (0.649 g, 1.82 mmol) in EtOAc (30 mL) was added SnCl₂·2H₂O (2.06 g, 9.11 mmol) and the reaction mixture left to stir overnight. The reaction mixture was poured in saturated NaHCO₃ (aq), the organic layer separated, dried and concentrated in vacuo to give the product, tert-butyl 4-(3-amino-4-chloro-phenoxy)piperidine-1-carboxylate (0.330 g, 1.01 mmol, 56% Yield), as a white solid. ¹H NMR (CHLOROFORM-d) d: 7.12 (d, J=8.7 Hz, 1H), 6.34 (d, J=2.7 Hz, 1H), 6.28 (dd, J=8.7, 2.7 Hz, 1H), 4.33-4.42 (m, 1H), 4.11 (br. s, 2H), 3.61-3.73 (m, 2H), 3.27-3.38 (m, 2H), 1.84-1.94 (m, 2H), 1.65-1.78 (m, 2H), 1.47 (s, 9H). LCMS (m/z): 227.0 [M-CO₂^{t}Bu+H]⁺.

### Intermediate 19: 7-Amino-N,N-dimethyl-2,3-dihydrobenzofuran-5-sulfonamide

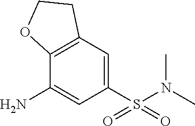 [SMILES: CN(C)S(=O)(=O)c1cc(N)c2c(c1)CCO2]

### Step 1: N,N-dimethyl-7-nitro-2,3-dihydrobenzofuran-5-sulfonamide

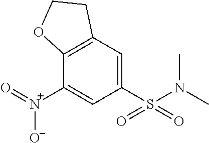 [SMILES: CN(C)S(=O)(=O)c1cc2c(c([N+](=O)[O-])c1)OCC2]

To a solution of 7-nitro-2,3-dihydrobenzofuran-5-sulfonyl chloride (0.100 g, 0.380 mmol) in DCM was added dimethylamine (0.021 g, 0.760 mmol) and the reaction mixture left to stir overnight at room temperature. The reaction mixture was concentrated in vacuo and purified via column chromatography (40-100% EtOAc in PE) to give a white solid (0.078 g, 76%). ¹H NMR (CHLOROFORM-d) 8.35-8.39 (m, 1H), 7.82 (d, J=1.4 Hz, 1H), 4.99 (t, J=8.9 Hz, 2H), 3.43 (t, J=8.9 Hz, 2H), 2.77 (s, 6H).

### Step 2: 7-Amino-N,N-dimethyl-2,3-dihydrobenzofuran-5-sulfonamide

A solution of N,N-dimethyl-7-nitro-2,3-dihydrobenzofuran-5-sulfonamide (0.078 g, 0.286 mmol) in EtOAc (10 mL) and MeOH (10 mL) was passed through the H-Cube reactor fitted with a 10% Pd/C cartridge continuous flow 1 mL/min, 25° C., under ‘Full H₂’. The solvent was removed to yield the product as a white solid (0.043 g, 62%). ¹H NMR (CHLOROFORM-d) d: 7.05-7.08 (m, 1H), 6.98 (d, J=1.8 Hz, 1H), 4.69 (t, J=8.9 Hz, 2H), 3.23-3.32 (m, 2H), 2.66-2.72 (m, 6H). LCMS (m/z): 243 [M+H]⁺.

### Intermediate 20: 5-Morpholinosulfonyl-2,3-dihydrobenzofuran-7-amine

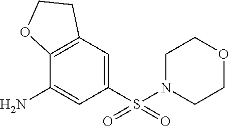 [SMILES: Nc1cc(S(=O)(=O)N2CCOCC2)cc2c1OCC2]

Prepared analogously to Intermediate 19 except morpholine was used in Step 1. ¹H NMR (CHLOROFORM-d) d: 6.99-7.09 (m, 1H), 6.95 (d, J=1.8 Hz, 1H), 4.70 (t, J=8.9 Hz, 2H), 3.69-3.77 (m, 4H), 3.37-3.64 (m, 2H), 3.27 (t, J=8.9 Hz, 2H), 2.90-3.06 (m, 4H); LCMS (m/z): 285 [M+H]⁺.

### Intermediate 21: Tert-butyl 4-[(7-amino-2,3-dihydrobenzofuran-5-yl)sulfonyl]piperazine-1-carboxylate

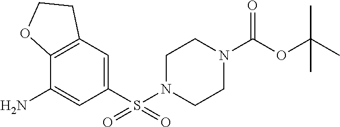 [SMILES: CC(C)(C)OC(=O)N1CCN(S(=O)(=O)c2cc(N)c3c(c2)CCO3)CC1]

Prepared analogously to Intermediate 19 except N-BOC-piperazine was used in step 1. ¹H NMR (CHLOROFORM-d) d: 6.99-7.07 (m, 1H), 6.94 (d, J=1.8 Hz, 1H), 4.70 (t, J=8.7 Hz, 2H), 3.48-3.56 (m, 4H), 3.27 (t, J=8.7 Hz, 2H), 2.91-3.01 (m, 4H), 1.42 (s, 9H)

### Intermediate 22: 3-Methoxy-1-(2-morpholinoethyl)pyrazol-4-amine

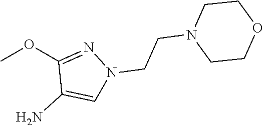 [SMILES: COc1nn(CCN2CCOCC2)cc1N]

### Step 1: 4-[2-(3-Methoxy-4-nitro-pyrazol-1-yl)ethyl]morpholine

To a mixture of 3-methoxy-4-nitro-1H-pyrazole (0.250 g, 1.75 mmol) and K₂CO₃ (0.483 g, 3.50 mmol) in DMF (5 mL) was added 4-(2-chloroethyl)morpholine hydrochloride (0.390 g, 2.10 mmol) and the reaction mixture heated at 70° C. overnight. After cooling, the mixture was concentrated onto silica gel and purified via column chromatography (70-100% EtOAc in PE, 0-20% MeOH in EtOAc) to give the product (0.280 g, 63%). ¹H NMR (CHLOROFORM-d) Shift: 8.12 (s, 1H), 3.97-4.17 (m, 5H), 3.65-3.79 (m, 4H), 2.79 (t, J=6.0 Hz, 2H), 2.24-2.55 (m, 4H). LCMS (m/z): 257.1 [M+H]⁺.

### Step 2: 3-Methoxy-1-(2-morpholinoethyl)pyrazol-4-amine

A solution of 4-[2-(3-methoxy-4-nitro-pyrazol-1-yl)ethyl]morpholine (0.140 g, 0.547 mmol) in EtOAc (30 mL) and MeOH (10 mL) was passed through the H-Cube reactor fitted with a 10% Pd/C cartridge, cf 1 mL/min, 25° C., under ‘Full H₂’, and subsequently concentrated in vacuo, yielding the product (0.104 g, 84%). ¹H NMR (CHLOROFORM-d) Shift: 6.95 (s, 1H), 3.96 (t, J=6.6 Hz, 2H), 3.90 (s, 3H), 3.66-3.71 (m, 4H), 2.70 (t, J=6.6 Hz, 2H), 2.47-2.62 (m, 2H), 2.42-2.47 (m, 4H). LCMS (m/z): 227.1 [M+H]⁺.

### Intermediate 23: 2-Chloro-5-(morpholinomethyl)aniline

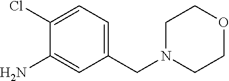 [SMILES: Nc1cc(CN2CCOCC2)ccc1Cl]

### Step 1: 4-Chloro-3-nitro-benzaldehyde

To a solution of (4-chloro-3-nitro-phenyl)methanol (0.500 g, 2.67 mmol) in THF (10 mL) was added Dess-Martin periodinane (0.752 g, 4.011 mmol) and the reaction mixture was stirred at room temperature for 2.5 h. The reaction mixture was concentrated in vacuo directly onto silica and purified via column chromatography (10-30% EtOAc in PE) to give a yellow solid (0.302 g, 61% yield). ¹H NMR (CHLOROFORM-d) d: 10.05 (s, 1H), 8.38 (d, J=1.8 Hz, 1H), 8.05 (dd, J=8.2, 1.8 Hz, 1H), 7.77 (d, J=8.2 Hz, 1H).

### Step 2: 4-[(4-Chloro-3-nitro-phenyl)methyl]morpholine

To a solution of 4-chloro-3-nitro-benzaldehyde (0.100 g, 0.541 mmol) in DCM was added morpholine (0.049 g, 0.568 mmol) and Na(OAc)₃BH (0.172 g, 0.811 mmol) and the reaction mixture was stirred at room temperature overnight. The mixture was concentrated in vacuo directly into silica gel and purified by column chromatography (30-100% EtOAc in PE) to give a yellow gum (0.132 g, 95%). ¹H NMR (CHLOROFORM-d) Shift: 7.85-7.90 (m, 1H), 7.49-7.52 (m, 2H), 3.68-3.76 (m, 4H), 3.53 (s, 2H), 2.38-2.53 (m, 4H). LCMS (m/z): 257.0 [M+H]⁺.

### Step 3: 2-Chloro-5-(morpholinomethyl)aniline

To a solution of 4-[(4-chloro-3-nitro-phenyl)methyl]morpholine (0.132 g, 0.516 mmol) in EtOAc was added SnCl₂·2H₂O (0.582 g, 2.58 mmol) and the reaction mixture left to stir overnight. Saturated NaHCO₃ (aq) was added to the reaction mixture, the organic layer separated, dried and concentrated in vacuo to give the desired product (0.110 g, 94% yield). ¹H NMR (CHLOROFORM-d) d: 7.18 (d, J=7.8 Hz, 1H), 6.82 (d, J=1.8 Hz, 1H), 6.59-6.73 (m, 1H), 4.05 (br. s., 2H), 3.67-3.80 (m, 4H), 3.42 (s, 2H), 2.36-2.54 (m, 4H). LCMS (m/z): 227.0 [M+H]⁺.

### Intermediate 24: 6-Chloro-1,2,3,4-tetrahydroisoquinolin-7-amine

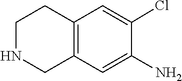 [SMILES: Nc1cc2c(cc1Cl)CCNC2]

### Step 1: Ethyl N-[2-(3-chlorophenyl)ethyl]carbamate

To a solution of 2-(3-chlorophenyl)ethanamine (5.1 g, 33.0 mmol) and Et₃N (7.12 mL, 49.4 mmol) in DCM (50 mL) was added ethylchloroformate (3.77 mL, 39.6 mmol) dropwise and the reaction mixture left to stir at room temperature for 3 hours. The reaction mixture was concentrated in vacuo and triturated with EtOAc. The mixture was filtered to remove a white solid, and the filtrate concentrated to give a yellow oil. The oil was concentrated onto silica and purified via column chromatography (15-100% EtOAc in PE), yielding the product (2.26 g, 9.95 mmol, 30% Yield). ¹H NMR (400 MHz, CHLOROFORM-d) d 7.16-7.25 (m, 3H), 7.04-7.09 (m, 1H), 4.58-4.76 (m, 1H), 4.10 (q, J=7.10 Hz, 2H), 3.41 (q, J=7.00 Hz, 2H), 2.78 (t, J=7.00 Hz, 2H), 1.22 (t, J=7.10 Hz, 3H).

### Step 2: 6-Chloro-3,4-dihydro-2H-isoquinolin-1-one

Ethyl N-[2-(3-chlorophenyl)ethyl]carbamate (2.26 g, 9.95 mmol) and polyphosphoric acid (15 mL) were heated at 120° C. for 5 hours. The reaction mixture was cooled to 70° C., poured into water and extracted with EtOAc. The organic layer was separated, washed with NaHCO₃ (aq), dried and concentrated in vacuo to give the product, 6-chloro-3,4-dihydro-2H-isoquinolin-1-one (0.632 g, 3.49 mmol, 35% Yield), as a white solid. ¹H NMR (400 MHz, CHLOROFORM-d) d 8.01 (d, J=8.24 Hz, 1H), 7.32-7.35 (m, 1H), 7.24 (d, J=2.29 Hz, 1H), 6.18-6.32 (m, 1H), 3.55-3.61 (m, 2H), 2.97-3.02 (m, 2H).

### Step 3: 6-Chloro-7-nitro-3,4-dihydro-2H-isoquinolin-1-one

To a solution 6-chloro-3,4-dihydro-2H-isoquinolin-1-one (0.434 g, 2.40 mmol) in H₂SO₄ (5 mL) at 0° C. was added HNO₃ (0.31 mL, 2.88 mmol) dropwise and the reaction mixture left to stir for 15 minutes. The reaction mixture was poured into water and extracted with EtOAc. The organic layer was separated, washed with NaHCO₃ (aq), dried and concentrated in vacuo to give a yellow solid. The solid was triturated with DCM/MeOH, the yellow solid collected and dried via vacuum filtration to give the product, 6-chloro-7-nitro-3,4-dihydro-2H-isoquinolin-1-one (0.354 g, 1.57 mmol, 65% Yield). ¹H NMR (400 MHz, DMSO-d6) d 8.28-8.38 (m, 2H), 7.81-7.85 (m, 1H), 3.39-3.45 (m, 2H), 2.98-3.05 (m, 2H).

### Step 4: 7-Amino-6-chloro-3,4-dihydro-2H-isoquinolin-1-one

To a solution of 6-chloro-7-nitro-3,4-dihydro-2H-isoquinolin-1-one (0.327 g, 1.45 mmol) in EtOAc (30 mL) was added SnCl₂·2H₂O (1.64 g, 7.23 mmol) and the reaction mixture left to stir overnight at room temperature. NaHCO₃ (aq) was added to the reaction mixture to give a white precipitate, which was subsequently removed via filtration. The organic layer of the filtrate was separated, dried and concentrated in vacuo to give the product, 7-amino-6-chloro-3,4-dihydro-2H-isoquinolin-1-one (0.230 g, 1.17 mmol, 81% Yield), as a light yellow solid. ¹H NMR (400 MHz, CHLOROFORM-d) d 7.48 (s, 1H), 7.13 (s, 1H), 6.00-6.11 (m, 1H), 3.50-3.56 (m, 2H), 2.86-2.91 (m, 2H).

### Step 5: 6-Chloro-1,2,3,4-tetrahydroisoquinolin-7-amine

To 7-amino-6-chloro-3,4-dihydro-2H-isoquinolin-1-one (0.050 g, 0.255 mmol) in THF (3 mL) was added LiAlH₄ (2M in THF) (0.65 mL, 1.78 mmol) dropwise, and the reaction heated at reflux for 30 minutes. Incomplete conversion was noted via NMR, hence a further equivalent of LiAlH₄ was added, and the reaction mixture stirred at reflux for 4.5 hours. The reaction mixture was cooled, H₂O, 15% NaOH+H₂O added, the mixture filtered and passed through a phase separator. The eluent was concentrated in vacuo to give the product, 6-chloro-1,2,3,4-tetrahydroisoquinolin-7-amine (0.046 g, 0.247 mmol, 94% Yield), as a cream solid. ¹H NMR (400 MHz, CHLOROFORM-d) d 6.98 (s, 1H), 6.43 (s, 1H), 3.87-3.96 (m, 4H), 3.04-3.09 (m, 2H), 2.64-2.69 (m, 2H).

### Intermediate 25: 6-Amino-5-methoxy-2-methyl-isoindolin-1-one

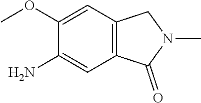

### Step 1: Methyl 4-fluoro-2-methyl-benzoate

To a solution of 4-fluoro-2-methyl benzoic acid (2.5 g, 16.2 mmol) in MeOH (100 mL) was added H₂SO₄ (5 mL) and the reaction mixture heated at 70° C. overnight. The reaction mixture was cooled, diluted with DCM and H₂O, the organic layer separated, washed with NaHCO₃ (aq.), dried and concentrated in vacuo to give the product, methyl 4-fluoro-2-methyl-benzoate (1.0 g, 6.49 mmol, 37% Yield), as a brown oil. ¹H NMR (400 MHz, CHLOROFORM-d) d 7.96 (dd, J=6.18, 8.47 Hz, 1H), 6.89-6.97 (m, 2H), 3.89 (s, 3H), 2.61 (s, 3H).

### Step 2: Methyl 4-fluoro-2-methyl-5-nitro-benzoate

To methyl 4-fluoro-2-methyl-benzoate (1.0 g, 6.49 mmol) in H₂SO₄ (10 mL), at 5° C., was added HNO₃ (0.30 mL, 7.14 mmoL) dropwise and left to stir for 45 minutes. The reaction mixture was poured into H₂O and extracted with EtOAc. The organic layer was separated, dried and concentrated in vacuo to give a yellow oil that solidified upon standing as the product, methyl 4-fluoro-2-methyl-5-nitro-benzoate (1.22 g, 6.12 mmol, 95% Yield). ¹H NMR (400 MHz, CHLOROFORM-d) d 8.71 (d, J=8.00 Hz, 1H), 7.20 (d, J=11.70 Hz, 1H), 3.95 (s, 3H), 2.71 (s, 3H).

### Step 3: Methyl 4-methoxy-2-methyl-5-nitro-benzoate

To methyl 4-fluoro-2-methyl-5-nitro-benzoate (1.22 g, 6.12 mmol) in MeOH (50 mL) was added NaOMe (0.347 g, 6.43 mmol) and the reaction mixture heated at reflux for 4 hours. The reaction mixture was cooled, H₂O and EtOAc added, the organic layer separated, dried and concentrated in vacuo onto silica. The compound was purified via column chromatography (5-10% EtOAc in PE), and the fractions containing product were combined and concentrated in vacuo to yield the product, methyl 4-methoxy-2-methyl-5-nitro-benzoate (0.741 g, 3.51 mmol, 57% Yield), as a white solid. ¹H NMR (400 MHz, CHLOROFORM-d) d 8.58 (s, 1H), 6.93 (s, 1H), 4.02 (s, 3H), 3.91 (s, 3H), 2.72 (s, 3H).

### Step 4: Methyl 2-(bromomethyl)-4-methoxy-5-nitro-benzoate

To methyl 4-methoxy-2-methyl-5-nitro-benzoate (0.741 g, 3.51 mmol) in MeCN (20 mL) was added NBS (0.750 g, 4.21 mmol), followed by benzoyl peroxide (75%) (0.849 g, 2.51 mmol) and the reaction mixture heated to 70° C. overnight. H₂O and DCM were added to the reaction mixture, the organic layer separated, dried and concentrated in vacuo onto silica. Purification was attempted via column chromatography (0-15% EtOAc in PE), yielding the product, methyl 2-(bromomethyl)-4-methoxy-5-nitro-benzoate (0.725 g, 2.51 mmol, 71% Yield), as a yellow solid. ¹H NMR (400 MHz, CHLOROFORM-d) d 8.57 (s, 1H), 7.19-7.20 (m, 1H), 5.02 (s, 2H), 4.06 (s, 3H), 3.91 (s, 3H).

### Step 5: 5-Methoxy-2-methyl-6-nitro-isoindolin-1-one

To methyl 2-(bromomethyl)-5-methoxy-4-nitro-benzoate (0.725 g, 2.5 mmol) in Et₃N (0.43 mL, 3.0 mmol) and MeOH (30 mL) was added methylamine (1.25 mL, 2.5 mmol) and the reaction mixture heated at reflux for 4 hours. The reaction mixture was cooled and concentrated in vacuo onto silica and purified via column chromatography to give the product, 5-methoxy-2-methyl-6-nitro-isoindolin-1-one (0.150 g, 0.676 mmol, 27% Yield), as a yellow solid. ¹H NMR (400 MHz, CHLOROFORM-d) d 8.25 (s, 1H), 7.14 (s, 1H), 4.43 (s, 2H), 4.03 (s, 3H), 3.20 (s, 3H); LCMS (m/z): 223.0 [M+H]⁺.

### Step 6: 6-Amino-5-methoxy-2-methyl-isoindolin-1-one

A solution of 5-methoxy-2-methyl-6-nitro-isoindolin-1-one (0.030 g, 0.135 mmol) in IPA (2 mL) was passed through the H-Cube reactor fitted with a Pd/C cartridge, at 1 mL/min, 25° C., under ‘Full H₂’ twice. The solution was concentrated in vacuo to yield the product, 6-amino-5-methoxy-2-methyl-isoindolin-1-one (0.025 g, 0.130 mmol, 96% Yield). LCMS (m/z) 193.0 [M+H]⁺.

Intermediate 26: 2-Chloro-5-(trifluoromethyl)-N-[(1-tritylpyrazol-3-yl)methyl]pyrimidin-4-amine

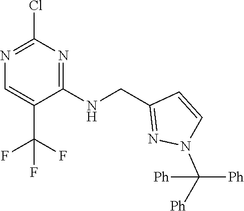 [SMILES: FC(F)(F)c1cnc(Cl)nc1NCc1ccn(C(c2ccccc2)(c2ccccc2)c2ccccc2)n1]

### Step 1: (3E)-1-tritylpyrazole-3-carbaldehyde oxime

1-tritylpyrazole-3-carbaldehyde (9.2 g, 2.72 mmol), hydroxylamine hydrochloride (3.8 g, 5.44 mmol), sodium acetate (4.5 g, 5.44 mmol) were combined in methanol (100 mL) and water (10 mL) and the reaction mixture heated at reflux for 1.5 h. The reaction mixture was cooled down and concentrated in vacuo, DCM added, the organic phase separated, dried and concentrated in vacuo to give the product (3E)-1-tritylpyrazole-3-carbaldehyde oxime (9.4 g, 98%) as a white solid. ¹H NMR (400 MHz, DMSO-d6) d 11.18 (s, 1H), 11.12-11.31 (m, 1H), 7.93-8.09 (m, 1H), 7.34-7.39 (m, 9H), 7.31-7.44 (m, 10H), 7.02-7.12 (m, 6H), 6.45-6.60 (m, 1H).

### Step 2: (1-tritylpyrazol-3-yl)methanamine

To a solution of the (3E)-1-tritylpyrazole-3-carbaldehyde oxime (9.37 g, 27 mmol) in THF (100 mL) at 0° C. was added lithium aluminium hydride in THF (53.1 mL, 53 mmol) dropwise. The reaction mixture was heated at 65° C. for 1.5 h. The reaction mixture was cooled, ice added and the reaction mixture quenched by a careful addition of methanol. The solution/gel that formed was filtered and washed with methanol. The filtrate was concentrated in vacuo onto silica, the silica split between two 50 g SNAP cartridges and the compound purified via column chromatography (5-10% MeOH in DCM). The fractions containing the product were combined and concentrated in vacuo to give (1-tritylpyrazol-3-yl)methanamine as yellow solid (4.55 g, 55%). ¹H NMR (400 MHz, DMSO-d6) 7.35-7.47 (m, 10H), 7.03-7.16 (m, 6H), 6.26-6.39 (m, 1H), 3.66-3.73 (m, 2H), 1.69 (t, J=7.10 Hz, 2H).

### Step 3: 2-Chloro-5-(trifluoromethyl)-N-[(1-tritylpyrazol-3-yl)methyl]pyrimidin-4-amine

To a suspension of (1-tritylpyrazol-3-yl)methanamine (3.3 g, 9.65 mmol) and triethylamine (2.8 mL, 1.93 mmol) in IPA (80 mL) was added 2,4-dichloro-5-trifluoromethyl pyrimidine (2.1 g, 9.65 mmol) and stirred at 50° C. overnight. The reaction mixture was cooled down, diluted with DCM and water, the organic layer separated, dried and concentrated in vacuo onto silica. The compound was purified via column chromatography (5-20% ethyl acetate in petroleum ether). The fractions containing the first peak were combined in vacuo to give 2-chloro-5-(trifluoromethyl)-N-[(1-tritylpyrazol-3-yl)methyl]pyrimidin-4-amine (1.57 g, 32%) as a white solid. ¹H NMR (400 MHz, CHLOROFORM-d) d 8.19-8.29 (m, 1H), 7.31-7.34 (m, 8H), 7.05-7.17 (m, 7H), 6.35 (br. s., 1H), 6.20 (d, J=2.29 Hz, 1H), 4.67-4.75 (m, 2H) HPLC Rt=2.41 min (MeCN, pH10).

### Example 1: 1-(3-(2-(7-Chloro-1, 2, 3, 4-tetrahydroisoquinolin-6-ylamino)-5(trifluoromethyl) pyrimidin-4-ylamino) propyl) piperidin-2-one

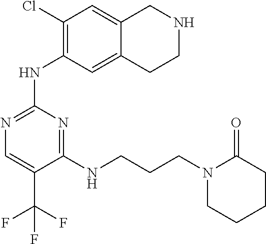 [SMILES: O=C1CCCCN1CCCNc1nc(Nc2cc3c(cc2Cl)CNCC3)ncc1C(F)(F)F]

### Step 1: Synthesis of 1-(3-(2-(7-Chloro-2-(2, 2, 2-trifluoroacetyl)-1, 2, 3, 4-tetrahydroisoquinolin-6-ylamino)-5-(trifluoromethyl) pyrimidin-4-ylamino) propyl) piperidin-2-one

To a solution of Intermediate 1 (10 g, 29.8 mmol) in IPA (100 mL) was added Intermediate 2 (9.9 g, 35.7 mmol) and the reaction mixture was heated to 80° C. for 16 h. The reaction mixture was concentrated, and the crude compound was purified by column chromatography (SiO₂, 100-200 mesh, 50% ethyl acetate in pet ether) to afford a light brown solid (9.5 g, 55%) which was used in the next step without purification.

### Step 2: 1-(3-(2-(7-Chloro-1, 2, 3, 4-tetrahydroisoquinolin-6-ylamino)-5(trifluoromethyl) pyrimidin-4-ylamino) propyl) piperidin-2-one

To a solution of 1-(3-(2-(7-chloro-2-(2,2,2-trifluoroacetyl)-1,2,3,4-tetrahydroisoquinolin-6-ylamino)-5-(trifluoromethyl)pyrimidin-4-ylamino)propyl)piperidin-2-one (10 g, 17.2 mmol) in a mixture of ethanol (120 mL) and water (60 mL) was added potassium carbonate (9.5 g, 68.8 mmol) at room temperature. The mixture was heated to 70° C. for 3 h, cooled to room temperature and concentrated. Purification by column chromatography (SiO2, 6% MeOH in DCM) followed by preparative HPLC gave the title compound as an off white solid (3.5 g, 42%). ¹H NMR (400 MHz, CDCl₃) δ 8.22 (s, 1H), 8.17 (s, 1H), 7.37 (s, 1H), 7.05 (s, 1H), 6.80 (s, 1H), 3.97 (s, 2H), 3.44-3.51 (m, 4H), 3.28 (d, J=5.8 Hz, 2H), 3.16 (t, J=6.0 Hz, 2H), 2.81 (t, J=5.8 Hz, 2H), 2.44 (t, J=5.8 Hz, 2H), 1.83-1.78 (m, J=4.9 Hz, 6H); LCMS (m/z)=483.56 [M+H]⁺

### Example 2: (S)-3-(2-(7-chloro-1,2,3,4-tetrahydroisoquinolin-6-ylamino)-5-(trifluoromethyl)pyrimidin-4-ylamino)-N-(1-cyclopropyl-2,2,2-trifluoroethyl)propanamide

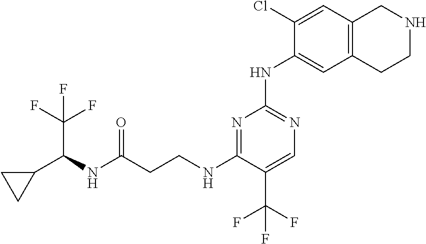 [SMILES: O=C(CCNc1nc(Nc2cc3c(cc2Cl)CNCC3)ncc1C(F)(F)F)N[C@@H](C1CC1)C(F)(F)F]

### Step 1: Synthesis of ethyl 3-(2-chloro-5-(trifluoromethyl) pyrimidin-4-ylamino) propanoate

To a solution of ethyl 3-aminopropanoate hydrochloride (500 mg, 3.25 mmol) in IPA (5 mL) was added N,N-diisopropylethylamine (0.93 g, 7.16 mmol) at 0° C. and the reaction mixture was stirred at the same temperature for 30 min. The reaction mixture was cooled to −78° C. and a solution of 2,4-dichloro-5-(trifluoromethyl)pyrimidine (0.85 g, 3.91 mmol) in IPA (5 mL) was added and the reaction mixture was warmed to room temperature and stirred for 2 h. The reaction mixture was concentrated, diluted with ethyl acetate and washed with water. The organic phase was dried over anhydrous sodium sulfate, filtered and concentrated. The crude product was purified by reverse phase column chromatography [C18, 0.1% aq. HCOOH in MeCN] to afford ethyl 3-(2-chloro-5-(trifluoromethyl) pyrimidin-4-ylamino) propanoate (400 mg, 41%). ¹H NMR (400 MHz, CDCl₃): δ ppm 8.27 (d, J=0.8 Hz, 1H), 6.31 (brs, 1H), 4.21 (q, J=14.4 Hz & 7.2 Hz, 2H), 3.88 (q, J=11.6 Hz & 6.0 Hz, 2H), 2.68 (t, J=6.0 Hz, 2H), 1.29 (t, J=7.2 Hz, 3H). LCMS (m/z): 298.0 [M+H]⁺

### Step 2: Synthesis of 3-(2-chloro-5-(trifluoromethyl) pyrimidin-4-ylamino) propanoic acid

To a solution of ethyl 3-(2-chloro-5-(trifluoromethyl) pyrimidin-4-ylamino) propanoate (0.35 g, 1.17 mmol) in THF:water (2:1, 10 mL) was added lithium hydroxide monohydrate (0.15 g, 3.57 mmol) at room temperature and stirred for 1 h. The reaction mixture was quenched with saturated aqueous citric acid solution and extracted with ethyl acetate. The organic layer was dried over anhydrous sodium sulfate, filtered and concentrated to afford crude 3-(2-chloro-5-(trifluoromethyl) pyrimidin-4-ylamino) propanoic acid (300 mg which was in the next step without purification. LCMS (m/z): 270.0 [M+H]⁺

### Step 3: Synthesis of (S)-3-(2-chloro-5-(trifluoromethyl) pyrimidin-4-ylamino)-N-(1-cyclopropyl-2,2,2-trifluoroethyl)propanamide

To a solution of 3-(2-chloro-5-(trifluoromethyl)pyrimidin-4-ylamino)propanoic acid (300 mg, crude) in THF (5 mL) was added N,N-diisopropylethylamine (360 mg, 2.78 mmol), (S)-1-cyclopropyl-2,2,2-trifluoroethanamine hydrochloride (180 mg, 1.02 mmol) and 1-propanephosphonic acid anhydride (50% in ethyl acetate, 1.5 mL, 2.33 mmol) at room temperature and the reaction mixture was stirred for 3 h. The reaction mixture was diluted with ethyl acetate, washed with water and brine and the organic layer was dried over anhydrous sodium sulfate, filtered and concentrated to afford (S)-3-(2-chloro-5-(trifluoromethyl) pyrimidin-4-ylamino)-N-(1-cyclopropyl-2,2,2-trifluoroethyl)propa-namide (450 mg), which was used directly used in the next step without purification LCMS (m/z): 391.0 [M+H]⁺.

### Step 4: Synthesis of (S)-3-(2-(7-chloro-2-(2,2,2-trifluoroacetyl)-1,2,3,4-tetrahydroisoquinolin-6-ylamino)-5-(trifluoromethyl)pyrimidin-4-ylamino)-N-(1-cyclopropyl-2,2,2-trifluoroethyl)-propanamide

To a solution of (S)-3-(2-chloro-5-(trifluoromethyl)pyrimidin-4-ylamino)-N-(1-cyclopropyl-2,2,2-trifluoroethyl)propanamide from step 3 (450 mg) in IPA (10 mL) was added Intermediate 2 (380 mg, 1.38 mmol) at room temperature and the reaction mixture was heated to 80° C. for 16 h. The reaction mixture was concentrated under reduced pressure and the crude product was triturated with Et₂O to afford an off-white solid (350 mg). The crude product was used directly used in the next step. LCMS (m/z): 633.0 [M+H]⁺

### Step 5: Synthesis of (S)-3-(2-(7-chloro-1,2,3,4-tetrahydroisoquinolin-6-ylamino)-5-(trifluoromethyl)pyrimidin-4-ylamino)-N-(1-cyclopropyl-2,2,2-trifluoroethyl)propanamide

To a solution of (S)-3-(2-(7-chloro-2-(2,2,2-trifluoroacetyl)-1,2,3,4-tetrahydroisoquinolin-6-ylamino)-5-(trifluoromethyl)pyrimidin-4-ylamino)-N-(1-cyclopropyl-2,2,2-trifluoroethyl)propanamide (350 mg, crude) in ethanol:water (2:1, 7.5 mL) was added K₂CO₃ (230 mg, 1.66 mmol) and the reaction mixture was stirred at room temperature for 2 h. The reaction mixture was concentrated under reduced pressure, the crude product was suspended in MeCN and methanol, filtered through Celite pad and the filtrate was concentrated. Purification by prep HPLC afforded the title compound as an off-white solid (90 mg, 14% over steps 2-4). ¹H NMR (400 MHz, DMSO-d₆): δ ppm 8.68 (s, 1H), 8.50 (d, J=9.2 Hz, 1H), 8.13 (s, 1H), 7.52 (s, 1H), 7.12 (s, 1H), 6.88 (t, J=5.2 Hz, 1H), 4.01-3.99 (m, 1H), 3.78 (s, 2H), 3.58 (q, J=6.0 Hz, 2H), 2.89 (t, J=5.6 Hz, 2H), 2.62 (t, J=5.6 Hz, 2H), 2.51-2.49 (m, 2H), 1.04-1.00 (m, 1H), 0.62-0.57 (m, 1H), 0.47-0.43 (m, 2H), 0.22-0.26 (m, 1H). LCMS (m/z): 537.0 [M+H]⁺

### Example 3: 1-(3-((2-((1-(2-(Dimethyl amino) ethyl)-3-methoxy-1H-pyrazol-4-yl) amino)-5-(trifluoromethyl) pyrimidin-4-yl) amino) propyl) piperidin-2-one

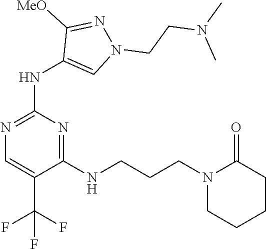 [SMILES: COc1nn(CCN(C)C)cc1Nc1ncc(C(F)(F)F)c(NCCCN2CCCCC2=O)n1]

### Step 1: Synthesis of 2-(3-methoxy-4-nitro-1H-pyrazol-1-yl)-N,N-dimethylethan-1-amine

To a stirred solution of 3-methoxy-4-nitro-1H-pyrazole (0.5 g, 3.49 mmol) in DMF (5 mL) at RT was added K₂CO₃ (1.44 g, 10.5 mmol) at room temperature followed by 2-chloro-N,N-dimethylethan-1-amine·HCl (0.6 g, 4.19 mmol) and the reaction mixture was heated to 70° C. for 16 h. The reaction mixture was diluted with water and extracted with ethyl acetate and the organic phase was washed with brine, dried over anhydrous sodium sulfate, filtered and concentrated. Purification by column chromatography using silica 100-200 gel (eluent: 10% MeOH:DCM) gave a light yellow solid (0.55 g, 73%). ¹H NMR (400 MHz, CDCl₃): δ 8.12 (s, 1H) 4.05-4.02 (m, 5H), 2.72 (t, J=6 Hz, 2H), 2.27 (s, 6H); LCMS (m/z): 215.30 [M+H]⁺

### Step 2: Synthesis of 1-(2-(dimethyl amino) ethyl)-3-methoxy-1H-pyrazol-4-amine

To a stirred solution of 2-(3-methoxy-4-nitro-1H-pyrazol-1-yl)-N,N-dimethylethan-1-amine (0.55 g, 2.57 mmol) in MeOH (15 mL) was added 10% Pd/C (0.5 g) at RT and stirred for 4 h under H₂ pressure (balloon pressure). The reaction mixture was filtered through Celite and washed with MeOH. Concentration of the filtrate afforded crude 1-(2-(dimethyl amino) ethyl)-3-methoxy-1H-pyrazol-4-amine (0.47 g, quantitative) which was used in the next step. ¹H NMR (400 MHz, CDCl₃): δ 6.95 (s, 1H) 3.95 (t, J=6.4 Hz, 2H) 3.91 (s, 3H), 2.65 (t, J=6.8 Hz, 2H), 2.27 (s, 6H); LCMS (m/z): 185.27 [M+H]⁺

### Step 3: 1-(3-((2-((1-(2-(dimethyl amino) ethyl)-3-methoxy-1H-pyrazol-4-yl) amino)-5-(trifluoromethyl) pyrimidin-4-yl) amino) propyl) piperidin-2-one

To a solution of 1-(2-(dimethyl amino) ethyl)-3-methoxy-1H-pyrazol-4-amine (0.45 g, 2.44 mmol) in IPA (20 mL) was added Intermediate 1 (0.66 g, 1.96 mmol) and the mixture was heated at 80° C. in a sealed tube for 12 h. The reaction mixture was concentrated and purified by preparative HPLC to give the title compound as an off-white solid (0.098 g, 10%). ¹H NMR (400 MHz, DMSO-d₆): δ 8.48 (brs, 1H), 8.05 (s, 1H), 7.68 (brs, 1H), 7.03 (brs, 1H), 3.99 (brs, 2H), 3.77 (s, 3H), 3.27-3.20 (m, 6H), 2.58 (t, J=6.4 Hz, 2H), 2.21-2.16 (m, 8H), 1.69 (brs, 6H); LCMS (m/z): 485.28 [M+H]⁺

### Example 4: 1-(3-((2-((7-Methoxy-1,2,3,4-tetrahydroisoquinolin-6-yl)amino)-5-(trifluoromethyl)pyrimidin-4-yl)amino)propyl)piperidin-2-one

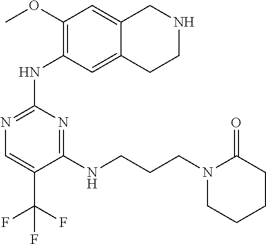 [SMILES: COc1cc2c(cc1Nc1ncc(C(F)(F)F)c(NCCCN3CCCCC3=O)n1)CCNC2]

### Step 1: Synthesis of 1-(3-((2-((7-methoxy-2-(2,2,2-trifluoroacetyl)-1,2,3,4-tetrahydroisoquinolin-6-yl)amino)-5-(trifluoromethyl)pyrimidin-4-yl)amino)propyl)piperidin-2-one

To a solution of Intermediate 3 (0.30 g, 1.09, mmol) in IPA (6.0 mL) was added Intermediate 1 (0.366 g, 1.09 mmol) at room temperature and heated to 80° C. for 16 h. After concentration under reduced pressure, the crude compound was purified by column chromatography (SiO₂, 100-200 mesh, 6% methanol in dichloromethane) to obtain and off-white solid (0.30 g, 48%). ¹H NMR (400 MHz, DMSO-d₆): δ 8.19 (s, 2H), 7.93 (s, 1H), 7.25 (brs, 1H), 6.97 (brs, 1H), 4.72 (s, 2H), 3.85 (s, 3H), 3.81 (t, J=6 Hz, 2H) 3.43 (q, J=12.4 & 10.4 Hz, 2H), 3.31 (t, J=6.8 Hz, 2H), 3.21 (m, 2H), 2.87 (s, 2H), 2.21 (t, J=6.4 Hz, 2H), 1.80-1.69 (m, 6H); LCMS (m/z): 575.6 [M+H]⁺

### Step 2: Synthesis of 1-(3-((2-((7-methoxy-1,2,3,4-tetrahydroisoquinolin-6-yl)amino)-5-(trifluoromethyl)pyrimidin-4-yl)amino)propyl)piperidin-2-one

To a solution of 1-(3-((2-((7-methoxy-2-(2,2,2-trifluoroacetyl)-1,2,3,4-tetrahydroisoquinolin-6-yl)amino)-5-(trifluoromethyl)pyrimidin-4-yl)amino)propyl)piperidin-2-one (0.30 g, 0.52 mmol) in a mixture of ethanol (12 mL) and water (6 mL) was added potassium carbonate (0.36 g, 2.60 mmol), and the mixture was stirred for 3 h at 70° C. The mixture was concentrated under reduced pressure and partitioned between water and ethyl acetate. The aqueous phase was extracted with ethyl acetate, the combined organic layers was dried (Na₂SO₄) and concentrated. Purification by preparative HPLC gave an off-white solid (0.054 g, 21%). ¹H NMR (400 MHz, DMSO-d₆): δ 8.14 (s, 1H), 7.93 (s, 1H), 7.80 (s, 1H), 7.23 (brs, 1H), 6.67 (s, 1H), 3.79 (s, 5H), 3.38-3.32 (m, 4H), 3.21 (s, 2H), 2.91-2.90 (m, 2H), 2.60 (s, 2H), 2.24-2.22 (m, 2H), 1.72-1.71 (m, 6H); LCMS (m/z): 479.3 [M+H]⁺

### Example 5: N2-(3-fluorophenyl)-N4-(3-(pyrrolidin-1-yl)propyl)-5-(trifluoromethyl)pyrimidine-2,4-diamine

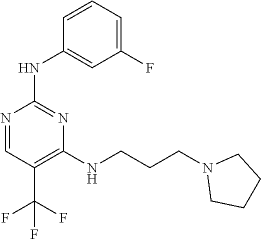 [SMILES: Fc1cccc(Nc2ncc(C(F)(F)F)c(NCCCN3CCCC3)n2)c1]

To a solution of Intermediate 6 (120 mg, 0.38 mmol) in tert-butanol (6 mL) was added trifluoracetic acid (174 mg, 1.52 mmol) and 3-fluoroaniline (34 mg, 0.30 mmol) at room temperature and the mixture was stirred at 100° C. for 3 h. The reaction mixture was evaporated, the residue was basified with 1N NaOH solution, extracted with ethyl acetate (2×20 mL). Combined organic layers were washed with brine, dried over anhydrous sodium sulfate, filtered and concentrated under reduced pressure. The crude solid was purified by triturating with diethyl ether to afford an off-white solid (60 mg, 40%). ¹H NMR (400 MHz, DMSO-d₆): δ 9.81 (s, 1H), 8.20 (s, 1H), 7.85-7.79 (m, 2H), 7.46 (dd, J=0.8 Hz, J₂=9.2 Hz, 1H), 7.31-7.25 (m, 1H), 6.76 (td, J=2.4, J₂=8.4 Hz, 1H), 3.51 (q, J=6.4 Hz, 2H), 2.54 (t, J=6.4 Hz, 2H), 2.43 (brs, 4H), 1.80-1.74 (m, 2H), 1.68 (brs, 4H); LCMS (m/z): 384.47 (M+H)⁺

### Example 6: N2-(2-methyl-1,2,3,4-tetrahydroisoquinolin-6-yl)-N4-(3-(pyrrolidin-1-yl)propyl)-5-(trifluoromethyl)pyrimidine-2,4-diamine

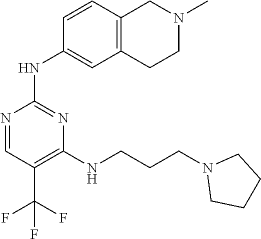 [SMILES: CN1CCc2cc(Nc3ncc(C(F)(F)F)c(NCCCN4CCCC4)n3)ccc2C1]

To a solution of Intermediate 6 (120 mg, 0.38 mmol) in tert-butanol (6 mL) was added trifluoracetic acid (174 mg, 2.2 mmol) and Intermediate 4 (50 mg, 0.30 mmol) and the mixture was stirred at 100° C. for 3 h. The reaction mixture was evaporated, the residue was basified with 1N NaOH solution, extracted with ethyl acetate (2×20 mL). The combined organic layers were washed with brine, dried over anhydrous sodium sulfate, filtered and concentrated under reduced pressure. The crude solid was purified by triturating with diethyl ether to afford an off-white solid (70 mg, 41.4%). ¹H NMR (400 MHz, DMSO-d₆): δ 9.45 (br s, 1H), 8.14 (s, 1H), 7.63 (t, J=4.4 Hz, 1H) 7.55 (s, 1H), 7.43 (dd, J=2.0, J₂=8.4 Hz, 1H), 6.92 (d, J=8.4 Hz, 1H), 3.51 (t, J=6.4 Hz, 2H), 3.40 (s, 2H), 2.77 (t, J=6.0 Hz, 2H), 2.57-2.53 (m, 4H), 2.42 (brs, 4H), 2.31 (s, 3H), 1.79-1.72 (m, 2H), 1.67 (brs, 4H); LCMS (m/z): 435.52 (M+H)⁺

### Example 7: 1-(3-((2-((2-Methyl-1,2,3,4-tetrahydroisoquinolin-6-yl)amino)-5-(trifluoromethyl) pyrimidin-4-yl)amino)propyl)piperidin-2-one

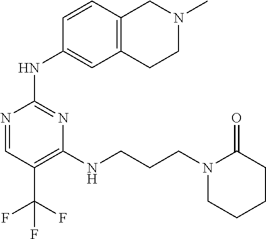 [SMILES: CN1CCc2cc(Nc3ncc(C(F)(F)F)c(NCCCN4CCCCC4=O)n3)ccc2C1]

To a solution of Intermediate 1 (170 mg, 0.50 mmol) in n-butanol (8 mL) was added trifluoracetic acid (228 mg, 2.0 mmol) and Intermediate 6 (65 mg, 0.40 mmol) and the mixture was stirred at 100° C. for 5 h. The reaction mixture was evaporated, the residue was basified with 1N NaOH solution and extracted with ethyl acetate (2×20 mL). The combined organic layers were washed with brine, dried over anhydrous sodium sulfate, filtered and concentrated under reduced pressure. The crude solid was purified by triturating with diethyl ether to afford and off-white solid (53 mg, 22%). ¹H NMR (400 MHz, DMSO-d₆): δ 9.48 (br s, 1H), 8.15 (s, 1H), 7.55 (s, 1H), 7.40 (d, J=8.0 Hz, 1H), 7.19 (brs, 1H), 6.94 (d, J=8.0 Hz, 1H), 3.40 (brs, 4H), 3.31 (brs, 2H), 3.14-3.18 (m, 2H), 2.82-2.76 (m, 2H), 2.56 (t, J=4.8 Hz, 2H), 2.32 (s, 3H), 2.24-2.18 (m, 2H), 1.76-1.68 (m, 6H); LCMS (m/z): 463.5 (M+H)⁺

### Example 8: 1-[3-[[2-[3-[(1-Methyl-4-piperidyl)oxy]anilino]-5-(trifluoromethyl)pyrimidin-4-yl]amino]propyl]piperidin-2-one

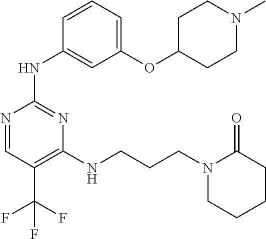 [SMILES: CN1CCC(Oc2cccc(Nc3ncc(C(F)(F)F)c(NCCCN4CCCCC4=O)n3)c2)CC1]

### Step 1: Synthesis of 1-methyl-4-(3-nitrophenoxy) piperidine

To a solution of 1-fluoro-3-nitrobenzene (5 g, 35.4 mmol) in dimethyl sulfoxide (25 mL) was added sodium tert-butoxide (3.74 g, 39.0 mmol) and the mixture was stirred for 10 min at 10° C. prior to addition of 1-methylpiperidin-4-ol (6.1 g, 53.1 mmol) and the mixture was allowed to stir at room temperature for 3 h. The reaction mixture was poured in ice water and extracted with ethyl acetate. The organic layer was washed with brine, dried over anhydrous sodium sulfate, filtered and concentrated to afford a yellow oil (4.0 g, 48%) which was used in Step 2 without further purification.

### Step 2: Synthesis of 3-((1-methylpiperidin-4-yl)oxy)aniline

A Parr shaker vessel was charged with a solution of 1-methyl-4-(3-nitrophenoxy) piperidine (4 g, 16.9 mmol) in methanol (50 mL) and 10% Pd/C (0.40 g) was added under nitrogen atmosphere. The mixture was hydrogenated at 30 psi for 5 h. The reaction mixture was filtered through a Celite and washed with methanol. The filtrate was concentrated to afford a yellow solid (3.0 g, 86%) which was used in the next step without purification.

### Step 3: Synthesis of 1-(3-((2-((3-((1-methylpiperidin-4-yl)oxy)phenyl)amino)-5-(trifluoromethyl)-pyrimidin-4-yl)amino)propyl)piperidin-2-one

To a solution of Intermediate 1 (100 mg, 0.297 mmol) in t-butanol (5 mL) was added TFA (0.9 mL, 1.19 mmol) and 3-((1-methylpiperidin-4-yl)oxy)aniline (50 mg, 0.238 mmol) and the mixture was stirred at 100° C. for 4 h. The mixture was concentrated under reduced pressure and the residue was suspended in water (20 mL), basified with aq. 1N NaOH and extracted with ethyl acetate (2×30 mL). The combined organic layers were washed with brine (30 mL), dried over anhydrous Na₂SO₄, filtered and concentrated. Purification by flash column chromatography (using 100-200 mesh Silica gel, 4% MeOH/DCM as an eluent), followed by further purification by preparative HPLC gave a white solid (40 mg, 26%). ¹H NMR (300 MHz, DMSO-d₆): δ 9.55 (s, 1H), 8.18 (s, 1H), 7.41 (s, 1H), 7.33 (d, J=8.1 Hz, 1H), 7.23 (t, J=5.7 Hz, 1H), 7.13 (d, J=8.1 Hz, 1H), 6.56 (dd, J=1.8, 7.8 Hz, 1H), 4.33-4.27 (m, 1H), 3.42 (q, J=5.7 Hz, 2H), 3.36-3.32 (m, 2H), 3.22 (br s, 2H), 2.87 (d, J=10.2 Hz, 1H), 2.56-2.50 (m, 1H), 2.22 (t, J=6.0 Hz, 2H), 2.17 (s, 3H), 2.03-1.94 (m, 3H), 1.78-1.69 (m, 7H), 1.53-1.50 (m, 1H), 1.33-1.29 (m, 1H); LCMS (m/z): 507.5 [M+H]⁺.

### Example 9: 1-[3-[[5-cyclopropyl-2-[(2-methyl-3,4-dihydro-1H-isoquinolin-6-yl)amino]pyrimidin-4-yl]amino]propyl]pyrrolidin-2-one

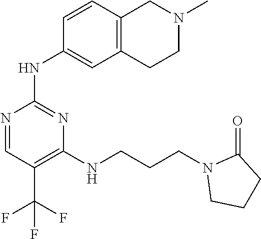 [SMILES: CN1CCc2cc(Nc3ncc(C(F)(F)F)c(NCCCN4CCCC4=O)n3)ccc2C1]

To a stirred solution of Intermediate 7 (160 mg, 0.54 mmol) in n-butanol (5 mL) was added Intermediate 4 (70.5 mg, 0.43 mmol) and followed by TFA (0.17 mL, 2.18 mmol) at room temperature. The reaction mixture was heated at 100° C. for 5 h and then evaporated under reduced pressure. The residue was suspended in water (20 mL), basified with aq. 1N NaOH solution and extracted with ethyl acetate (2×30 mL). The combined organic layers were washed with brine (30 mL), dried over anhydrous Na₂SO₄, filtered and the filtrate was concentrated. Purification by automated flash chromatography (gradient elution from 0-10% MeOH in DCM) gave a yellow solid (70 mg, 30%). ¹H NMR (400 MHz, CDCl₃): δ 7.67 (s, 1H), 7.46 (s, 1H), 7.30-7.27 (m, 1H), 6.92 (d, J=8.4 Hz, 2H), 6.38-6.44 (m, 1H), 3.53-3.48 (m, 4H), 3.43-3.39 (m, 4H), 2.90 (t, J=6.0 Hz, 2H), 2.67 (t, J=5.6 Hz, 2H), 2.45 (s, 3H), 2.44-2.42 (m, 2H), 2.10-2.03 (m, 2H), 1.84-1.78 (m, 2H), 1.51-1.48 (m, 1H), 0.94-0.89 (m, 2H), 0.51-0.47 (m, 2H); LCMS (m/z): 421.60 [M+H]⁺.

### Example 10: 1-(3-((5-Cyclopropyl-2-((2-methyl-1,2,3,4-tetrahydroisoquinolin-7-yl)amino)pyrimidin-4-yl)amino)propyl)pyrrolidin-2-one

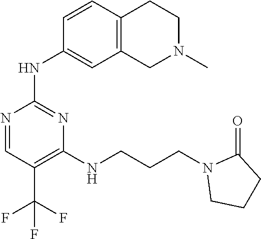 [SMILES: CN1CCc2ccc(Nc3ncc(C(F)(F)F)c(NCCCN4CCCC4=O)n3)cc2C1]

To a stirred solution of Intermediate 7 (160 mg, 0.54 mmol) in n-butanol (5 mL) was added Intermediate 5 (70.5 mg, 0.43 mmol) and followed by TFA (0.17 mL, 2.18 mmol) at room temperature. The reaction mixture was heated at 100° C. for 5 h and then evaporated under reduced pressure. The residue was suspended in water (20 mL), basified with aq. 1N NaOH solution and extracted with ethyl acetate (2×30 mL). The combined organic layer was washed with brine (30 mL), dried over anhydrous Na₂SO₄, filtered and the filtrate was concentrated. Purification by automated flash chromatography (gradient elution from 0-10% MeOH in DCM) gave a yellow solid (75 mg, 33%). ¹H NMR (400 MHz, CDCl₃): δ 7.67 (s, 1H), 7.38 (s, 1H), 7.30-7.28 (m, 1H), 7.01 (d, J=8.0 Hz, 1H), 6.85 (br s, 1H), 6.45-6.35 (m, 1H), 3.56 (s, 2H), 3.52-3.48 (m, 2H), 3.43-3.39 (m, 4H), 2.86 (t, J=5.6 Hz, 2H), 2.68 (t, J=5.6 Hz, 2H), 2.45-2.42 (m, 5H), 2.10-2.03 (m, 2H), 1.83-1.78 (m, 2H), 1.51-1.48 (m, 1H), 0.93-0.89 (m, 2H), 0.51-0.49 (m, 2H); LCMS (m/z): 421.60 [M+H]⁺.

### Example 11: 1-(3-((5-Cyclopropyl-2-((2-methyl-1,2,3,4-tetrahydroisoquinolin-6-yl)amino)pyrimidin-4-yl)amino)propyl)piperidin-2-one

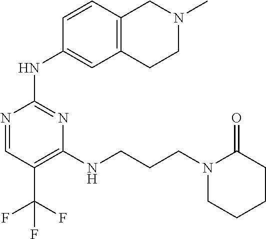 [SMILES: CN1CCc2cc(Nc3ncc(C(F)(F)F)c(NCCCN4CCCCC4=O)n3)ccc2C1]

To a solution of Intermediate 8 (170 mg, 0.55 mmol) in n-butanol (6 mL) was added trifluoroacetic acid (251 mg, 2.2 mmol) and Intermediate 4 (71.4 mg, 0.44 mmol) and the mixture was stirred at 100° C. for 3 h. The reaction mixture was evaporated, the residue was basified with 1N NaOH solution, extracted with ethyl acetate (2×20 mL). The combined organic layers were washed with brine, dried over anhydrous sodium sulfate, filtered and the filtrate was concentrated under reduced pressure. Trituration with diethyl ether gave an off-white solid (70 mg, 29%). ¹H NMR (400 MHz, CDCl₃): δ 7.67 (s, 1H), 7.46 (s, 1H), 7.29-7.25 (m, 1H), 6.92 (d, J=8.4 Hz, 1H), 6.72 (brs, 1H), 6.48 (brs, 1H), 3.52-3.47 (m, 6H), 3.29 (brs, 2H), 2.90 (t, J=6.0 Hz, 2H), 2.66 (t, J=6.0 Hz, 2H), 2.44-2.41 (m, 5H), 1.86-1.79 (m, 6H), 1.51-1.48 (m, 1H), 0.93-0.88 (m, 2H), 0.50-0.47 (m, 2H); LCMS (m/z): 435.5 (M+H)⁺

### Example 12: 1-(3-((5-Cyclopropyl-2-((2-methyl-1,2,3,4-tetrahydroisoquinolin-7-yl)amino)pyrimidin-4-yl)amino)propyl)piperidin-2-one

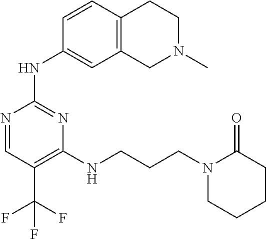 [SMILES: CN1CCc2ccc(Nc3ncc(C(F)(F)F)c(NCCCN4CCCCC4=O)n3)cc2C1]

Prepared analogously to Example 11 except using 2-methyl-1,2,3,4-tetrahydroisoquinolin-7-amine, Intermediate 5, (71.4 mg, 0.44 mmol). The product was isolated as a white solid (25 mg, 11%). ¹H NMR (300 MHz, DMSO-d₆): δ 8.71 (s, 1H), 7.59 (s, 1H), 7.53 (brs, 1H), 7.41 (d, J=8.4 Hz, 1H), 6.92 (d, J=8.4 Hz, 1H), 6.86-6.82 (m, 1H), 3.43-3.37 (m, 6H), 3.25-3.20 (m, 2H), 2.75-2.70 (m, 2H), 2.60-2.55 (m, 2H), 2.33 (s, 3H), 2.28-2.21 (m, 2H), 1.78-1.68 (m, 6H), 1.48-1.42 (m, 1H), 0.84-0.79 (m, 2H), 0.48-0.42 (m, 2H); LCMS (m/z): 435.52 (M+H)⁺

### Example 13: 1-(3-(5-Cyclopropyl-2-(3-((dimethyl amino) methyl) phenyl amino) pyrimidin-4-ylamino) propyl) piperidin-2-one

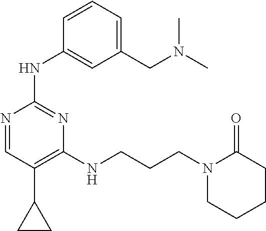 [SMILES: CN(C)Cc1cccc(Nc2ncc(C3CC3)c(NCCCN3CCCCC3=O)n2)c1]

To a stirred solution of Intermediate 8 (0.23 g, 0.74 mmol) in n-butanol (5 mL) was added 3-((dimethyl amino)methyl)aniline (0.11 g, 0.74 mmol) and TFA (0.31 g, 2.96 mmol) and the resulting solution was stirred at 100° C. for 5 h. The reaction mixture was concentrated, the residue was basified with saturated sodium bicarbonate solution (10 mL) and extracted with ethyl acetate (2×20 mL), and the combined organic layers were dried over anhydrous sodium sulfate, filtered and concentrated. Purification by preparative HPLC afforded a white solid (50 mg, 16%). ¹H NMR (300 MHz, DMSO-d₆): δ 8.82 (s, 1H), 7.80 (s, 1H), 7.60 (s, 1H), 7.56 (d, J=8.7 Hz, 1H), 7.13 (t, J=7.8 Hz, 1H), 6.86 (t, J=5.7 Hz, 1H), 6.76 (d, J=7.2 Hz, 1H), 3.46-3.35 (m, 5H), 3.24-3.22 (m, 2H), 2.22 (t, J=6.0 Hz, 2H), 2.14 (s, 6H), 1.81-1.70 (m, 6H), 1.48-1.43 (m, 1H), 0.85-0.79 (m, 2H), 0.49-0.44 (m, 2H); LCMS (m/z): 423.5 [M+H]⁺.

### Example 14: 1-(3-((5-Cyclopropyl-2-((4-(4-hydroxy-1-methylpiperidin-4-yl)phenyl)amino)pyrimidin-4-yl)amino)propyl)piperidin-2-one

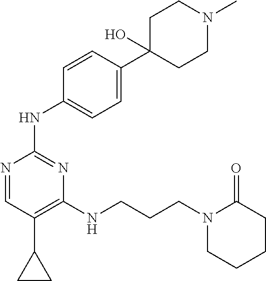 [SMILES: CN1CCC(O)(c2ccc(Nc3ncc(C4CC4)c(NCCCN4CCCCC4=O)n3)cc2)CC1]

A mixture of 4-(4-aminophenyl)-1-methylpiperidin-4-ol (Intermediate 9) (78.4 mg, 0.38 mmol), Intermediate 8 (150 mg, 0.48 mmol), sodium tert-butoxide (139 mg, 1.44 mmol), in 1,4-dioxane (10 mL) was degassed with argon for 5 minutes, prior to addition of Xantphos (22.2 mg, 0.038 mmol) and Pd₂(dba)₃ (26.4 mg, 0.028 mmol). The resulting reaction mixture was heated at 100° C. for 16 h. The reaction mixture was cooled to room temperature, filtered through Celite and washed with ethyl acetate. The filtrate was concentrated, and the residue was purified by flash column chromatography (gradient elution from 2-5% MeOH in DCM) gave an off-white solid (60 mg, 26%). ¹H NMR (400 MHz, DMSO-d₆): δ 8.77 (s, 1H), 7.67 (d, J=8.8 Hz, 2H), 7.59 (s, 1H), 7.29 (d, J=8.8 Hz, 2H), 6.83 (t, J=5.6 Hz, 1H), 4.55 (s, 1H), 3.39 (q, J=6.8 Hz, 4H), 3.23 (t, J=5.6 Hz, 2H), 2.50-2.49 (m, 2H), 2.32 (t, J=11.2 Hz, 2H), 2.23 (t, J=6.4 Hz, 2H), 2.18 (s, 3H), 1.92-1.86 (m, 2H), 1.79-1.69 (m, 6H), 1.55 (d, J=12.4 Hz, 2H), 1.46-1.44 (m, 1H), 0.84-0.79 (m, 2H), 0.47-0.43 (m, 2H); LCMS (m/z): 479.52 (M+H)⁺

### Example 15: 5-Cyclopropyl-N2-(2-methyl-1,2,3,4-tetrahydroisoquinolin-6-yl)-N4-(3-(pyrrolidin-1-yl)propyl)pyrimidine-2,4-diamine

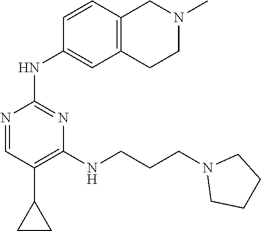 [SMILES: CN1CCc2cc(Nc3ncc(C4CC4)c(NCCCN4CCCC4)n3)ccc2C1]

### Step 1: Synthesis of 2-chloro-5-cyclopropyl-N-(3-(pyrrolidin-1-yl)propyl)pyrimidin-4-amine

To a solution of 2,4-dichloro-5-cyclopropylpyrimidine (500 mg, 2.64 mmol) in IPA (10 mL) was added DIPEA (1.38 mL, 7.92 mmol) and 3-(pyrrolidin-1-yl)propan-1-amine (508 mg, 3.96 mmol) at 0° C. and the mixture was then heated to 50° C. for 16 h. The reaction mixture was evaporated; the residue was taken in ethyl acetate (30 mL) and washed with water (30 mL) and the organic layer was dried over anhydrous sodium sulfate, filtered and the filtrate was concentrated. The crude was purified by flash column chromatography (silica gel, 100-200 mesh, eluted with 50% ethyl acetate/pet ether) to afford a white solid (400 mg, 53.8%).

### Step 2: Synthesis of 5-cyclopropyl-N2-(2-methyl-1,2,3,4-tetrahydroisoquinolin-6-yl)-N4-(3-(pyro-lidin-1-yl)propyl)pyrimidine-2,4-diamine

To a solution of 2-chloro-5-cyclopropyl-N-(3-(pyrrolidin-1-yl)propyl)pyrimidin-4-amine (200 mg, 0.71 mmol) in tert-butanol (6 mL) was added TFA (324 mg, 2.84 mmol) and Intermediate 4 (91 mg, 0.56 mmol) at room temperature. The reaction mixture was evaporated, the residue was basified with 1N NaOH solution, extracted with ethyl acetate (2×20 mL), the combined organic layers were washed with brine, dried over anhydrous sodium sulfate, filtered and concentrated under reduced pressure. The crude solid was purified by triturating with diethyl ether to afford an off-white solid (70 mg, 27.6%). ¹H NMR (400 MHz, DMSO-d₆): δ 8.68 (s, 1H), 7.61 (s, 1H), 7.57 (s, 1H), 7.41 (dd, J1=1.6, J2=8.0 Hz, 1H), 6.88 (t, J=5.2 Hz, 1H), 6.84 (d, J=8.0 Hz, 1H), 3.48 (q, J=6.8 Hz, 2H), 3.37 (s, 2H), 2.75 (t, J=6.0 Hz, 2H), 2.54 (t, J=6.0 Hz, 2H), 2.43 (brs, 6H), 2.30 (s, 3H), 1.80-1.76 (m, 2H), 1.66 (brs, 4H), 1.43-1.40 (m, 1H), 0.81-0.76 (m, 2H), 0.46-0.42 (m, 2H); LCMS (m/z): 407.5 (M+H)⁺

### Example 16: 1-(3-((5-Cyclopropyl-2-((3-((1-methylpiperidin-4-yl)oxy)phenyl)amino)pyrimidin-4-yl)amino)propyl)piperidin-2-one

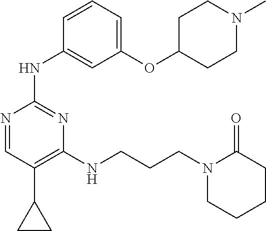 [SMILES: CN1CCC(Oc2cccc(Nc3ncc(C4CC4)c(NCCCN4CCCCC4=O)n3)c2)CC1]

To a stirred solution of Intermediate 8 (0.17 g, 0.55 mmol) in n-butanol (5 mL) was added 3-((1-methylpiperidin-4-yl)oxy)aniline (0.1 g, 0.55 mmol) and TFA (0.23 g, 2.2 mmol) at room temperature and the resulting solution was stirred at 100° C. for 5 h. The reaction mixture was cooled to room temperature and concentrated; the residue was basified with saturated sodium bicarbonate solution (10 mL) and extracted with ethyl acetate (2×20 mL). The combined organic layers were dried over anhydrous Na₂SO₄, filtered and the filtrate was concentrated. The crude was purified by preparative HPLC to afford a white solid (90 mg, 37%). ¹H NMR (300 MHz, CDCl₃): δ 7.69 (s, 1H), 7.32 (d, J=1.8 Hz, 1H), 7.15-7.11 (m, 2H), 6.80 (br s, 1H), 6.56-6.44 (m, 2H), 4.40-4.34 (m, 1H), 3.53-3.49 (m, 4H), 3.29 (br s, 2H), 2.96 (d, J=8.4 Hz, 1H), 2.63-2.59 (m, 1H), 2.43 (br s, 2H), 2.29 (s, 3H), 2.18-1.99 (m, 3H), 1.84-1.79 (m, 7H), 1.60-1.44 (m, 3H), 0.95-0.89 (m, 2H), 0.52-0.47 (m, 2H); LCMS (m/z): 479.58 [M+H]⁺

### Example 17: N4,5-dicyclopropyl-N2-(2-methyl-1,2,3,4-tetrahydroisoquinolin-6-yl)pyrimidine-2,4-diamine

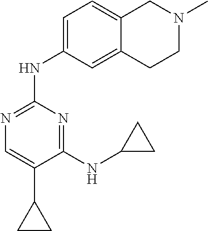 [SMILES: CN1CCc2cc(Nc3ncc(C4CC4)c(NC4CC4)n3)ccc2C1]

To a solution of Intermediate 10 (0.2 g, 0.95 mmol) in t-butanol (4 mL) was added TFA (0.43 g, 3.82 mmol) and Intermediate 4 (0.12 g, 0.76 mmol) at room temperature and stirred at 100° C. for 3 h. The reaction mixture was evaporated, the residue was basified with 1N NaOH solution, extracted with ethyl acetate (2×20 mL). The combined organic layers were washed with brine, dried over anhydrous sodium sulfate, filtered and concentrated under reduced pressure. The crude solid was purified by triturating with diethyl ether to afford an off-white solid (0.05 g, 16%). ¹H NMR (300 MHz, DMSO-d₆): δ 8.75 (s, 1H), 7.79 (s, 1H), 7.57 (s, 1H), 7.49 (dd, J₁=1.8 Hz, J₂=8.4 Hz, 1H), 6.84 (d, J=8.4 Hz, 1H), 6.74 (d, J=2.7 Hz, 1H), 3.37 (s, 2H), 2.85-2.82 (m, 1H), 2.76-2.72 (m, 2H), 2.56-2.49 (m, 2H), 2.30 (s, 3H), 1.49-1.44 (m, 1H), 0.80-0.76 (m, 4H), 0.64-0.59 (m, 2H), 0.46-0.41 (m, 2H); LCMS (m/z): 336.19 [M+H]⁺

### Example 18: N4,5-dicyclopropyl-N2-(2-methyl-1,2,3,4-tetrahydroisoquinolin-7-yl)pyrimidine-2,4-diamine

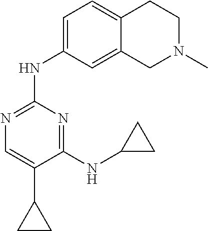 [SMILES: CN1CCc2ccc(Nc3ncc(C4CC4)c(NC4CC4)n3)cc2C1]

To a solution of Intermediate 10 (0.2 g, 0.95 mmol) in t-butanol (4 mL) was added TFA (0.43 g, 3.82 mmol) and 2-methyl-1,2,3,4-tetrahydroisoquinolin-7-amine (0.12 g, 0.76 mmol) at room temperature and the mixture was stirred at 100° C. for 3 h. The reaction mixture was evaporated, the residue was basified with 1N NaOH solution, extracted with ethyl acetate (2×20 mL). The combined organic layers were washed with brine, dried over anhydrous sodium sulfate, filtered concentrated under reduced pressure. The crude solid was purified by triturating with diethyl ether to afford the product as an off-white solid (0.06 g, 18.7%). ¹H NMR (300 MHz, DMSO-d₆): δ ppm 8.79 (s, 1H), 7.72 (s, 1H), 7.57-7.51 (m, 2H), 6.93 (d, J=8.4 Hz, 1H), 6.76 (d, J=2.4 Hz, 1H), 3.52 (s, 2H), 2.86-2.82 (m, 1H), 2.81-2.68 (m, 4H), 2.40 (s, 3H), 1.49-1.42 (m, 1H), 0.80-0.74 (m, 4H), 0.69-0.59 (m, 2H), 0.46-0.41 (m, 2H); LCMS (m/z): 336.19 [M+H]⁺.

### Example 19: 1-[3-[[2-(1,2,3,4-Tetrahydroisoquinolin-6-ylamino)-5-(trifluoromethyl)pyrimidin-4-yl]amino]propyl]piperidin-2-one

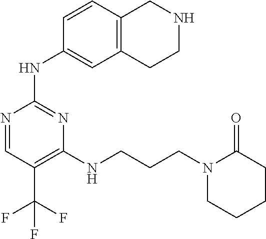 [SMILES: O=C1CCCCN1CCCNc1nc(Nc2ccc3c(c2)CCNC3)ncc1C(F)(F)F]

Intermediate 1 (0.050 g, 0.149 mmol) and tert-butyl 6-amino-3,4-dihydro-1H-isoquinoline-2-carboxylate (0.039 g, 0.156 mmol) were combined in IPA (2 mL) and heated at 80° C. for 3 hours. The reaction mixture was cooled, concentrated in vacuo onto silica and purified via column chromatography (10-100% EtOAc in PE). The fractions containing product were combined and concentrated in vacuo. The product was dissolved in DCM and TFA added, and the resulting solution stirred for 15 minutes. The reaction mixture was passed through an SCX cartridge and the product eluted with 2M NH₃ in MeOH. The eluent was concentrated to give the product, 1-[3-[[2-(1,2,3,4-tetrahydroisoquinolin-6-ylamino)-5-(trifluoromethyl)pyrimidin-4-yl]amino]propyl]piperidin-2-one (0.044 g, 0.098 mmol, 66% Yield), as an off-white solid. ¹H NMR (400 MHz, DMSO-d6) δ ppm 9.50 (s, 1H), 8.17 (s, 1H), 7.51-7.58 (m, 1H), 7.38-7.44 (m, J=1.00, 1.00 Hz, 1H), 7.21-7.23 (m, 1H), 7.19-7.26 (m, 1H), 6.95 (d, J=8.70 Hz, 1H), 3.85 (s, 2H), 3.40-3.44 (m, 2H), 3.23 (s, 3H), 2.96-3.03 (m, 2H), 2.66-2.73 (m, 2H), 2.19-2.25 (m, 2H), 1.67-1.77 (m, 6H); LCMS (m/z): 449.1 [M+H]⁺.

### Examples 20-23

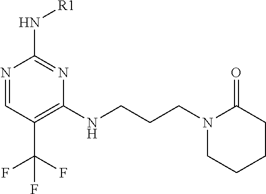

Examples 20-23 were prepared analogously to Example 19 from Intermediate 1 and the appropriate amine:

| Example # | R1 | IUPAC name | 1H NMR |
|---|---|---|---|
| 20 | 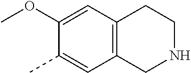 | 1-[3-[[2-[(6-Methoxy-1,2,3,4- tetrahydroisoquinolin-7- yl)amino]-5- (trifluoromethyl)pyrimidin-4- yl]amino]propyl]piperidin-2-one | ¹H NMR (400 MHz, CHLOROFORM-d) d 8.11-8.17 (m, 2H), 7.57-7.65 (m, 1H), 6.73-6.81 (m, 1H), 6.58 (s, 1H), 4.00 (s, 2H), 3.82-3.87 (m, 3H), 3.49-3.54 (m, 2H), 3.43-3.48 (m, 2H), 3.29 (br. s., 2H), |
| | | | 3.12-3.20 (m, 2H), 2.74-2.81 (m, 2H), |
| | | | 2.42-2.47 (m, 2H), 1.78-1.87 (m, 6H); |
| | | | LCMS (m/z): 479.1 [M + H]⁺. |
| 21 | 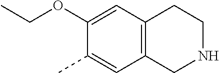 | 1-[3-[[2-[(6-Ethoxy-1,2,3,4- tetrahydroisoquinolin-7- yl)amino]-5- (trifluoromethyl)pyrimidin-4- yl]amino]propyl]piperidin-2-one | ¹H NMR (400 MHz, CHLOROFORM-d) d 8.19 (s, 1H), 8.14 (s, 1H), 7.64 (s, 1H), 6.69-6.76 (m, 1H), 6.58 (s, 1H), 4.08 (q, J = 7.17 Hz, 2H), 4.03 (s, 2H), 3.50-3.56 (m, 2H), 3.44-3.49 (m, 2H), 3.29 (t, J = |
| | | | 5.27 Hz, 2H), 3.20 (t, J = 5.95 Hz, 2H), |
| | | | 2.77-2.83 (m, 2H), 2.40-2.47 (m, 2H), |
| | | | 1.77-1.86 (m, 6H), 1.46 (t, J = 6.87 Hz, |
| | | | 3H); LCMS (m/z): 493.1 [M + H]⁺. |
| 22 | 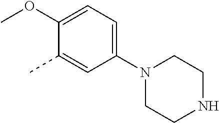 | 4-[4-methoxy-3-[[4-[3-(2-oxo-1- piperidyl)propylamino]-5- (trifluoromethyl)pyrimidin-2- yl]amino]phenyl]piperazine | ¹H NMR (400 MHz, DMSO-d6) d 8.16 (s, 1H), 7.93 (s, 1H), 7.82 (d, J = 2.75 Hz, 1H), 7.25-7.32 (m, 1H), 6.90 (d, J = 8.70 Hz, 1H), 6.55-6.60 (m, 1H), 3.78 (s, 3H), 3.39-3.45 (m, 2H), 3.27-3.31 (m, 2H), 3.17-3.22 (m, 2H), 2.90-2.96 (m, 4H), 2.80-2.86 (m, 4H), 2.18-2.25 (m, 2H), 1.65-1.73 (m, 6H); LCMS (m/z): |
| | | | 508.1 [M + H]⁺. |
| 23 | 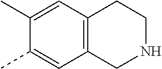 | 6-methyl-7-[[4-[3-(2-oxo-1- piperidyl)propylamino]-5- (trifluoromethyl)pyrimidin-2- yl]amino]-3,4-dihydro-1H- isoquinoline | ¹H NMR (400 MHz, CHLOROFORM-d) d 8.12 (s, 1H), 7.64-7.72 (m, 1H), 6.93 (s, 1H), 6.68-6.78 (m, 2H), 4.00 (s, 2H), 3.42-3.48 (m, 4H), 3.24-3.31 (m, 2H), 3.14 (t, J = 5.95 Hz, 2H), 2.72-2.78 (m, |
| | | | 2H), 2.41-2.49 (m, 2H), 2.26 (s, 3H), |
| | | | 1.74-1.85 (m, 6H); LCMS (m/z): 463.1 |
| | | | [M + H]⁺. |

### Example 24: 1-[3-[[2-[(6-Methoxy-2-methyl-3,4-dihydro-1H-isoquinolin-7-yl)amino]-5-(trifluoromethyl)pyrimidin-4-yl]amino]propyl]piperidin-2-one

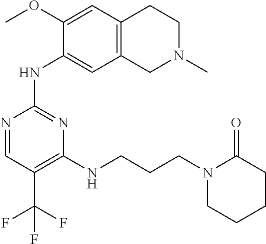 [SMILES: COc1cc2c(cc1Nc1ncc(C(F)(F)F)c(NCCCN3CCCCC3=O)n1)CN(C)CC2]

To a solution of 1-[3-[[2-[(6-methoxy-1,2,3,4-tetrahydroisoquinolin-7-yl)amino]-5-(trifluoromethyl)-pyrimidin-4-yl]amino]propyl]piperidin-2-one (0.110 g, 0.230 mmol) in DCM was added formaldehyde (22 mL, 0.276 mmol) followed by sodium triacetoxyborohydride (0.098 g, 0.460 mmol), and the reaction mixture left to stir for 1 hour. The reaction mixture was diluted with DCM and saturated NaHCO₃ (aq.), the organic layer separated, dried and concentrated in vacuo to give a brown gum. The gum was taken up in MeOH and submitted for preparative HPLC, to give the product (0.033 g, 29%). ¹H NMR (400 MHz, DMSO-d6) d 8.14 (s, 1H), 7.97 (s, 1H), 7.76 (s, 1H), 7.20-7.31 (m, 1H), 6.76 (s, 1H), 3.80 (s, 3H), 3.42 (s, 2H), 3.35-3.39 (m, 2H), 3.30 (t, J=6.64 Hz, 2H), 3.21 (t, J=5.50 Hz, 2H), 2.78 (s, 2H), 2.57 (t, J=5.95 Hz, 2H), 2.33 (s, 3H), 2.22 (t, J=6.18 Hz, 2H), 1.65-1.76 (m, 6H); LCMS (m/z): 493.1 [M+H]⁺.

### Example 25: 1-[3-[[2-(Isochroman-7-ylamino)-5-(trifluoromethyl)pyrimidin-4-yl]amino]propyl]piperidin-2-one

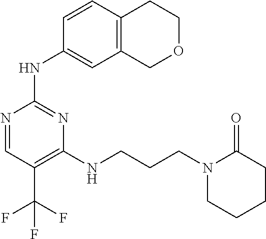 [SMILES: O=C1CCCCN1CCCNc1nc(Nc2ccc3c(c2)COCC3)ncc1C(F)(F)F]

Intermediate 1 (0.050 g, 0.149 mmol) and isochroman-7-amine (0.022 g, 0.149 mmol) were combined in IPA, sealed in a microwave vial and heated at 80° C. for 2 hours. The reaction mixture was cooled and concentrated in vacuo onto silica and purified via column chromatography (10-100% EtOAc in PE). The fractions containing product were combined and concentrated to give the product, 1-[3-[[2-(isochroman-7-ylamino)-5-(trifluoromethyl)pyrimidin-4-yl]amino]propyl]piperidin-2-one (0.011 g, 0.024 mmol, 16% Yield), as an off-white solid. ¹H NMR (400 MHz, DMSO-d6) d 9.57 (br. s, 1H), 8.16 (s, 1H), 7.48-7.53 (m, 1H), 7.39-7.45 (m, 1H), 7.17-7.25 (m, 1H), 7.01-7.07 (m, 1H), 4.65 (s, 2H), 3.82-3.89 (m, 2H), 3.40 (q, J=6.11 Hz, 2H), 3.29-3.33 (m, 2H), 3.22 (s, 2H), 2.68-2.74 (m, 2H), 2.18-2.27 (m, 2H), 1.63-1.78 (m, 6H). LCMS (m/z): 450.0 [M+H]⁺.

### Examples 26-37

Examples 26-37 were prepared analogously to Example 25 from Intermediate 1 and the appropriate amine

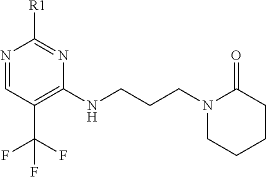

| Example | | | |
|---|---|---|---|
| # | R1 | Name | Analytical Data |
| 26 | 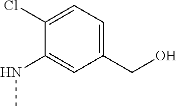 | 1-[3-[[2-[2-Chloro-5- (hydroxymethyl)anilino]-5- (trifluoromethyl)pyrimidin-4- yl]amino]propyl]piperidin-2-one | ¹H NMR (400 MHz, DMSO-d6) d 8.91-8.97 (m, 1H), 8.16 (s, 1H), 7.74- 7.77 (m, 1H), 7.35-7.46 (m, 2H), 7.10-7.14 (m, 1H), 4.48 (s, 2H), 3.26-3.32 (m, 2H), 3.20-3.25 (m, 2H), 3.14-3.19 (m, 2H), 2.16-2.24 (m, 2H), 1.57-1.73 (m, 6H); LCMS |
| | | | (m/z): 458 [M + H]⁺. |
| 27 | 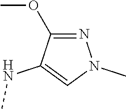 | 1-[3-[[2-[(3-Methoxy-1-methyl- pyrazol-4-yl)amino]-5- (trifluoromethyl)pyrimidin-4- yl]amino]propyl]piperidin-2-one | ¹H NMR (400 MHz, CHLOROFORM-d) d 9.87 (s, 1H), 8.54-8.65 (m, 1H), 7.90 (s, 1H), 7.37 (s, 1H), 3.92 (s, 3H), 3.75 (s, 3H), 3.41-3.51 (m, 4H), 3.28-3.34 (m, 2H), 2.43-2.50 (m, 2H), 1.81-1.89 (m, 4H), 1.73-1.80 |
| | | | (m, 2H); LCMS (m/z): 428 [M + H]⁺. |
| 28 | 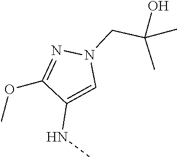 | 1-[3-[[2-[1-(2-Hydroxy-2- methyl-propyl)-3-methoxy- pyrazol-4-yl]amino]-5- (trifluoromethyl)pyrimidin-4- yl]amino]propyl]piperidin-2-one | ¹H NMR (400 MHz, DMSO-d6) d 8.55-8.64 (m, 1H), 8.07 (s, 1H), 7.60- 7.73 (m, 1H), 7.06-7.16 (m, 1H), 4.56-4.70 (m, 1H), 3.82 (br. s., 2H), 3.78 (s, 3H), 3.16-3.32 (m, 6H), 2.17- 2.26 (m, 2H), 1.60-1.76 (m, 6H), 1.06 (s, 6H); LCMS (m/z): 486 [M + H]⁺. |
| 29 | 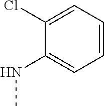 | 1-[3-[[2-[(6-Chlorocyclohexa- 1,5-dien-1-yl)amino]-5- (trifluoromethyl)pyrimidin-4- yl]amino]propyl]piperidin-2-one | ¹H NMR (400 MHz, CHLOROFORM-d) d 8.45-8.51 (m, 1H), 8.16-8.21 (m, 1H), 7.47-7.60 (m, 1H), 7.36-7.42 (m, 1H), 7.23- 7.30 (m, 1H), 6.94-7.01 (m, 1H), 6.81-6.89 (m, 1H), 3.43-3.54 (m, 4H), 3.25-3.32 |
| | | | (m, 2H), 2.40-2.49 (m, 2H), 1.77- |
| | | | 1.87 (m, 6H); LCMS (m/z): 428 |
| | | | [M + H]⁺. |
| 30 | 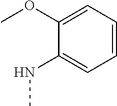 | 1-[3-[[2-(2-Methoxyanilino)-5- (trifluoromethyl)pyrimidin-4- yl]amino]propyl]piperidin-2-one | ¹H NMR (400 MHz, CHLOROFORM-d) d 8.42-8.48 (m, 1H), 8.13-8.18 (m, 1H), 7.65-7.75 (m, 1H), 6.94-7.02 (m, 2H), 6.87- 6.92 (m, 1H), 6.66-6.76 (m, 1H), 3.90 (s, 3H), 3.52-3.59 (m, 2H), 3.45- 3.49 (m, 2H), 3.25-3.32 (m, 2H), |
| | | | 2.41-2.48 (m, 2H), 1.78-1.87 (m, |
| | | | 6H); LCMS (m/z): 424 [M + H]⁺. |
| 31 | 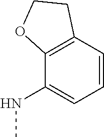 | 1-[3-[[2-(2,3- Dihydrobenzofuran-7-ylamino)- 5-(trifluoromethyl)pyrimidin-4- yl]amino]propyl]piperidin-2-one | ¹H NMR (400 MHz, CHLOROFORM-d) d 8.14-8.17 (m, 1H), 8.09-8.13 (m, 1H), 7.21-7.27 (m, 1H), 6.87-6.91 (m, 1H), 6.82- 6.87 (m, 1H), 6.67-6.74 (m, 1H), 4.63 (t, J = 8.70 Hz, 2H), 3.44-3.55 (m, 4H), 3.22- 3.34 (m, 4H), 2.40-2.49 (m, 2H), 1.74-1.88 (m, 6H); LCMS (m/z): 436 |
| | | | [M + H]⁺. |
| 32 | 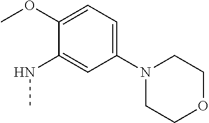 | 1-[3-[[2-(2-Methoxy-5- morpholino-anilino)-5- (trifluoromethyl)pyrimidin-4- yl]amino]propyl]piperidin-2-one | ¹H NMR (400 MHz, DMSO-d6) d 8.17 (s, 1H), 7.93 (s, 1H), 7.82 (d, J = 2.75 Hz, 1H), 7.25-7.32 (m, 1H), 6.90 (d, J = 8.70 Hz, 1H), 6.54-6.61 (m, 1H), 3.78 (s, 3H), 3.39-3.45 (m, 2H), 3.27- 3.32 (m, 2H), 3.16-3.23 (m, 2H), 2.90-2.96 (m, 4H), 2.80-2.87 (m, |
| | | | 4H), 2.17-2.26 (m, 2H), 1.63-1.75 |
| | | | (m, 6H); LCMS (m/z): 509 [M + H]⁺. |
| 33 | 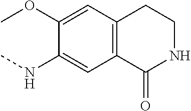 | 6-Methoxy-7-[[4-[3-(2-oxo-1- piperidyl)propylamino]-5- (trifluoromethyl)pyrimidin-2- yl]amino]-3,4-dihydro-2H- isoquinolin-1-one | ¹H NMR (400 MHz, CHLOROFORM-d) d 9.25-9.30 (m, 1H), 8.13 (s, 1H), 7.65-7.70 (m, 1H), 6.66-6.70 (m, 1H), 3.95 (s, 3H), 3.66- 3.71 (m, 2H), 3.54-3.60 (m, 2H), 3.45-3.51 (m, 2H), 3.27-3.35 (m, 2H), 2.93-3.00 (m, 2H), 2.41-2.48 |
| | | | (m, 2H), 1.91-1.98 (m, 2H), 1.78- |
| | | | 1.85 (m, 4H); LCMS (m/z): 493 |
| | | | [M + H]⁺. |
| 34 | 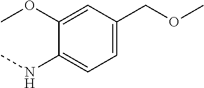 | 1-[3-[[2-[2-Methoxy-4- (methoxymethyl)anilino]-5- (trifluoromethyl)pyrimidin-4- yl]amino]propyl]piperidin-2-one | ¹H NMR (400 MHz, CHLOROFORM-d) d 8.38-8.46 (m, 1H), 8.16 (d, J = 0.92 Hz, 1H), 7.68- 7.78 (m, 1H), 6.88-6.94 (m, 2H), 6.69-6.77 (m, 1H), 4.43 (s, 2H), 3.92 (s, 3H), 3.51-3.58 (m, 2H), 3.45-3.50 |
| | | | (m, 2H), 3.40 (s, 3H), 3.25-3.33 (m, |
| | | | 2H), 2.41-2.49 (m, 2H), 1.80-1.89 |
| | | | (m, 6H); LCMS (m/z): 468 [M + H]⁺. |
| 35 | 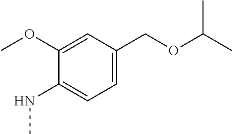 | 1-[3-[[2-[4-(Isopropoxymethyl)- 2-methoxy-anilino]-5- (trifluoromethyl)pyrimidin-4- yl]amino]propyl]piperidin-2-one | ¹H NMR (400 MHz, CHLOROFORM-d) d 8.37-8.41 (m, 1H), 8.12-8.17 (m, 1H), 7.68-7.75 (m, 1H), 6.89-6.95 (m, 2H), 6.66- 6.75 (m, 1H), 4.48 (s, 2H), 3.91 (s, 3H), 3.70 (spt, J = 6.10 Hz, 1H), 3.51- 3.58 (m, 2H), 3.44-3.49 (m, 2H), 3.27-3.32 (m, 2H), 2.41-2.49 (m, 2H), 1.80-1.88 (m, 6H), 1.23 (d, |
| | | | J = 6.10 Hz, 6H); LCMS (m/z): 496 |
| | | | [M + H]⁺. |
| 36 | 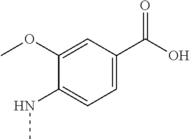 | 3-Methoxy-4-[[4-[3-(2-oxo-1- piperidyl)propylamino]-5- (trifluoromethyl)pyrimidin-2- yl]amino]benzoic acid | ¹H NMR (400 MHz, DMSO-d6) d 8.96-9.19 (m, 1H), 8.36-8.45 (m, 1H), 8.19-8.26 (m, 1H), 8.06-8.18 (m, 1H), 7.59-7.63 (m, 1H), 7.50-7.54 (m, 1H), 3.92 (s, 3H), 3.40 (q, J = 6.41 Hz, 2H), 3.23-3.31 (m, 2H), 3.13-3.20 (m, 2H), 2.13-2.20 (m, 2H), 1.69-1.77 (m, 2H), 1.58- 1.68 (m, 4H); LCMS (m/z): 468 |
| | | | [M + H]⁺. |
| 37 | 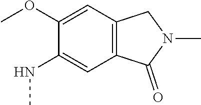 | 5-Methoxy-2-methyl-6-[[4-[3- (2-oxo-1- piperidyl)propylamino]-5- (trifluoromethyl)pyrimidin-2- yl]amino]isoindolin-1-one | ¹H NMR (400 MHz, METHANOL- D3) d 8.23-8.36 (m, 2H), 7.31-7.36 (m, 1H), 4.48 (s, 2H), 3.98-4.02 (m, 3H), 3.55-3.60 (m, 2H), 3.38-3.44 (m, 2H), 3.26-3.28 (m, 2H), 3.17 (s, 3H), 2.25-2.36 (m, 2H), 1.84-1.93 (m, 2H), 1.69-1.81 (m, |
| | | | 4H); LCMS (m/z): 493 [M + H]⁺. |

### Example 38: 1-[3-[[2-[2-Methoxy-4-(morpholine-4-carbonyl)anilino]-5-(trifluoromethyl)pyrimidin-4-yl]amino]propyl]piperidin-2-one

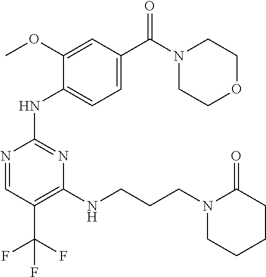 [SMILES: COc1cc(C(=O)N2CCOCC2)ccc1Nc1ncc(C(F)(F)F)c(NCCCN2CCCCC2=O)n1]

To 3-methoxy-4-[[4-[3-(2-oxo-1-piperidyl)propylamino]-5-(trifluoromethyl)pyrimidin-2-yl]amino]ben-zoic acid (0.050 g, 0.107 mmol) in DMF (1 mL) was added HATU (0.049 g, 0.128 mmol), DIPEA (0.039 mL, 0.214 mmol) and morpholine (0.010 g, 0.118 mmol), and the reaction mixture stirred overnight at room temperature. The reaction mixture was diluted with EtOAc and water, the organic layer separated, washed with water, and concentrated in vacuo. The residue was taken up in MeOH and submitted for preparative HPLC to give the product (0.022 g, 38%). ¹H NMR (400 MHz, DMSO-d6) d 8.23 (d, J=8.24 Hz, 1H), 8.19 (s, 1H), 8.12 (s, 1H), 7.30-7.36 (m, 1H), 7.07-7.09 (m, 1H), 7.02-7.06 (m, 1H), 3.89 (s, 3H), 3.44-3.67 (m, 8H), 3.35-3.41 (m, 2H), 3.28-3.32 (m, 2H), 3.19-3.24 (m, 2H), 2.19-2.25 (m, 2H), 1.65-1.77 (m, 6H); LCMS (m/z): 537 [M+H]⁺.

### Examples 39-41

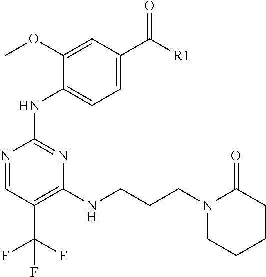

Examples 39-41 were prepared analogously to Example 38 from 3-methoxy-4-[[4-[3-(2-oxo-1-piperidyl)propylamino]-5-(trifluoromethyl)pyrimidin-2-yl]amino]benzoic acid and the appropriate amine.

| Example | | | |
|---|---|---|---|
| # | R1 | IUPAC name | Analytical data |
| 39 | —NHMe | 3-Methoxy-N-methyl-4-[[4-[3-(2- | ¹H NMR (400 MHz, DMSO-d6) d |
| | | oxo-1-piperidyl)propylamino]-5- | 8.29-8.36 (m, 1H), 8.20-8.25 (m, 1H), |
| | | (trifluoromethyl)pyrimidin-2- | 8.16-8.19 (m, 1H), 8.06-8.10 (m, 1H), |
| | | yl]amino]benzamide | 7.42-7.48 (m, 2H), 7.29-7.34 (m, 1H), |
| | | | 3.88 (s, 3H), 3.34-3.38 (m, 2H), 3.25- |
| | | | 3.28 (m, 2H), 3.15-3.21 (m, 2H), 2.72- |
| | | | 2.77 (m, 3H), 2.16-2.21 (m, 2H), 1.62- |
| | | | 1.74 (m, 6H); LCMS (m/z): 481 |
| | | | [M + H]⁺. |
| 40 | —NMe₂ | 3-Methoxy-N,N-dimethyl-4-[[4-[3- | ¹H NMR (400 MHz, DMSO-d6) d |
| | | (2-oxo-1-piperidyl)propylamino]-5- | 8.14-8.20 (m, 2H), 8.08 (s, 1H), 7.24- |
| | | (trifluoromethyl)pyrimidin-2- | 7.31 (m, 1H), 7.02-7.05 (m, 1H), 6.96- |
| | | yl]amino]benzamide | 7.01 (m, 1H), 3.84 (s, 3H), 3.31-3.37 |
| | | | (m, 2H), 3.24-3.29 (m, 2H), 3.15-3.20 |
| | | | (m, 2H), 2.93 (s, 6H), 2.15-2.22 (m, |
| | | | 2H), |
| | | | 1.61-1.73 (m, 6H); LCMS (m/z): 495 |
| | | | [M + H]⁺. |
| 41 | 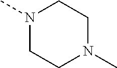 | 1-[3-[[2-[2-Methoxy-4-(4- methylpiperazine-1- carbonyl)anilino]-5- (trifluoromethyl)pyrimidin-4- yl]amino]propyl]piperidin-2-one | ¹H NMR (400 MHz, DMSO-d6) d 8.17-8.21 (m, 1H), 8.16 (d, J = 0.92 Hz, 1H), 8.08 (s, 1H), 7.24-7.32 (m, 1H), 7.01 (d, J = 1.83 Hz, 1H), 6.98 (dd, J = 1.83, 8.24 Hz, 1H), 3.85 (s, 3H), 3.32-3.57 (m, 6H), 3.24-3.28 (m, 2H), 3.18 (t, J = 5.50 Hz, 2H), 2.12-2.42 (m, 9H), 1.60-1.74 (m, 6H); LCMS (m/z): 550 [M + H]⁺. |

### Example 42: 3-Methoxy-4-[[4-[3-(2-oxo-1-piperidyl)propylamino]-5-(trifluoromethyl)pyrimidin-2-yl]amino]-N-(4-piperidyl)benzamide

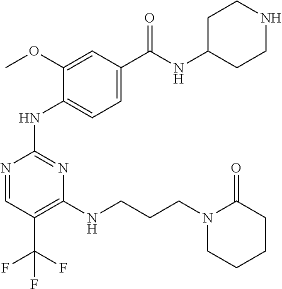 [SMILES: COc1cc(C(=O)NC2CCNCC2)ccc1Nc1ncc(C(F)(F)F)c(NCCCN2CCCCC2=O)n1]

Prepared in a similar manner to Example 38 from 3-methoxy-4-[[4-[3-(2-oxo-1-piperidyl)-propylamino]-5-(trifluoromethyl)pyrimidin-2-yl]amino]benzoic acid and tert-butyl 4-aminopiperidine-1-carboxylate except the removal of the BOC-group was carried out by stirring the crude product in 1:1 DCM-TFA (2 mL) for 30 minutes. The reaction mixture was concentrated in vacuo and purified by preparative HPLC. ¹H NMR (400 MHz, DMSO-d6) d 8.23 (d, J=8.24 Hz, 1H), 8.18 (d, J=0.92 Hz, 1H), 8.13-8.17 (m, 1H), 8.09 (s, 1H), 7.46-7.50 (m, 1H), 7.45 (d, J=1.83 Hz, 1H), 7.30-7.35 (m, 1H), 3.89 (s, 3H), 3.80-3.86 (m, 1H), 3.33-3.40 (m, 2H), 3.25-3.29 (m, 2H), 3.15-3.21 (m, 2H), 2.96-3.04 (m, 2H), 2.54-2.62 (m, 2H), 2.19 (s, 2H), 1.61-1.78 (m, 8H), 1.38-1.51 (m, 2H); LCMS (m/z): 550 [M+H]⁺

### Example 43: 1-[3-[[2-[(6-Chloro-1,2,3,4-tetrahydroisoquinolin-7-yl)amino]-5-(trifluoromethyl)pyrimidin-4-yl]amino]propyl]piperidin-2-one

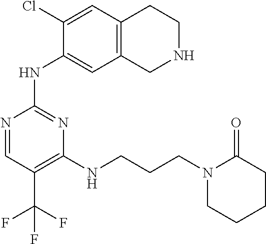 [SMILES: O=C1CCCCN1CCCNc1nc(Nc2cc3c(cc2Cl)CCNC3)ncc1C(F)(F)F]

Prepared analogously to Example 19 from Intermediate 1 and Intermediate 17. ¹H NMR (400 MHz, DMSO-d6) d ppm 8.74 (s, 1H), 8.10 (s, 1H), 7.40 (s, 1H), 7.15-7.23 (m, 2H), 3.83 (s, 2H), 3.21-3.29 (m, 4H), 3.15-3.20 (m, 2H), 2.91-2.98 (m, 2H), 2.64-2.71 (m, 2H), 2.21 (s, 2H), 1.59-1.74 (m, 6H); LCMS (m/z): 483/485 [M+H⁺].

### Example 44: 1-[3-[[2-[2-Chloro-5-(4-piperidyloxy)anilino]-5-(trifluoromethyl)pyrimidin-4-yl]amino]propyl]piperidin-2-one

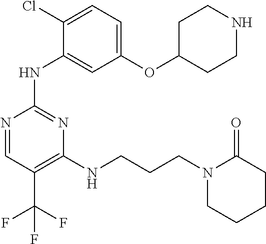 [SMILES: O=C1CCCCN1CCCNc1nc(Nc2cc(OC3CCNCC3)ccc2Cl)ncc1C(F)(F)F]

Intermediate 1 (0.07 g, 0.208 mmol) and tert-butyl 4-(3-amino-4-chloro-phenoxy)piperidine-1-carboxylate (0.075 g, 0.230 mmol) were combined in IPA (3 mL) and heated overnight at 85° C. The reaction mixture was concentrated in vacuo, dissolved in DCM (2 mL) and TFA (0.5 mL) was added. The reaction mixture was stirred for 15 minutes, passed through an SCX cartridge and eluted with 2M NH₃ in MeOH. Purification by preparative HPLC gave the product (0.012 g, 11%). ¹H NMR (CHLOROFORM-d) d: 8.26 (d, J=2.7 Hz, 1H), 8.19 (s, 1H), 7.49 (s, 1H), 7.25 (d, J=8.7 Hz, 1H), 6.84-6.94 (m, 1H), 6.54 (s, 1H), 4.28-4.46 (m, 1H), 3.43-3.59 (m, 4H), 3.25-3.33 (m, 2H), 3.09-3.22 (m, 2H), 2.69-2.83 (m, 2H), 2.39-2.50 (m, 2H), 1.97-2.09 (m, 2H), 1.79-1.87 (m, 6H), 1.70-1.76 (m, 2H). LCMS (m/z): 527.0 [M+H]⁺

### Example 45: 1-[3-[[5-(Trifluoromethyl)-2-[[6-(trifluoromethyl)-1,2,3,4-tetrahydroisoquinolin-7-yl]amino]pyrimidin-4-yl]amino]propyl]piperidin-2-one

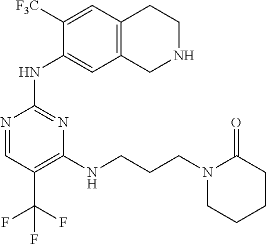 [SMILES: O=C1CCCCN1CCCNc1nc(Nc2cc3c(cc2C(F)(F)F)CCNC3)ncc1C(F)(F)F]

Prepared analogously to Example 19 from Intermediate 1 and 6-(trifluoromethyl)-1,2,3,4-tetrahydroisoquinolin-7-amine. ¹H NMR (CHLOROFORM-d) d: 8.15 (d, J=0.9 Hz, 1H), 7.95 (s, 1H), 7.35 (s, 1H), 7.12 (s, 1H), 6.70-6.88 (m, 1H), 3.97-4.15 (m, 2H), 3.41-3.50 (m, 4H), 3.25-3.33 (m, 2H), 3.14-3.22 (m, 2H), 2.72-2.89 (m, 2H), 2.33-2.53 (m, 2H), 1.76-1.84 (m, 6H); m/z 517 (M+H]⁺

### Example 46: 1-[3-[[2-[(3-Ethoxy-1-methyl-pyrazol-4-yl)amino]-5-(trifluoromethyl)pyrimidin-4-yl]amino]propyl]piperidin-2-one

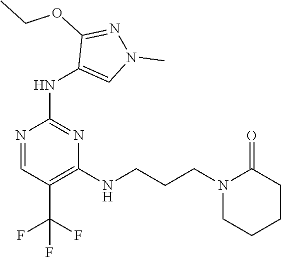 [SMILES: CCOc1nn(C)cc1Nc1ncc(C(F)(F)F)c(NCCCN2CCCCC2=O)n1]

Intermediate 1 (0.050 g, 0.149 mmol), 3-ethoxy-1-methyl-pyrazol-4-amine hydrochloride (0.029 g, 0.164 mmol) and IPA (2 mL) were combined in a microwave vial and heated at 80° C. overnight. The reaction mixture was cooled, dissolved in MeOH and purified via preparative HPLC. The product was passed through an SCX cartridge, to give the product (0.042 g, 64%). ¹H NMR (CHLOROFORM-d) d: 8.04-8.22 (m, 1H), 7.63-7.80 (m, 1H), 6.37-6.92 (m, 2H), 4.22-4.33 (m, 2H), 3.75 (s, 3H), 3.45-3.53 (m, 4H), 3.20-3.37 (m, 2H), 2.37-2.51 (m, 2H), 1.78-1.87 (m, 6H), 1.40 (t, J=7.1 Hz, 3H). LCMS (m/z): 442.0 [M+H]⁺

### Examples 47-52

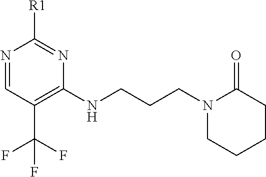

| Example # | R1 | IUPAC name | Analytical data |
|---|---|---|---|
| 47 | 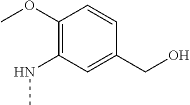 | 1-[3-[[2-[5- (Hydroxymethyl)-2- methoxy-anilino]-5- (trifluoromethyl)pyrimidin- 4- yl]amino]propyl]piperidin- 2-one | ¹H NMR (CHLOROFORM-d) d: 8.57-8.67 (m, 1H), 8.13 (s, 1H), 7.80 (br. s., 1H), 6.94 (dd, J = 8.2, 1.8 Hz, 1H), 6.84 (d, J = 8.2 Hz, 1H), 6.33 (br. s., 1H), 4.66 (s, 2H), 4.05-4.19 (m, 1H), 3.89 (s, 3H), 3.54-3.74 (m, 2H), 3.40-3.53 |
| | | | (m, 2H), 3.20-3.33 (m, 2H), 2.33- |
| | | | 2.47 (m, 2H), 1.66-1.93 (m, 6H); |
| | | | (m/z): 454 [M + H]⁺ |
| 48 | 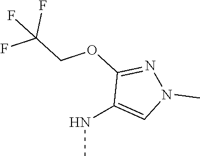 | 1-[3-[[2-[[1-Methyl-3- (2,2,2- trifluoroethoxy)pyrazol-4- yl]amino]-5- (trifluoromethyl)pyrimidin- 4- yl]amino]propyl]piperidin- 2-one | ¹H NMR (CHLOROFORM-d) d: 8.06-8.19 (m, 1H), 7.70-7.85 (m, 1H), 6.35-6.85 (m, 2H), 4.49-4.69 (m, 2H), 3.75 (s, 3H), 3.39-3.57 (m, 4H), 3.21-3.37 (m, 2H), 2.45 (t, J = 6.0 Hz, 2H), 1.73-1.94 (m, 6H); (m/z): 496 [M + H]⁺ |
| 49 | 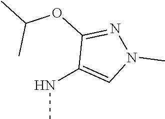 | 1-[3-[[2-[(3-Isopropoxy-1- methyl-pyrazol-4- yl)amino]-5- (trifluoromethyl)pyrimidin- 4- yl]amino]propyl]piperidin- 2-one | ¹H NMR (CHLOROFORM-d) d: 8.03-8.22 (m, 1H), 7.69 (br. s., 1H), 6.82 (br. s., 1H), 6.39-6.66 (m, 1H), 4.73-4.98 (m, 1H), 3.75 (s, 3H), 3.38-3.60 (m, 4H), 3.22- 3.36 (m, 2H), 2.45 (t, J = 6.0 Hz, 2H), 1.72-1.92 (m, 6H), 1.36 (d, J = 6.0 Hz, 6H); (m/z): 456 [M + H]⁺ |
| 50 | 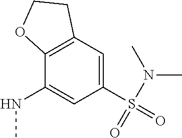 | N,N-dimethyl-7-[[4-[3-(2- oxo-1- piperidyl)propylamino]-5- (trifluoromethyl)pyrimidin- 2-yl]amino]-2,3- dihydrobenzofuran-5- sulfonamide | ¹H NMR (CHLOROFORM-d) d: 8.88 (d, J = 1.4 Hz, 1H), 8.15 (d, J = 0.9 Hz, 1H), 7.30 (br. s., 1H), 7.28 (d, J = 0.9 Hz, 1H), 7.18 (br. s., 1H), 4.77 (t, J = 8.7 Hz, 2H), 3.57-3.64 (m, 2H), 3.44-3.49 (m, 2H), 3.28-3.38 (m, 4H), 2.70 (s, 6H), 2.37-2.48 (m, 2H), 1.80-1.92 (m, 6H); (m/z): 543 [M + H]⁺ |
| 51 | 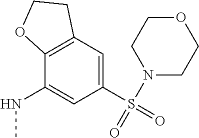 | 1-[3-[[2-[(5- Morpholinosulfonyl-2,3- dihydrobenzofuran-7- yl)amino]-5- (trifluoromethyl)pyrimidin- 4- yl]amino]propyl]piperidin- 2-one | ¹H NMR (CHLOROFORM-d) d: 8.86 (d, J = 1.4 Hz, 1H), 8.16 (s, 1H), 7.31 (br. s., 1H), 7.22-7.25 (m, 1H), 7.19 (br. s., 1H), 4.78 (t, J = 8.9 Hz, 2H), 3.71-3.79 (m, 4H), 3.55-3.63 (m, 2H), 3.43-3.47 (m, 2H), 3.27-3.41 (m, 4H), 2.96-3.05 (m, 4H), 2.37-2.48 (m, 2H), 1.81- 1.92 (m, 6H); (m/z): 585 [M + H]⁺ |

### Example 52: 1-[3-[[2-[(5-Piperazin-1-ylsulfonyl-2,3-dihydrobenzofuran-7-yl)amino]-5-(trifluoromethyl)pyrimidin-4-yl]amino]propyl]piperidin-2-one

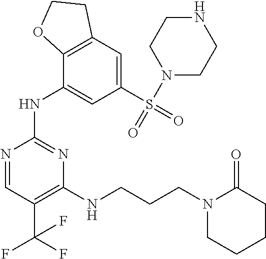 [SMILES: O=C1CCCCN1CCCNc1nc(Nc2cc(S(=O)(=O)N3CCNCC3)cc3c2OCC3)ncc1C(F)(F)F]

Prepared analogously to Example 19 from Intermediate 1 and tert-butyl 4-[(7-amino-2,3-dihydrobenzofuran-5-yl)sulfonyl]piperazine-1-carboxylate. ¹H NMR (CHLOROFORM-d) d: 8.83 (d, J=1.4 Hz, 11H), 8.14 (d, J=0.9 Hz, 11H), 7.46 (br. s, 11H), 7.23 (d, J=1.8 Hz, 11H), 7.10-7.20 (m, 11H), 4.74 (t, J=8.9 Hz, 2H), 3.52-3.63 (m, 2H), 3.41-3.51 (m, 2H), 3.25-3.37 (m, 4H), 2.84-3.04 (m, 8H), 2.38-2.48 (m, 2H), 1.70-1.88 (in, 7H); LCMS (m/z): 584 [M+H]⁺

### Example 53: 1-[3-[[2-[[3-Methoxy-1-(2-morpholinoethyl)pyrazol-4-yl]amino]-5-(trifluoromethyl)pyrimidin-4-yl]amino]propyl]piperidin-2-one

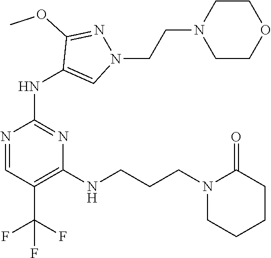 [SMILES: COc1nn(CCN2CCOCC2)cc1Nc1ncc(C(F)(F)F)c(NCCCN2CCCCC2=O)n1]

Intermediate 1 (0.050 g, 0.149 mmol), 3-methoxy-1-(2-morpholinoethyl)pyrazol-4-amine (0.037 g, 0.164 mmol) and IPA (2 mL) were combined and heated at 120° C. in the microwave. The reaction mixture was cooled, concentrated, and purified by preparative HPLC (low pH buffer). The product was passed through an SCX cartridge, giving the final product, (0.046 g, 53% yield). ¹H NMR (CHLOROFORM-d) d: 8.10-8.17 (m, 1H), 7.76 (br. s., 1H), 6.78-6.94 (m, 1H), 6.58 (br. s., 1H), 4.01-4.13 (m, 2H), 3.95 (s, 3H), 3.66-3.73 (m, 4H), 3.40-3.52 (m, 4H), 3.22-3.33 (m, 2H), 2.78 (t, J=6.9 Hz, 2H), 2.40-2.53 (m, 6H), 1.77-1.89 (m, 6H). LCMS (m/z): 527.1 [M+H]⁺.

### Examples 54-55

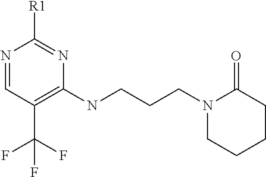

Prepared analogously to Example 53 from Intermediate 1 and the appropriate amine.

| Example # | R1 | IUPAC name | 1H NMR |
|---|---|---|---|
| 54 | 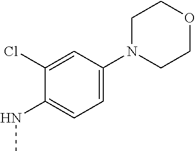 | 1-[3-[[2-(2-Chloro-4- morpholino-anilino)- 5- (trifluoromethyl) pyrimidin-4- yl]amino]propyl] piperidin-2-one | ¹H NMR (CHLOROFORM-d) d: 8.14 (s, 1H), 7.97-8.05 (m, 1H), 7.23 (dd, J = 8.7, 2.3 Hz, 1H), 7.08-7.15 (m, 1H), 7.01 (d, J = 8.7 Hz, 1H), 6.85-6.95 (m, 1H), 3.85- 3.93 (m, 4H), 3.48-3.54 (m, 4H), 3.27- 3.34 (m, 2H), 2.98-3.08 (m, 4H), 2.39- 2.50 (m, 2H), 1.78-1.89 (m, 6H); LCMS (m/z): 513 [M + H]⁺ |
| 55 | 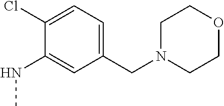 | 1-[3-[[2-[2-Chloro-5- (morpholinomethyl) anilino]-5- (trifluoromethyl) pyrimidin-4- yl]amino]propyl] piperidin-2-one | ¹H NMR (CHLOROFORM-d) d: 8.52 (d, J = 1.8 Hz, 1H), 8.18 (d, J = 0.9 Hz, 1H), 7.56 (br. s, 1H), 7.34 (d, J = 8.2 Hz, 1H), 6.93-7.05 (m, 2H), 3.69-3.77 (m, 4H), 3.43-3.60 (m, 6H), 3.25-3.34 (m, 2H), 2.39-2.64 (m, 6H), 1.73-1.91 (m, 6H). LCMS (m/z): 527 [M + H]⁺ |

### Example 56: 1-[3-[[2-[(2-Acetyl-6-chloro-3,4-dihydro-1H-isoquinolin-7-yl)amino]-5-(trifluoromethyl)pyrimidin-4-yl]amino]propyl]piperidin-2-one

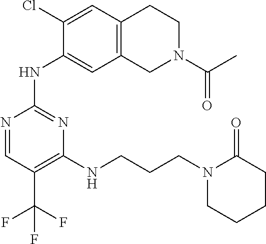 [SMILES: CC(=O)N1CCc2cc(Cl)c(Nc3ncc(C(F)(F)F)c(NCCCN4CCCCC4=O)n3)cc2C1]

To a solution of 1-[3-[[2-[(6-chloro-1,2,3,4-tetrahydroisoquinolin-7-yl)amino]-5-(trifluoromethyl)-pyrimidin-4-yl]amino]propyl]piperidin-2-one (0.030 g, 0.062 mmol) in Et₃N (18 μL, 0.124 mmol) and DCM was added acetyl chloride (5.5 μL, 0.77 mmol) and the reaction mixture left to stir overnight at room temperature. The reaction mixture was concentrated in vacuo and purified via preparative HPLC, yielding the product (0.011 g, 34%). ¹H NMR (CHLOROFORM-d) Shift: 8.37 (s, 1H), 8.16 (s, 1H), 7.53 (s, 1H), 7.17-7.22 (m, 1H), 6.88-7.04 (m, 1H), 4.60-4.74 (m, 2H), 3.65-3.86 (m, 2H), 3.45-3.56 (m, 4H), 3.27-3.36 (m, 2H), 2.74-2.92 (m, 2H), 2.41-2.50 (m, 2H), 2.16-2.35 (m, 3H), 1.78-1.89 (m, 6H). LCMS (m/z): 525.0 [M+H]⁺.

### Example 57: 1-[3-[[2-(2-Chloro-5-hydroxy-anilino)-5-(trifluoromethyl)pyrimidin-4-yl]amino]propyl]piperidin-2-one

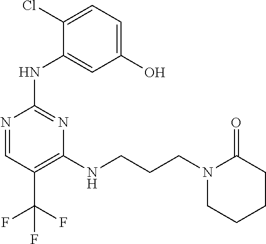 [SMILES: O=C1CCCCN1CCCNc1nc(Nc2cc(O)ccc2Cl)ncc1C(F)(F)F]

### Step 1: Tert-butyl-(4-chloro-3-nitro-phenoxy)-dimethyl-silane

To a solution of 4-chloro-3-nitrophenol (0.500 g, 0.289 mmol) and Et₃N (0.843 g, 0.578 mmol) in DCM (20 mL) was added tert-butyldimethylsilyl chloride (0.528 g, 0.318 mmol) and the reaction mixture was left to stir for 1 h at room temperature. The reaction mixture was diluted with DCM and water, the organic layer separated, dried and concentrated directly onto SiO₂. Purified via column chromatography (0-10% EtOAc in PE) to give the desired product, tert-butyl-(4-chloro-3-nitro-phenoxy)-dimethyl-silane (0.637 g, 73% yield) as a pale-yellow oil. ¹H NMR (CHLOROFORM-d) d: 7.39 (d, J=8.7 Hz, 1H), 7.33 (d, J=2.7 Hz, 1H), 6.99 (dd, J=8.7, 2.7 Hz, 1H), 0.99 (s, 9H), 0.24 (s, 6H).

### Step 2: 5-[Tert-butyl(dimethyl)silyl]oxy-2-chloro-aniline

To a solution of tert-butyl-(4-chloro-3-nitro-phenoxy)-dimethyl-silane (0.637 g, 2.10 mmol) in EtOAc (30 mL) was added SnCl₂·2H₂O (2.37 g, 10.5 mmol) and the reaction mixture left to stir overnight. The reaction mixture was poured in saturated NaHCO₃ (aq), the organic layer separated, dried and concentrated in vacuo to give the product, 5-[tert-butyl(dimethyl)silyl]oxy-2-chloro-aniline (536 mg, 94%). ¹H NMR (CHLOROFORM-d) d: 7.07 (d, J=8.7 Hz, 1H), 6.30 (d, J=2.7 Hz, 1H), 6.21 (dd, J=8.7, 2.7 Hz, 1H), 0.97 (s, 9H), 0.18 (s, 6H).

### Step 3: 1-[3-[[2-[5-[Tert-butyl(dimethyl)silyl]oxy-2-chloro-anilino]-5-(trifluoromethyl)pyrimidin-4-yl]amino]propyl]piperidin-2-one

Intermediate 1 (0.100 g, 0.298 mmol), 5-[tert-butyl(dimethyl)silyl]oxy-2-chloro-aniline (0.089 g, 0.327 mmol) and IPA (2 mL) were combined in a microwave vial and heated at 80° C. overnight. The reaction mixture was cooled and concentrated in vacuo onto silica. The compound was purified via column chromatography (10-100% EtOAc in PE) to give a yellow oil (0.114 g, 65%), as a yellow oil. ¹H NMR (CHLOROFORM-d) Shift: 8.16-8.19 (m, 1H), 8.14 (s, 1H), 7.43-7.59 (m, 1H), 7.20 (d, J=8.7 Hz, 1H), 6.86-6.98 (m, 1H), 6.46 (dd, J=8.7, 2.7 Hz, 1H), 3.45-3.55 (m, 4H), 3.25-3.30 (m, 2H), 2.40-2.51 (m, 2H), 1.79-1.86 (m, 6H), 0.99 (s, 9H), 0.22 (s, 6H). LCMS (m/z): 558.0 [M+H]⁺.

### Step 4: 1-[3-[[2-(2-Chloro-5-hydroxy-anilino)-5-(trifluoromethyl)pyrimidin-4-yl]amino]propyl]piperidin-2-one

1-[3-[[2-[5-[Tert-butyl(dimethyl)silyl]oxy-2-chloro-anilino]-5-(trifluoromethyl)pyrimidin-4-yl]amino]-propyl]piperidin-2-one (0.114 g, 0.205 mmol) in THF (5 mL) was added TBAF (1M in THF, 0.31 mL, 0.307 mmol) and the reaction mixture stirred at room temperature for 1 hour. The reaction mixture was concentrated in vacuo onto silica and purified via column chromatography to give a white solid (0.090 g, 99%). ¹H NMR (CHLOROFORM-d) Shift: 10.10 (br. s., 1H), 8.02 (s, 1H), 7.42 (d, J=2.7 Hz, 1H), 7.19 (d, J=8.7 Hz, 1H), 7.03-7.12 (m, 1H), 6.46-6.74 (m, 1H), 3.43-3.53 (m, 4H), 3.30-3.37 (m, 2H), 2.39-2.52 (m, 2H), 1.80-1.89 (m, 6H). LCMS (m/z): 443.9 [M+H]⁺.

### Example 58: 1-[3-[[2-[2-Chloro-5-[2-(dimethylamino)ethoxy]anilino]-5-(trifluoromethyl)pyrimidin-4-yl]amino]propyl]piperidin-2-one

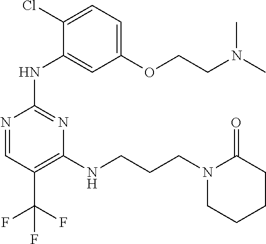 [SMILES: CN(C)CCOc1ccc(Cl)c(Nc2ncc(C(F)(F)F)c(NCCCN3CCCCC3=O)n2)c1]

To a mixture of 1-[3-[[2-(2-chloro-5-hydroxy-anilino)-5-(trifluoromethyl)pyrimidin-4-yl]amino]propyl]piperidin-2-one (0.030 g, 0.068 mmol) and K₂CO₃ (0.019 g, 0.135 mmol) in DMF (1 mL) was added 2-chloro-N,N-dimethyl-ethanamine hydrochloride (0.011 g, 0.080 mmol). The reaction mixture was heated at 70° C. overnight, concentrated in vacuo and purified via preparative HPLC to give the product (3 mg, 9%). ¹H NMR (CHLOROFORM-d) d: 8.25 (d, J=2.7 Hz, 1H), 8.19 (d, J=0.9 Hz, 1H), 7.49 (s, 1H), 7.25 (d, J=8.7 Hz, 1H), 6.85-6.93 (m, 1H), 6.55 (dd, J=8.7, 2.7 Hz, 1H), 4.05-4.14 (m, 2H), 3.44-3.57 (m, 4H), 3.25-3.33 (m, 2H), 2.74-2.83 (m, 2H), 2.41-2.50 (m, 2H), 2.38 (s, 6H), 1.77-1.86 (m, 6H). LCMS (m/z): 515.0 [M+H]⁺.

### Example 59: 1-[3-[[2-[2-Chloro-5-(2-morpholinoethoxy)anilino]-5-(trifluoromethyl)pyrimidin-4-yl]amino]propyl]piperidin-2-one

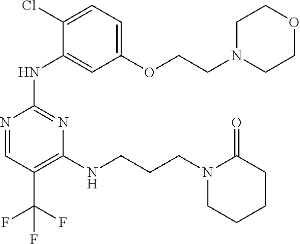 [SMILES: O=C1CCCCN1CCCNc1nc(Nc2cc(OCCN3CCOCC3)ccc2Cl)ncc1C(F)(F)F]

Prepared analogously to Example 58 from 1-[3-[[2-(2-chloro-5-hydroxy-anilino)-5-(trifluoromethyl)-pyrimidin-4-yl]amino]propyl]piperidin-2-one and 2-morpholinoethanamine hydrochloride to give the product (0.010 g, 27%). ¹H NMR (CHLOROFORM-d) d: 8.24 (d, J=3.2 Hz, 1H), 8.19 (d, J=0.9 Hz, 1H), 7.49 (s, 1H), 7.26 (d, J=8.7 Hz, 1H), 6.86-7.01 (m, 1H), 6.47-6.58 (m, 1H), 4.12 (t, J=5.7 Hz, 2H), 3.70-3.77 (m, 4H), 3.44-3.57 (m, 4H), 3.26-3.33 (m, 2H), 2.82 (t, J=5.7 Hz, 2H), 2.56-2.63 (m, 4H), 2.41-2.50 (m, 2H), 1.78-1.87 (m, 6H). LCMS (m/z): 557.0 [M+H]⁺.

### Example 60: 1-[3-[[2-[2-Methoxy-5-(methylaminomethyl)anilino]-5-(trifluoromethyl)pyrimidin-4-yl]amino]propyl]piperidin-2-one

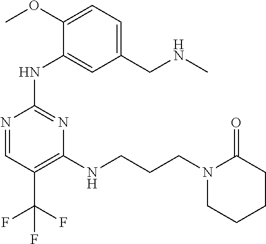 [SMILES: CNCc1ccc(OC)c(Nc2ncc(C(F)(F)F)c(NCCCN3CCCCC3=O)n2)c1]

### Step 1: 4-Methoxy-3-[[4-[3-(2-oxo-1-piperidyl)propylamino]-5-(trifluoromethyl)pyrimidin-2-yl]amino]benzaldehyde

To a solution of 1-[3-[[2-[5-(hydroxymethyl)-2-methoxy-anilino]-5-(trifluoromethyl)pyrimidin-4-yl]amino]propyl]piperidin-2-one (0.140 g, 0.309 mmol) in THF (10 mL) was added Dess-Martin periodinane (0.087 g, 0.464 mmol) and the reaction mixture left to stir for 2 hours at room temperature. The reaction mixture was concentrated in vacuo onto silica and purified via column chromatography (40-80% EtOAc in PE) to give a light brown solid (0.083 g, 60% Yield). ¹H NMR (CHLOROFORM-d) d: 9.90 (s, 1H), 9.18 (d, J=2.3 Hz, 1H), 8.17 (d, J=0.9 Hz, 1H), 7.77 (s, 1H), 7.51 (dd, J=8.2, 2.3 Hz, 1H), 7.03-7.13 (m, 1H), 7.00 (d, J=8.2 Hz, 1H), 4.00 (s, 3H), 3.59-3.69 (m, 2H), 3.45-3.55 (m, 2H), 3.24-3.36 (m, 2H), 2.41-2.52 (m, 2H), 1.90-1.97 (m, 2H), 1.80-1.86 (m, 4H). LCMS (m/z): 452.0 [M+H]⁺.

### Step 2: 1-[3-[[2-[2-Methoxy-5-(methylaminomethyl)anilino]-5-(trifluoromethyl)pyrimidin-4-yl]amino]propyl]piperidin-2-one

To a solution of 4-methoxy-3-[[4-[3-(2-oxo-1-piperidyl)propylamino]-5-(trifluoromethyl)pyrimidin-2-yl]amino]benzaldehyde (0.027 g, 0.060 mmol) in DCM/MeOH (5 mL) was added methylamine (0.7 μL, 0.072 mmol), followed by Na(OAc)₃BH (0.019 g, 0.090 mmol) and the reaction mixture left to stir overnight at room temperature. The reaction mixture was concentrated in vacuo, taken up in MeOH and submitted for preparative HPLC (pH 1), after which it was passed through an SCX cartridge, giving the desired product (7 mg, 20%). ¹H NMR (CHLOROFORM-d) δ: 8.48 (d, J=2.3 Hz, 1H), 8.16 (s, 1H), 7.68 (s, 1H), 6.95 (dd, J=8.2, 2.3 Hz, 1H), 6.76-6.86 (m, 2H), 3.88 (s, 3H), 3.72-3.76 (m, 2H), 3.53-3.60 (m, 2H), 3.49-3.50 (m, 1H), 3.45-3.47 (m, 2H), 3.24-3.33 (m, 2H), 2.43-2.47 (m, 5H), 1.78-1.86 (m, 6H). LCMS (m/z): 467.1 [M+H]⁺.

### Example 61: 1-[3-[[2-[2-Methoxy-5-(morpholinomethyl)anilino]-5-(trifluoromethyl)pyrimidin-4-yl]amino]propyl]piperidin-2-one

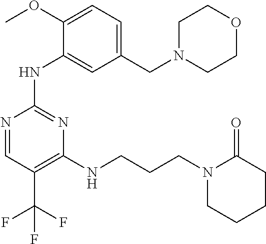 [SMILES: COc1ccc(CN2CCOCC2)cc1Nc1ncc(C(F)(F)F)c(NCCCN2CCCCC2=O)n1]

Prepared analogously to Example 60 using morpholine in Step 2. ¹H NMR (CHLOROFORM-d) Shift: 8.49 (d, J=1.8 Hz, 1H), 8.16 (s, 1H), 7.67-7.74 (m, 1H), 6.97 (dd, J=8.2, 1.8 Hz, 1H), 6.82-6.88 (m, 2H), 3.90 (s, 3H), 3.71-3.79 (m, 4H), 3.46-3.68 (m, 6H), 3.26-3.34 (m, 2H), 2.40-2.75 (m, 6H), 1.78-1.91 (m, 6H); LCMS (m/z): 523 [M+H]⁺.

### Example 62: N-(3-((2-((1,2,3,4-tetrahydroisoquinolin-6-yl)amino)-5-(trifluoromethyl)pyrimidin-4-yl)amino)propyl)cyclobutanecarboxamide

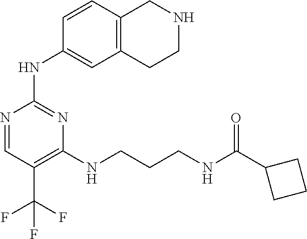 [SMILES: O=C(NCCCNc1nc(Nc2ccc3c(c2)CCNC3)ncc1C(F)(F)F)C1CCC1]

### Step 1: Tert-butyl 6-((4-chloro-5-(trifluoromethyl)pyrimidin-2-yl)amino)-3,4-dihydroisoquinoline-2(1H)-carboxylate

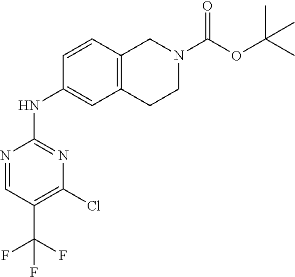 [SMILES: CC(C)(C)OC(=O)N1CCc2cc(Nc3ncc(C(F)(F)F)c(Cl)n3)ccc2C1]

Zinc chloride (3.04 mL, 1M in Et₂O, 3.04 mmol) was added dropwise to a stirring solution of 2,4-dichloro-5-trifluoromethylpyrimidine (0.300 g, 1.38 mmol) in 1:1 DCE:^{t}BuOH (12 mL) at 0° C. The reaction mixture was stirred for 1 hour before being treated with 6-amino-2-N-1,2,3,4-tetrahydroisoquinoline (341 mg, 1.38 mmol) followed by a solution of triethylamine (0.21 mL, 1.52 mmol) in 1:1 DCE:^{t}BuOH (4 mL). The mixture was stirred for 2 hours 30 mins before being concentrated in vacuo, then taken up in DCM and concentrated onto silica in vacuo. The crude reaction mixture was purified by column chromatography (10 g column, 60-70% EA in PE) to give a white solid (370 mg, 75%); ¹H NMR (400 MHz, CHLOROFORM-d) δ ppm 8.59 (s, 1H), 7.36 (m, 2H), 7.11 (d, J=8.24 Hz, 1H), 4.55 (s, 2H), 3.65 (t, J=5.04 Hz, 2H), 2.84 (t, J=5.50 Hz, 2H), 1.48 (s, 9H).

### Step 2: N-(3-((2-((1,2,3,4-tetrahydroisoquinolin-6-yl)amino)-5-(trifluoromethyl)pyrimidin-4-yl)amino)propyl)cyclobutanecarboxamide

N-(3-aminopropyl)cyclobutanecarboxamide (12 mg, 0.079 mmol) and triethylamine (0.02 mL, 0.119 mmol) was added to a suspension of tert-butyl 6-((4-chloro-5-(trifluoromethyl)pyrimidin-2-yl)amino)-3,4-dihydroisoquinoline-2(1H)-carboxylate (34 mg, 0.079 mmol) in IPA (2 mL). The reaction mixture was stirred at 70° C. for 2 hours then cooled to room temperature and concentrated in vacuo. This was taken up in DCM (5 mL), TFA (1 mL) added and the reaction mixture stirred at room temperature for 1 h. The mixture was concentrated and the resulting residue purified by preparative HPLC (pH1), and then desalted (SCX cartridge). The solvent was concentrated in vacuo to give a white solid (2.1 mg, 6%); ¹H NMR (400 MHz, DMSO-d₆) δ ppm 9.45 (s, 1H), 8.12 (s, 1H), 7.63 (t, J=5.50 Hz, 1H), 7.51 (s, 1H), 7.36 (dd, J=9.62, 2.29 Hz, 1H), 7.12 (t, J=4.58 Hz, 1H), 6.88 (d, J=8.70 Hz, 1H), 3.77 (s, 2H), 3.40 (q, J=6.00 Hz, 2H), 3.04 (m, 2H), 2.92 (m, 2H), 2.63 (t, J=5.50 Hz, 2H), 2.07 (m, 2H), 1.95 (m, 2H), 1.82 (m, 1H), 1.67 (m, 4H); LCMS (m/z): 449.1 [M+H]⁺.

### Examples 63-67

Prepared analogously to Example 62 from tert-butyl 6-((4-chloro-5-(trifluoromethyl)pyrimidin-2-yl)amino)-3,4-dihydroisoquinoline-2(1H)-carboxylate and the appropriate amine in step 1.

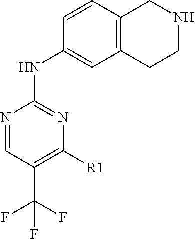

| Ex. | R1 | Name | Analytical data |
|---|---|---|---|
| 63 | 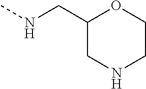 | N4-(morpholin-2-ylmethyl)- N2-(1,2,3,4- tetrahydroisoquinolin-6-yl)- 5- (trifluoromethyl)pyrimidine- 2,4-diamine | ¹H NMR (CHLOROFORM-d) d: 8.15 (d, J = 0.9 Hz, 1H), 7.34 (d, J = 1.8 Hz, 1H), 7.28-7.32 (m, 1H), 7.22-7.26 (m, 1H), 6.97 (d, J = 8.2 Hz, 1H), 5.60 (br. s., 1H), 3.99 (s, 2H), 3.88-3.95 (m, 1H), 3.59-3.76 (m, 3H), 3.38-3.47 (m, 1H), 3.14 (t, J = 6.0 |
| | | | Hz, 2H), 2.76-2.96 (m, 5H), 2.60-2.70 |
| | | | (m, 1H); LCMS (m/z): 409.1 [M + H]⁺. |
| 64 | 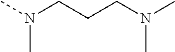 | N4-[3- (dimethylamino)propyl]- N4-methyl-N2-(1,2,3,4- tetrahydroisoquinolin-6-yl)- | ¹H NMR (400 MHz, CHLOROFORM-d) δ ppm 8.28 (s, 1H), 7.43 (br. s, NH), 7.30 (dd, J = 8.24, 1.83 Hz, 1H), 7.26 (s, 1H), 6.94 (d, J = 8.24 Hz, 1H), 3.97 (s, 2H), |
| | | 5- | 3.61 (t, J = 7.33 Hz, 2H), 3.11 (m, J = 1.37 |
| | | (trifluoromethyl)pyrimidine- | Hz, 5H), 2.77 (t, J = 5.95 Hz, 2H), 2.24 |
| | | 2,4-diamine | (t, J = 6.87 Hz, 2H), 2.18 (s, 6H), 1.79 |
| | | | (quin, J = 7.33 Hz, 2H); LCMS (m/z): |
| | | | 409.2 [M + H]⁺. |
| 65 | 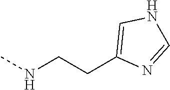 | N4-[2-(1H-imidazol-5- yl)ethyl]-N2-(1,2,3,4- tetrahydroisoquinolin-6-yl)- 5-(trifluoromethyl)pyrimidine- 2,4-diamine | ¹H NMR (400 MHz, DMSO-d₆) δ ppm 9.61 (br. s, 1H), 8.15 (s, 1H), 7.65 (s, 1H), 7.55 (s, 1H), 7.50 (dd, J = 9.16, 1.83 Hz, 1H), 7.31 (t, J = 5.95 Hz, 1H), 7.04 (d, J = 8.70 Hz, 1H), 6.79 (s, 1H), 4.14 (s, 2H), 3.66 (q, J = 6.87 Hz, 2H), 3.26 (t, |
| | | | J = 6.00 Hz, 2H), 2.80 (m, 4H); |
| | | | LCMS (m/z): 404.1 [M + H]⁺. |
| 66 | 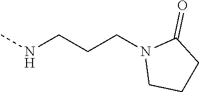 | 1-[3-[[2-(1,2,3,4- tetrahydroisoquinolin-6- ylamino)-5- (trifluoromethyl)pyrimidin- 4-yl]amino]propyl] pyrrolidin-2-one | ¹H NMR (400 MHz, CHLOROFORM-d) δ ppm 8.11 (s, 1H), 7.39 (s, 1H), 7.34 (br. s, 1H), 7.28 (dd, J = 7.33, 2.29 Hz, 1H), 6.94 (d, J = 8.24 Hz, 1H), 6.51 (t, J = 5.50 Hz, 1H), 3.98 (s, 2H), 3.50 (dt, J = 9.16, 5.95 Hz, 2H), 3.40 (t, J = 6.87 Hz, 2H), |
| | | | 3.35 (t, J = 6.40 Hz, 2H), 3.13 (t, J = 5.95 |
| | | | Hz, 2H), 2.79 (t, J = 5.95 Hz, 2H), 2.43 (t, |
| | | | J = 7.33 Hz, 2H), 2.04 (quin, J = 7.33 Hz, |
| | | | 2H), 1.79 (quin, J = 6.07 Hz, 2H); |
| | | | LCMS (m/z): 435.1 [M + H]⁺. |
| 67 | 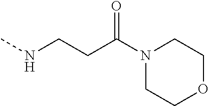 | 1-Morpholino-3-[[2- (1,2,3,4- tetrahydroisoquinolin-6- ylamino)-5- (trifluoromethyl)pyrimidin- 4-yl]amino]propan-1-one | ¹H NMR (400 MHz, CHLOROFORM-d) δ ppm 8.12 (s, 1H), 7.33 (s, 1H), 7.31 (dd, J = 8.24, 2.29 Hz, 1H), 6.95 (d, J = 8.24 Hz, 1H), 6.09 (t, J = 5.50 Hz, 1H), 3.98 (s, 2H), 3.86 (dt, J = 6.87, 5.50 Hz, 2H), 3.62 (m, 8H), 3.36 (t, J = 5.04 Hz, 2H), 3.13 (t, J = 5.95 Hz, 2H), 2.78 (t, |
| | | | J = 5.95 Hz, 2H), 2.61 (t, J = 5.95 Hz, 2H); |
| | | | LCMS (m/z): 451.1 [M + H]⁺. |

### Example 68: 1-[4-[[2-[(6-Methoxy-1,2,3,4-tetrahydroisoquinolin-7-yl)amino]-5-(trifluoromethyl)pyrimidin-4-yl]amino]-1-piperidyl]ethanone

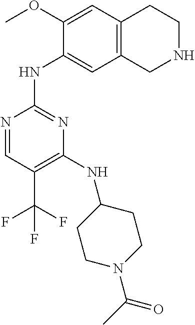 [SMILES: COc1cc2c(cc1Nc1ncc(C(F)(F)F)c(NC3CCN(C(C)=O)CC3)n1)CNCC2]

### Step 1: Tert-butyl 7-[[4-chloro-5-(trifluoromethyl)pyrimidin-2-yl]amino]-6-methoxy-3,4-dihydro-1H-isoquinoline-2-carboxylate

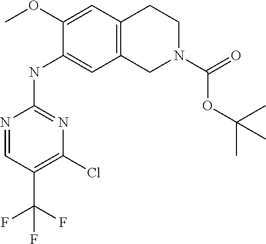 [SMILES: COc1cc2c(cc1Nc1ncc(C(F)(F)F)c(Cl)n1)CN(C(=O)OC(C)(C)C)CC2]

To a solution of 2,4-dichloro-5-(trifluoromethyl)pyrimidine (0.390 g, 1.79 mmol) in DCE (15 mL) and ^{t}BuOH (15 mL), at 0° C., was added ZnCl₂ (1 M in Et₂O) (3.96 mL, 3.96 mmol) dropwise and left to stir for 1 hour. A solution of tert-butyl 7-amino-6-methoxy-3,4-dihydro-1H-isoquinoline-2-carboxylate (0.500 g, 1.79 mmol) and Et₃N (0.52 mL, 3.58 mmol) in DCE and ^{t}BuOH was then added dropwise and the reaction mixture left to stir and warm over the weekend. The reaction mixture was concentrated in vacuo, taken up in DCM and absorbed onto silica. The compound was purified via column chromatography (0-15% EtOAc in PE), the fractions containing product combined and concentrated in vacuo to give the product, tert-butyl 7-[[4-chloro-5-(trifluoromethyl)pyrimidin-2-yl]amino]-6-methoxy-3,4-dihydro-1H-isoquinoline-2-carboxylate (0.760 g, 1.66 mmol, 92% Yield), as a colorless oil that solidified upon standing. ¹H NMR (400 MHz, CHLOROFORM-d) d 8.57-8.63 (m, 1H), 8.10-8.14 (m, 1H), 6.66-6.69 (m, 1H), 4.54-4.59 (m, 2H), 3.89-3.90 (m, 3H), 3.63-3.69 (m, 2H), 2.78-2.85 (m, 2H), 1.50-1.52 (m, 9H); LCMS (m/z): 358.9 [M-CO₂^{t}Bu+H].⁺

### Step 2: 1-[4-[[2-[(6-Methoxy-1,2,3,4-tetrahydroisoquinolin-7-yl)amino]-5-(trifluoromethyl)pyrimidin-4-yl]amino]-1-piperidyl]ethenone

Tert-butyl 7-[[4-chloro-5-(trifluoromethyl)pyrimidin-2-yl]amino]-6-methoxy-3,4-dihydro-1H-isoquinoline-2-carboxylate (0.045 g, 0.098 mmol), 1-(4-amino-1-piperidyl)ethanone (0.015 g, 0.108 mmol) and IPA (2 mL) were combined in a microwave vial and heated overnight at 80° C. The reaction mixture was concentrated and TFA added (1 mL), after which the residue was taken up in MeOH and submitted for preparative HPLC (pH 10), yielding the product (9 mg, 20%). ¹H NMR (400 MHz, DMSO-d6) d 8.16 (s, 1H), 8.02 (s, 1H), 7.69 (s, 1H), 6.73 (s, 1H), 6.55-6.64 (m, 1H), 4.38-4.46 (m, 1H), 4.23-4.34 (m, 1H), 3.84-3.90 (m, 1H), 3.80 (s, 5H), 3.00-3.08 (m, 1H), 2.87-2.95 (m, 2H), 2.60-2.68 (m, 2H), 2.51-2.57 (m, 1H), 1.98-2.03 (m, 3H), 1.75-1.86 (m, 2H), 1.56-1.68 (m, 1H), 1.43-1.56 (m, 1H); LCMS (m/z): 465.0 [M+H]⁺.

### Examples 69-73

Prepared analogously to Example 68 from Tert-butyl 7-[[4-chloro-5-(trifluoromethyl)pyrimidin-2-yl]amino]-6-methoxy-3,4-dihydro-1H-isoquinoline-2-carboxylate and the appropriate amine in step 2.

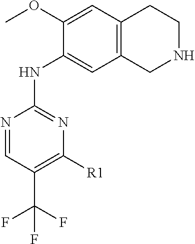

| Ex # | R1 | Name | Analytical data |
|---|---|---|---|
| 1 | 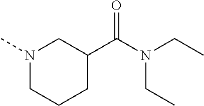 | N,N-diethyl-1-[2-[(6- methoxy-1,2,3,4- tetrahydroisoquinolin-7- yl)amino]-5- (trifluoromethyl)pyrimidin- 4-yl]piperidine-3- carboxamide | ¹H NMR (400 MHz, DMSO-d6) d 8.35 (s, 1H), 8.12-8.18 (m, 1H), 7.57 (s, 1H), 6.71 (s, 1H), 4.08-4.19 (m, 1H), 3.94-4.04 (m, 1H), 3.78 (s, 3H), 3.71 (s, 2H), 3.00-3.25 (m, 6H), 2.85-2.95 (m, 2H), 2.61-2.73 (m, 3H), 1.52-1.82 (m, 4H), 0.99 (t, J = 7.10 Hz, 6H); LCMS (m/z): 507 [M + H]⁺. |
| 2 | 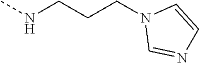 | N4-(3-imidazol-1- ylpropyl)-N2-(6- methoxy-1,2,3,4- tetrahydroisoquinolin-7- yl)-5- | ¹H NMR (400 MHz, DMSO-d6) d 8.13- 8.17 (m, 1H), 7.97-8.01 (m, 1H), 7.67-7.71 (m, 1H), 7.59-7.62 (m, 1H), 7.19-7.25 (m, 1H), 7.15 (s, 1H), 6.86-6.88 (m, 1H), 6.72 (s, 1H), 3.91-3.97 (m, 2H), 3.75-3.81 (m, |
| | | (trifluoromethyl)pyrimidine- | 5H), 3.37-3.40 (m, 2H), 2.90-2.97 (m, 2H), |
| | | 2,4-diamine | 2.62-2.69 (m, 2H), 1.95-2.04 (m, 2H); |
| | | | LCMS (m/z): 448 [M + H]⁺. |
| 3 | 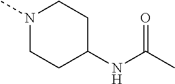 | N-[1-[2-[(6-methoxy- 1,2,3,4- tetrahydroisoquinolin-7- yl)amino]-5- (trifluoromethyl)pyrimidin- 4-yl]-4- | ¹H NMR (400 MHz, DMSO-d6) d 8.35 (s, 1H), 8.20 (s, 1H), 7.81-7.88 (m, 1H), 7.63- 7.71 (m, 1H), 6.74 (s, 1H), 3.93-4.01 (m, 2H), 3.82-3.91 (m, 1H), 3.79 (s, 5H), 3.10-3.19 (m, 2H), 2.93-2.99 (m, 2H), 2.64- 2.71 (m, 2H), 1.74-1.86 (m, 5H), 1.32-1.49 |
| | | piperidyl]acetamide | (m, 2H); LCMS (m/z): 465.0 [M + H]⁺. |
| 4 | 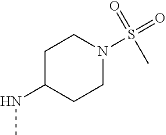 | N2-(6-methoxy-1,2,3,4- tetrahydroisoquinolin-7- yl)-N4-(1- methylsulfonyl-4- piperidyl)-5- (trifluoromethyl)pyrimidine- 2,4-diamine | ¹H NMR (400 MHz, DMSO-d6) d 8.11- 8.18 (m, 1H), 7.96-8.03 (m, 1H), 7.57-7.65 (m, 1H), 6.63-6.73 (m, 2H), 4.06-4.18 (m, 1H), 3.70-3.80 (m, 5H), 3.53-3.64 (m, 2H), 2.84-2.93 (m, 5H), 2.65-2.76 (m, 2H), 2.62 (t, J = 5.50 Hz, 2H), 1.79-1.87 (m, 2H), 1.64-1.76 (m, 2H). |
| 5 | 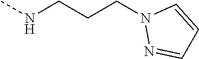 | N2-(6-methoxy-1,2,3,4- tetrahydroisoquinolin-7- yl)-N4-(3-pyrazol-1- ylpropyl)-5- | ¹H NMR (400 MHz, DMSO-d6) d 8.13- 8.17 (m, 1H), 7.99 (s, 1H), 7.75 (s, 1H), 7.70-7.73 (m, 1H), 7.40-7.45 (m, 1H), 7.17- 7.27 (m, 1H), 6.76 (s, 1H), 6.19-6.24 |
| | | (trifluoromethyl)pyrimidine- | (m, 1H), 4.11 (t, J = 7.10 Hz, 2H), 3.83-3.89 |
| | | 2,4-diamine | (m, 2H), 3.80 (s, 3H), 3.39-3.42 (m, 2H), |
| | | | 2.98-3.06 (m, 2H), 2.69-2.75 (m, 2H), 2.05 |
| | | | (t, J = 6.87 Hz, 2H); LCMS (m/z): 448 |
| | | | [M + H]⁺. |

### Example 74: N-[3-[[5-cyclopropyl-2-[(6-methoxy-1,2,3,4-tetrahydroisoquinolin-7-yl)amino]pyrimidin-4-yl]amino]propyl]cyclobutanecarboxamide

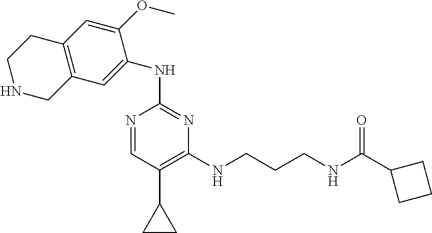 [SMILES: COc1cc2c(cc1Nc1ncc(C3CC3)c(NCCCNC(=O)C3CCC3)n1)CNCC2]

### Step 1: N-[3-[(2-chloro-5-cyclopropyl-pyrimidin-4-yl)amino]propyl]cyclobutanecarboxamide

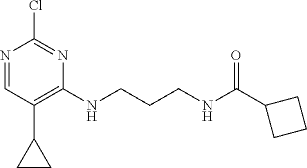 [SMILES: O=C(NCCCNc1nc(Cl)ncc1C1CC1)C1CCC1]

2,4-dichloro-5-cyclopropyl-pyrimidine (0.294 g, 1.08 mmol), N-(3-aminopropyl)cyclobutanecarboxamide (0.170 g, 0.899 mmol), DIPEA (0.33 mL, 1.70 mmol) and IPA (10 mL) were combined and heated at 50° C. for 3 hours. The reaction mixture was cooled and concentrated onto silica and the compound purified via column chromatography (10-100% EtOAc in PE). The fractions were combined and concentrated to give a yellow oil (0.037 g, 13%); LCMS (m/z): 309.1 [M+H]⁺.

### Step 2: N-[3-[[5-cyclopropyl-2-[(6-methoxy-1,2,3,4-tetrahydroisoquinolin-7-yl)amino]pyrimidin-4-yl]amino]propyl]cyclobutanecarboxamide

N-[3-[(2-chloro-5-cyclopropyl-pyrimidin-4-yl)amino]propyl]cyclobutanecarboxamide (0.037 g, 0.120 mmol), tert-butyl 7-amino-6-methoxy-3,4-dihydro-1H-isoquinoline-2-carboxylate (0.037 g, 0.132 mmol) and IPA (2 mL) were combined in a microwave vial and heated overnight at 80° C. The reaction mixture was concentrated, dissolved in DCM (2 mL) and TFA added (0.5 mL), after which the residue was passed through an SCX cartridge and the product eluted with 2M NH₃ in MeOH. The eluent was concentrated, taken up in MeOH and purified by preparative HPLC (pH 10), yielding a white solid (0.013 g, 23% Yield). ¹H NMR (400 MHz, CHLOROFORM-d) d 8.20 (s, 1H), 7.74 (s, 1H), 7.34 (br. s, 1H), 6.57 (s, 1H), 5.90-5.96 (m, 1H), 5.82-5.89 (m, 1H), 3.98 (s, 2H), 3.83-3.89 (m, 3H), 3.57-3.64 (m, 2H), 3.29-3.35 (m, 2H), 3.11-3.19 (m, 2H), 2.80-2.92 (m, 1H), 2.72-2.78 (m, 2H), 2.21-2.30 (m, 2H), 2.02-2.12 (m, 2H), 1.74-1.97 (m, 4H), 1.42-1.52 (m, 1H), 0.88-0.96 (m, 2H), 0.47-0.54 (m, 2H). LCMS (m/z): 451.1 [M+H]⁺.

### Example 75: 1-[3-[[5-Cyclopropyl-2-[(6-methoxy-1,2,3,4-tetrahydroisoquinolin-7-yl)amino]pyrimidin-4-yl]amino]propyl]piperidin-2-one

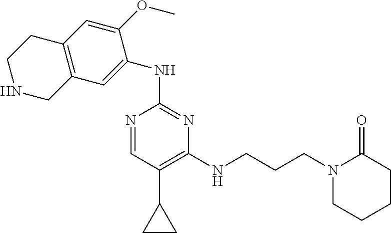 [SMILES: COc1cc2c(cc1Nc1ncc(C3CC3)c(NCCCN3CCCCC3=O)n1)CNCC2]

Prepared analogously to Example 74 except 1-(3-aminopropyl)piperidin-2-one was used in step 2.

¹H NMR (400 MHz, CHLOROFORM-d) d 8.26 (s, 1H), 7.69 (s, 1H), 7.49 (br. s, 1H), 6.52-6.59 (m, 2H), 4.07 (s, 2H), 3.86 (s, 3H), 3.45-3.56 (m, 5H), 3.28-3.34 (m, 2H), 3.21-3.26 (m, 2H), 2.79-2.86 (m, 2H), 2.41-2.44 (m, 2H), 1.79-1.87 (m, 6H), 1.48-1.56 (m, 1H), 0.90-0.97 (m, 2H), 0.46-0.56 (m, 2H).

### Example 76: 1-[3-[[2-[(2-Methyl-3,4-dihydro-1H-isoquinolin-6-yl)amino]-5-(trifluoromethyl)pyrimidin-4-yl]amino]propyl]pyrrolidin-2-one

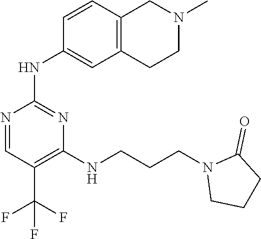 [SMILES: CN1CCc2cc(Nc3ncc(C(F)(F)F)c(NCCCN4CCCC4=O)n3)ccc2C1]

1-[3-[[2-Chloro-5-(trifluoromethyl)pyrimidin-4-yl]amino]propyl]pyrrolidin-2-one (0.045 g, 0.140 mmol), 2-methyl-3,4-dihydro-1H-isoquinolin-6-amine (0.027 g, 0.168 mmol) and IPA (2 mL) were combined and sealed in a microwave vial and heated at 80° C. for 18 hours. The reaction mixture was cooled, concentrated and purified by HPLC to give the product (9 mg, 14%). ¹H NMR (400 MHz, CHLOROFORM-d) d 8.12 (s, 1H), 7.37-7.45 (m, 1H), 7.25-7.29 (m, 1H), 7.06-7.15 (m, 1H), 6.92-6.98 (m, 1H), 6.44-6.55 (m, 1H), 3.46-3.55 (m, 4H), 3.32-3.43 (m, 4H), 2.85-2.94 (m, 2H), 2.64-2.71 (m, 2H), 2.39-2.47 (m, 5H), 1.99-2.12 (m, 2H), 1.77-1.80 (m, 2H); LCMS (m/z): 449.1 [M+H]⁺.

### Example 77: 1-[3-[[2-(1,2,3,4-Tetrahydroisoquinolin-7-ylamino)-5-(trifluoromethyl)pyrimidin-4-yl]amino]propyl]pyrrolidin-2-one

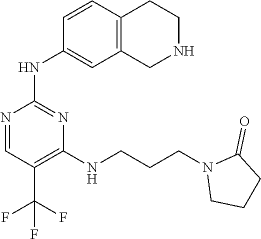 [SMILES: O=C1CCCN1CCCNc1nc(Nc2ccc3c(c2)CNCC3)ncc1C(F)(F)F]

Prepared analogously to Example 19 from 1-(3-((2-chloro-5-(trifluoromethyl)pyrimidin-4-yl)amino)propyl)pyrrolidin-2-one and tert-butyl 7-amino-3,4-dihydro-1H-isoquinoline-2-carboxylate. ¹H NMR (400 MHz, CHLOROFORM-d) d 8.12 (s, 1H), 6.91-7.00 (m, 1H), 6.50-6.57 (m, 2H), 6.03-6.13 (m, 1H), 4.82 (s, 2H), 4.01 (t, J=5.95 Hz, 2H), 3.49-3.55 (m, 2H), 3.33-3.44 (m, 4H), 2.76-2.84 (m, 2H), 2.38-2.47 (m, 2H), 2.01-2.11 (m, 2H), 1.77-1.86 (m, 2H). LCMS (m/z): 435.1 [M+H]⁺.

### Example 78: N2-(6-methoxy-1,2,3,4-tetrahydroisoquinolin-7-yl)-N4-(morpholin-2-ylmethyl)-5-(trifluoromethyl)pyrimidine-2,4-diamine

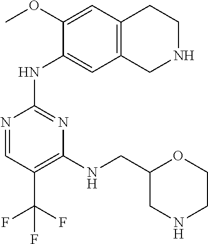 [SMILES: COc1cc2c(cc1Nc1ncc(C(F)(F)F)c(NCC3CNCCO3)n1)CNCC2]

### Step 1: Tert-butyl 7-[[4-chloro-5-(trifluoromethyl)pyrimidin-2-yl]amino]-6-methoxy-3,4-dihydro-1H-isoquinoline-2-carboxylate

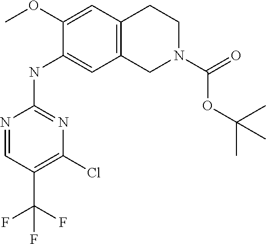 [SMILES: COc1cc2c(cc1Nc1ncc(C(F)(F)F)c(Cl)n1)CN(C(=O)OC(C)(C)C)CC2]

To a solution of 2,4-dichloro-5-(trifluoromethyl)pyrimidine (0.185 g, 0.853 mmol) in DCE (8 mL) and ^{t}BuOH (8 mL) was added ZnCl₂ dropwise at 5° C. and the reaction mixture left to stir for 1 hour. To this was added a solution of tert-butyl 7-amino-6-methoxy-3,4-dihydro-1H-isoquinoline-2-carboxylate (0.237 g, 0.853 mmol) and Et₃N (0.14 mL, 0.938 mmol) and the reaction mixture left to stir overnight at room temperature. The reaction mixture was concentrated in vacuo onto silica and purified by column chromatography (0-10% EtOAc in PE), affording the product (0.160 g, 41%). ¹H NMR (400 MHz, CHLOROFORM-d) d 8.57-8.63 (m, 1H), 8.10-8.14 (m, 1H), 6.66-6.69 (m, 1H), 4.54-4.59 (m, 2H), 3.89-3.90 (m, 3H), 3.63-3.69 (m, 2H), 2.78-2.85 (m, 2H), 1.50-1.52 (m, 9H); MS (m/z): 458.9 [M+H]⁺.

### Step 2: N2-(6-methoxy-1,2,3,4-tetrahydroisoquinolin-7-yl)-N4-(morpholin-2-ylmethyl)-5-(trifluoromethyl)pyrimidine-2,4-diamine

Tert-butyl-7-[[4-chloro-5-(trifluoromethyl)pyrimidin-2-yl]amino]-6-methoxy-3,4-dihydro-1H-isoquinoline-2-carboxylate (0.050 g, 0.109 mmol), tert-butyl 2-(aminomethyl)morpholine-4-carboxylate (0.023 g, 0.109 mmol), Et₃N (24 mL, 0.164 mmol) and IPA (2 mL) were combined and sealed in a microwave vial and heated at 80° C. for 18 hours. The compound was purified via column chromatography (10-40% EtOAc in PE). The fractions containing product were combined and concentrated in vacuo. This residue was taken up in DCM (1 mL) and TFA (1 mL) added. The reaction mixture was passed through an SCX cartridge and the product eluted with 2M NH₃ in MeOH. The eluent was concentrated in vacuo and triturated using Et₂O to give the product (0.030 g, 58% Yield). ¹H NMR (400 MHz, DMSO-d6) d 8.14 (s, 1H), 7.99 (s, 1H), 7.75 (br. s, 1H), 6.98-7.10 (m, 1H), 6.73 (s, 1H), 3.78-3.86 (m, 5H), 3.69-3.75 (m, 1H), 3.55-3.64 (m, 1H), 3.45-3.50 (m, 1H), 3.36-3.40 (m, 2H), 2.92-2.99 (m, 2H), 2.60-2.73 (m, 5H), 2.31-2.39 (m, 1H); LCMS (m/z): 439.0 [M+H]⁺.

Example 79: 3-[[2-[(6-Methoxy-1,2,3,4-tetrahydroisoquinolin-7-yl)amino]-5-(trifluoromethyl)pyrimidin-4-yl]amino]-1-morpholino-propan-1-one

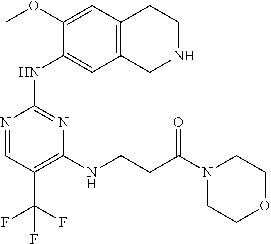 [SMILES: COc1cc2c(cc1Nc1ncc(C(F)(F)F)c(NCCC(=O)N3CCOCC3)n1)CNCC2]

### Step 1: 3-[[2-chloro-5-(trifluoromethyl)pyrimidin-4-yl]amino]-1-morpholino-propan-1-one

To 3-amino-1-morpholino-propan-1-one (0.732 g, 4.63 mmol) and Et₃N (1.34 mL, 9.26 mmol) in IPA was added 2,4-dichloro-5-(trifluoromethyl)pyrimidine (1.0 g, 4.67 mmol) and the reaction mixture heated at 50° C. overnight. The reaction mixture was cooled, concentrated in vacuo onto silica and purified via column chromatography (0-100% EtOAc in PE). The fractions containing product were combined and concentrated in vacuo to give the product, 3-[[2-chloro-5-(trifluoromethyl)pyrimidin-4-yl]amino]-1-morpholino-propan-1-one (0.419 g, 1.24 mmol, 27% Yield), as a white solid. ¹H NMR (400 MHz, CHLOROFORM-d) d 8.22 (s, 1H), 6.68-6.79 (m, 1H), 3.86-3.92 (m, 2H), 3.65-3.69 (m, 4H), 3.60-3.64 (m, 2H), 3.42-3.46 (m, 2H), 2.59-2.63 (m, 2H); LCMS (m/z): 339.0 [M+H]⁺.

### Step 2: 3-[[2-[(6-methoxy-1,2,3,4-tetrahydroisoquinolin-7-yl)amino]-5-(trifluoromethyl)pyrimidin-4-yl]amino]-1-morpholino-propan-1-one

3-[[2-chloro-5-(trifluoromethyl)pyrimidin-4-yl]amino]-1-morpholino-propan-1-one (0.050 g, 0.148 mmol), tert-butyl 7-amino-6-methoxy-3,4-dihydro-1H-isoquinoline-2-carboxylate (0.045 g, 0.163 mmol) and IPA (2 mL) were combined and heated at 80° C. for 3 hours. The reaction mixture was cooled, concentrated in vacuo and taken up in DCM, after which TFA (1 mL) was added and the reaction mixture left to stir for 30 minutes. The reaction mixture was concentrated in vacuo, taken up in MeOH and concentrated to give the product (0.011 g, 0.023 mmol, 15% Yield). ¹H NMR (400 MHz, DMSO-d6) d 8.15 (s, 1H), 7.97 (s, 1H), 7.68 (s, 1H), 7.01-7.13 (m, 1H), 6.70 (s, 1H), 3.79 (s, 3H), 3.71 (s, 2H), 3.58-3.67 (m, 2H), 3.48-3.54 (m, 4H), 3.42-3.46 (m, 2H), 3.35-3.39 (m, 2H), 2.86-2.93 (m, 2H), 2.58-2.66 (m, 4H); LCMS (m/z): 481.0 [M+H]⁺.

### Example 80: 1-Morpholino-3-[[2-[3-(4-piperidyloxy)anilino]-5-(trifluoromethyl)pyrimidin-4-yl]amino]propan-1-one

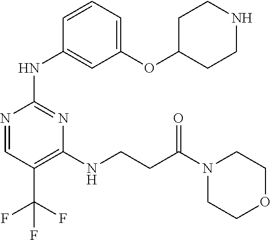 [SMILES: O=C(CCNc1nc(Nc2cccc(OC3CCNCC3)c2)ncc1C(F)(F)F)N1CCOCC1]

Prepared analogously ¹H NMR (400 MHz, DMSO-d6) d 9.51-9.59 (m, 1H), 8.20 (s, 1H), 7.35-7.40 (m, 1H), 7.26-7.33 (m, 1H), 7.03-7.15 (m, 2H), 6.51-6.61 (m, 1H), 4.24-4.34 (m, 1H), 3.64-3.73 (m, 2H), 3.51-3.54 (m, 2H), 3.43-3.50 (m, 4H), 3.35-3.38 (m, 2H), 2.90-2.97 (m, 2H), 2.63-2.70 (m, 2H), 2.51-2.57 (m, 2H), 1.85-1.96 (m, 2H), 1.36-1.49 (m, 2H); LCMS (m/z): 495 [M+H]⁺.

### Example 81: 1-[3-[[2-[(6-Methoxy-1,2,3,4-tetrahydroisoquinolin-7-yl)amino]-5-(trifluoromethyl)pyrimidin-4-yl]amino]propyl]pyrrolidin-2-one

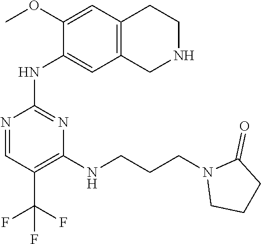 [SMILES: COc1cc2c(cc1Nc1ncc(C(F)(F)F)c(NCCCN3CCCC3=O)n1)CNCC2]

Tert-butyl-7-[[4-chloro-5-(trifluoromethyl)pyrimidin-2-yl]amino]-6-methoxy-3,4-dihydro-1H-isoquinoline-2-carboxylate (0.040 g, 0.087 mmol), 1-(3-aminopropyl)pyrrolidin-2-one (0.015 g, 0.105 mmol) and IPA (2 mL) were combined and heated at 80° C. for three hours. The reaction mixture was cooled, concentrated in vacuo and taken up in DCM, after which TFA (1 mL) was added and the reaction mixture left to stir for 30 minutes. The reaction mixture was concentrated in vacuo, taken up in MeOH and concentrated to give the product (3 mg, 0.006 mmol, 7.4% Yield). ¹H NMR (400 MHz, DMSO-d6) d 8.15 (s, 1H), 7.97 (s, 1H), 7.74 (s, 1H), 7.10-7.23 (m, 1H), 6.73 (s, 1H), 3.77-3.84 (m, 5H), 3.29-3.35 (m, 4H), 3.17-3.23 (m, 2H), 2.91-3.01 (m, 2H), 2.62-2.71 (m, 2H), 2.18-2.26 (m, 2H), 1.85-1.97 (m, 2H), 1.65-1.76 (m, 2H); LCMS (m/z): 465.0 [M+H]⁺.

### Example 82: N-[3-[[2-[(6-methoxy-1,2,3,4-tetrahydroisoquinolin-7-yl)amino]-5-(trifluoromethyl)pyrimidin-4-yl]amino]propyl]cyclobutanecarboxamide

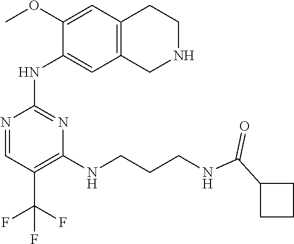 [SMILES: COc1cc2c(cc1Nc1ncc(C(F)(F)F)c(NCCCNC(=O)C3CCC3)n1)CNCC2]

Prepared analogously to Example 81 from Tert-butyl-7-[[4-chloro-5-(trifluoromethyl)pyrimidin-2-yl]amino]-6-methoxy-3,4-dihydro-1H-isoquinoline-2-carboxylate and N-(3-aminopropyl)cyclobutene-carboxamide. ¹H NMR (400 MHz, DMSO-d6) d 8.10 (s, 1H), 7.90 (s, 1H), 7.75 (br. s, 1H), 7.60-7.66 (m, 1H), 7.13-7.22 (m, 1H), 6.69 (s, 1H), 3.72-3.78 (m, 5H), 3.33-3.39 (m, 2H), 2.99-3.06 (m, 2H), 2.87-2.94 (m, 3H), 2.58-2.65 (m, 2H), 2.03-2.12 (m, 2H), 1.91-1.99 (m, 2H), 1.77-1.89 (m, 1H), 1.66-1.74 (m, 1H), 1.56-1.64 (m, 2H); LCMS (m/z): 479.0 [M+H]⁺.

### Example 83: 1-[3-[[2-(1,2,3,4-Tetrahydroisoquinolin-7-ylamino)-5-(trifluoromethyl)pyrimidin-4-yl]amino]propyl]piperidin-2-one

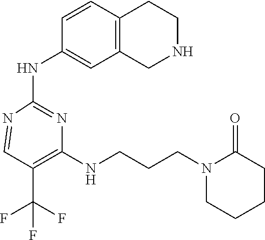 [SMILES: O=C1CCCCN1CCCNc1nc(Nc2ccc3c(c2)CNCC3)ncc1C(F)(F)F]

Prepared analogously to Example 19 from Intermediate 1 and tert-butyl 7-amino-3,4-dihydro-1H-isoquinoline-2-carboxylate. ¹H NMR (400 MHz, CHLOROFORM-d) d 8.12 (s, 1H), 6.95 (d, J=8.70 Hz, 1H), 6.51-6.58 (m, 2H), 6.21-6.33 (m, 1H), 4.83 (s, 2H), 3.95-4.08 (m, 2H), 3.43-3.53 (m, 4H), 3.25-3.32 (m, 2H), 2.80 (s, 2H), 2.44 (t, J=5.95 Hz, 2H), 1.78-1.86 (m, 6H); LCMS (m/z): 449.1 [M+H]⁺.

### Example 84: 5-Cyclopropyl-N4-(3-(dimethylamino)propyl)-N2-(3-(1-methylpiperidin-4-yloxy)phenyl)pyrimidine-2,4-diamine

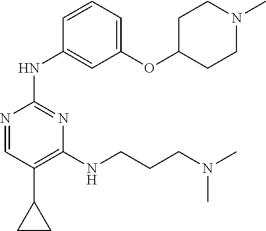 [SMILES: CN(C)CCCNc1nc(Nc2cccc(OC3CCN(C)CC3)c2)ncc1C1CC1]

### Step 1: N1-(2-Chloro-5-cyclopropylpyrimidin-4-yl)-N3, N3-dimethylpropane-1, 3-diamine

To a solution of 2,4-dichloro-5-cyclopropylpyrimidine (0.25 g, 1.32 mmol) in IPA (5 mL) was added DIPEA (0.34 mL, 1.99 mmol) followed by N1,N1-dimethylpropane-1,3-diamine (0.16 g, 1.59 mmol) at room temperature and stirred for 16 h at 60° C. The reaction mixture was evaporated, the obtained residue was taken in 10% methanol/DCM (30 mL) and washed with water (10 mL), and the separated organic layer was dried over sodium sulfate and concentrated in vacuo. The crude compound was purified by column chromatography (silica gel, 100-200 mesh, eluted with 1% methanol/chloroform) to obtain an off-white solid (0.15 g, 45%). ¹H NMR (400 MHz, CDCl₃): δ 7.8 (s, 1H), 7.71 (s, 1H), 3.62-3.58 (m, 2H), 2.52-2.49 (m, 2H), 2.27 (s, 6H), 1.81-1.75 (m, 2H), 1.37-1.34 (m, 1H), 0.89-0.85 (m, 2H), 0.56-0.52 (m, 2H); LCMS (m/z): 255 [M+H]⁺.

### Step 2: 5-Cyclopropyl-N4-(3-(dimethylamino)propyl)-N2-(3-(1-methylpiperidin-4-yloxy)phenyl)-pyrimidine-2,4-diamine

To a solution of N1-(2-chloro-5-cyclopropylpyrimidin-4-yl)-N3,N3-dimethylpropane-1,3-diamine (0.15 g, 0.59 mmol) in t-butanol (4 mL) was added TFA (0.27 g, 2.36 mmol) followed by 3-[(1-methyl-4-piperidyl)oxy]aniline (0.097 g, 0.47 mmol) at room temperature and stirred at 100° C. for 4 h. The reaction mixture was evaporated, the obtained residue was basified with 1N NaOH solution, extracted with ethyl acetate (2×20 mL) combined organic layer was washed with brine solution, dried over sodium sulfate and concentrated in vacuo. The crude solid was purified by triturating with diethyl ether and n-pentane to obtain the product as an off white solid (0.06 g, 24%). ¹H NMR (400 MHz, CDCl₃): δ 7.68 (s, 1H), 7.37.38 (m, 1H), 7.15-7.12 (m, 2H), 6.88-6.81 (m, 2H), 6.56-6.53 (m, 1H), 4.40-4.35 (m, 1H), 3.63-3.59 (m, 2H), 2.95 (d, J=10.8 Hz, 2H), 2.60 (d, J=10.8 Hz, 1H), 2.49-2.46 (m, 2H), 2.29-2.16 (m, 8H), 2.1-1.8 (m, 3H), 1.79-1.64 (m, 3H), 1.63-1.35 (m, 3H), 0.83-0.78 (m, 2H), 0.51-0.47 (m, 2H); LCMS (m/z): 425 [M+H]⁺.

### Example 85: N4-(1H-pyrazol-3-ylmethyl)-N2-(1-pyrrolidin-3-ylpyrazol-4-yl)-5-(trifluoromethyl)pyrimidine-2,4-diamine

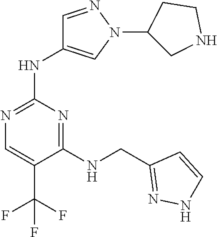 [SMILES: FC(F)(F)c1cnc(Nc2cnn(C3CCNC3)c2)nc1NCc1cc[nH]n1]

2-Chloro-5-(trifluoromethyl)-N-[(1-tritylpyrazol-3-yl)methyl]pyrimidin-4-amine (50 mg, 0.09 mmol) and tert-butyl 3-(4-aminopyrazol-1-yl)pyrrolidine-1-carboxylate (27 mg, 0.11 mmol) were combined in butanol in a microwave vial and heated at 100° C. overnight. The reaction mixture was cooled and TFA added. The reaction mixture was passed through SCX cartridge with the product being eluted with 2M NH₃ in MeOH. The eluent was concentrated in vacuo, taken up in MeOH and purified by preparative HPLC. ¹H NMR (400 MHz, METHANOL-D3) 8.09 (br. s., 1H), 7.82 (br. s., 1H), 7.55 (br. s., 1H), 7.47 (br. s., 1H), 6.21 (br. s., 1H), 4.63-4.88 (m, 3H), 3.23 (br. s., 1H), 2.95-3.13 (m, 1H), 2.30 (d, J=7.79 Hz, 1H), 1.97-2.22 (m, 1H). LCMS (m/z) 394 [M+H]⁺.

### Example 86: N4-(1H-pyrazol-3-ylmethyl)-N2-[4-(2-pyrrolidin-1-ylethyl)phenyl]-5-(trifluoromethyl)pyrimidine-2,4-diamine

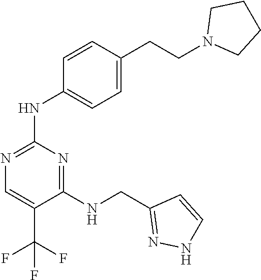 [SMILES: FC(F)(F)c1cnc(Nc2ccc(CCN3CCCC3)cc2)nc1NCc1cc[nH]n1]

2-Chloro-5-(trifluoromethyl)-N-[(1-tritylpyrazol-3-yl)methyl]pyrimidin-4-amine (50 mg, 0.09 mmol) and 4-(ethylenepyrrolidine) aniline (19 mg, 0.10 mmol) were combined in butanol (3 mL) in a sealed vial and heated at 110° C. for 18 h. The reaction mixture was cooled, concentrated in vacuo, TFA added, stirred for 30 min. Et₃SiH was added and the reaction mixture concentrated in vacuo. The residue was taken up in MeOH and passed through SCX cartridge with the product eluted with 2M NH₃ in MeOH. The product was purified by preparative HPLC. ¹H NMR (DMSO-d₆) d: 9.51 (br. s., 1H), 8.18 (s, 1H), 7.32-7.66 (m, 4H), 7.06 (d, J=8.2 Hz, 2H), 6.04-6.16 (m, 1H), 4.62 (d, J=5.5 Hz, 2H), 2.62-2.68 (m, 2H), 2.53-2.61 (m, 2H), 2.43-2.47 (m, 4H), 1.56-1.73 (m, 5H); LCMS (m/z): 432 [M+H]⁺.

### Example 87: N2-(4-morpholinophenyl)-N4-(pyrrolidin-3-ylmethyl)-5-(trifluoromethyl)pyrimidine-2,4-diamine

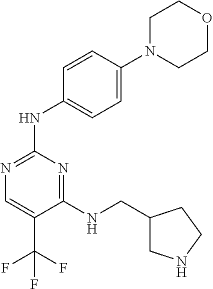 [SMILES: FC(F)(F)c1cnc(Nc2ccc(N3CCOCC3)cc2)nc1NCC1CCNC1]

### Step 1: 4-chloro-N-(4-morpholinophenyl)-5-(trifluoromethyl)pyrimidin-2-amine

Zinc chloride, 1M in diethyl ether (3 mL, 3 mmol) was added dropwise to a solution of pyridine in 1:1 DCE:^{t}BuOH (12 mL) with ice cooling. Stirred for 1 h. 4-Morpholinoaniline (246 mg, 1.4 mmol) was added. A solution of triethylamine in 1:1 DCE:^{t}BuOH (4 mL) was added dropwise. The solution was allowed to warm to room temperature with cooling still present. After 3 h, the reaction was complete. The reaction mixture was concentrated to dryness, the residue was preloaded onto silica and purified in 2:1 PE/EA to give 4-chloro-N-(4-morpholinophenyl)-5-(trifluoromethyl)pyrimidin-2-amine as a yellow solid (270 mg, 55%). ¹H NMR (400 MHz, DMSO-d6) 10.44 (s, 1H), 8.70 (s, 1H), 7.40-7.60 (m, 2H), 6.87-7.05 (m, 2H), 3.61-3.84 (m, 4H), 2.96-3.11 (m, 4H). LCMS (m/z): 358 [M+H]⁺.

### Step 2: N2-(4-morpholinophenyl)-N4-(pyrrolidin-3-ylmethyl)-5-(trifluoromethyl)pyrimidine-2,4-diamine

4-Chloro-N-(4-morpholinophenyl)-5-(trifluoromethyl)pyrimidin-2-amine (50 mg, 0.14 mmol), 3-aminopyrrolidine (28 mg, 0.14 mmol) and triethylamine (30 μl, 0.21 mmol) were combined in butanol, sealed in a chromacol vial and heated at 105° C. overnight. The reaction mixture was concentrated in vacuo, passed through SCX and submitted for HPLC purification (pH10) to give the product N2-(4-morpholinophenyl)-N4-(pyrrolidin-3-ylmethyl)-5-(trifluoromethyl)pyrimidine-2,4-diamine (10.8 mg, 18%). ¹H NMR (400 MHz, CHLOROFORM-d) d 8.13 (d, J=0.92 Hz, 1H), 7.43-7.50 (m, 2H), 7.16 (s, 1H), 6.85-6.94 (m, 2H), 5.77 (br. s., 1H), 3.87 (dd, J=3.89, 5.72 Hz, 4H), 3.41-3.56 (m, 2H), 3.10-3.16 (m, 4H), 2.99-3.08 (m, 2H), 2.87-2.96 (m, 1H), 2.78 (dd, J=4.81, 10.76 Hz, 1H), 2.44-2.54 (m, 1H), 1.97 (dtd, J=5.04, 8.30, 13.17 Hz, 1H), 1.46-1.57 (m, 1H). LCMS (m/z): 423 [M+H]⁺.

Examples 88-92 were prepared analogously to Example 87:

### Example 88: N4-(4-aminocyclohexyl)-N2-(4-morpholinophenyl)-5-(trifluoromethyl)pyrimidine-2,4-diamine

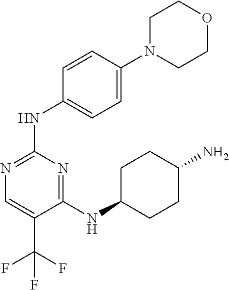 [SMILES: N[C@H]1CC[C@H](Nc2nc(Nc3ccc(N4CCOCC4)cc3)ncc2C(F)(F)F)CC1]

¹H NMR (400 MHz, CHLOROFORM-d) d 8.12 (s, 1H), 7.43-7.53 (m, 2H), 7.05 (br. s., 1H), 6.87-6.94 (m, 2H), 4.91 (d, J=6.41 Hz, 1H), 3.95-4.05 (m, 1H), 3.95-4.05 (m, J=3.90, 6.60 Hz, 1H), 3.84-3.92 (m, 4H), 3.09-3.18 (m, 4H), 2.75-2.85 (m, 1H), 2.18 (d, J=8.70 Hz, 2H), 1.93-2.02 (m, 2H), 1.22-1.40 (m, 4H). LCMS (m/z): 437 [M+H]⁺.

### Example 89: N2-(4-morpholinophenyl)-N4-[2-(4-piperidyl)ethyl]-5-(trifluoromethyl)pyrimidine-2,4-diamine

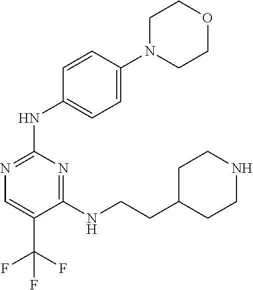 [SMILES: FC(F)(F)c1cnc(Nc2ccc(N3CCOCC3)cc2)nc1NCCC1CCNCC1]

¹H NMR (400 MHz, CHLOROFORM-d) d 8.12 (s, 1H), 7.43-7.53 (m, 2H), 7.05 (br. s., 1H), 6.87-6.94 (m, 2H), 4.91 (d, J=6.41 Hz, 1H), 3.95-4.05 (m, 1H), 3.95-4.05, (m, J=3.90, 6.60 Hz, 1H), 3.84-3.92 (m, 4H), 3.09-3.18 (m, 4H), 2.75-2.85 (m, 1H), 2.18 (d, J=8.70 Hz, 2H), 1.93-2.02 (m, 2H), 1.22-1.40 (m, 4H). LCMS (m/z): 451 [M+H]⁺.

### Example 90: N2-(4-morpholinophenyl)-N4-(morpholin-2-ylmethyl)-5-(trifluoromethyl)pyrimidine-2,4-diamine

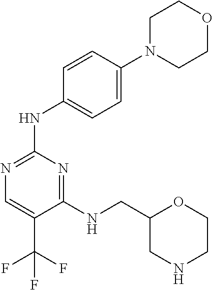 [SMILES: FC(F)(F)c1cnc(Nc2ccc(N3CCOCC3)cc2)nc1NCC1CNCCO1]

¹H NMR (400 MHz, CHLOROFORM-d) d 8.14 (d, J=0.92 Hz, 1H), 7.40-7.48 (m, 2H), 6.99-7.06 (m, 1H), 6.87-6.94 (m, 2H), 5.56 (br. s., 1H), 3.90-3.96 (m, 1H), 3.83-3.90 (m, 4H), 3.59-3.75 (m, 3H), 3.37-3.48 (m, 1H), 3.07-3.18 (m, 4H), 2.82-2.97 (m, 3H), 2.65 (dd, J=9.85, 12.14 Hz, 1H). LCMS (m/z): 439 [M+H]⁺.

### Example 91: N4-[2-(1H-imidazol-4-yl)ethyl]-N2-(4-morpholinophenyl)-5-(trifluoromethyl)pyrimidine-2,4-diamine

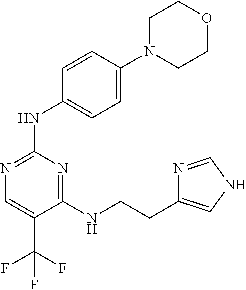 [SMILES: FC(F)(F)c1cnc(Nc2ccc(N3CCOCC3)cc2)nc1NCCc1c[nH]cn1]

¹H NMR (400 MHz, CHLOROFORM-d) d 8.11 (s, 1H), 7.66 (s, 1H), 7.45-7.52 (m, 2H), 6.87-6.94 (m, 2H), 6.84 (d, J=0.92 Hz, 1H), 5.91 (br. s., 1H), 3.86-3.89 (m, 4H), 3.88 (d, J=4.58 Hz, 4H), 3.75-3.83 (m, 2H), 3.11-3.14 (m, 4H), 2.94 (t, J=6.41 Hz, 2H). LCMS (m/z): 434 [M+H]⁺.

### Example 92: N2-(4-morpholinophenyl)-N4-(1H-pyrazol-4-ylmethyl)-5-(trifluoromethyl)pyrimidine-2,4-diamine

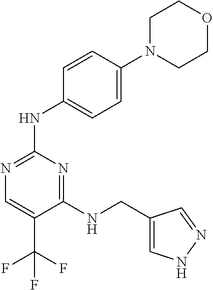 [SMILES: FC(F)(F)c1cnc(Nc2ccc(N3CCOCC3)cc2)nc1NCc1cn[nH]c1]

¹H NMR (400 MHz, CHLOROFORM-d) d 8.12-8.20 (m, 1H), 7.55-7.60 (m, 2H), 7.45-7.50 (m, 2H), 6.88-6.94 (m, 2H), 4.60-4.64 (m, 2H), 3.85-3.91 (m, 4H), 3.10-3.16 (m, 4H). LCMS (m/z): 420 [M+H]⁺.

### Example A

ULK activity was measured using a radiometric assay, to measure the incorporation of radiolabelled ³³P onto MBP substrate using a glass fiber capture filter method. Reaction conditions were 0.2 mg/mL MBP, 20 uM ATP (0.25 μCi/well), 50 mM Tris HCl, pH7.5, 10 mM MgCl₂, 0.1% beta-mercaptoethanol, 0.1 mM EGTA, 0.01% BSA. Ten point Half log Compound dilution series were prepared in 100% DMSO and added to the reaction to give final assay concentration of 10% DMSO. Compounds were tested in duplicate and values normalized to 10% DMSO only controls. The data were fitted to a four parameter fit equation and the IC50 values shown are averages of at least two independent experiments.

TABLE 1
ULK Biochemical activity

| Example | IC₅₀ |
|---|---|
| 1 | A |
| 2 | A |
| 3 | A |
| 4 | A |
| 5 | C |
| 6 | B |
| 7 | A |
| 8 | A |
| 9 | A |
| 10 | A |
| 11 | A |
| 12 | A |
| 13 | A |
| 14 | A |
| 15 | C |
| 16 | A |
| 17 | A |
| 18 | A |
| 19 | A |
| 20 | A |
| 21 | A |
| 22 | C |
| 23 | A |
| 24 | A |
| 25 | A |
| 26 | A |
| 27 | A |
| 28 | A |
| 29 | B |
| 30 | C |
| 31 | C |
| 32 | C |
| 33 | C |
| 34 | C |
| 35 | C |
| 36 | C |
| 37 | C |
| 38 | C |
| 39 | C |
| 40 | C |
| 41 | B |
| 42 | B |
| 43 | A |
| 44 | B |
| 45 | A |
| 46 | B |
| 47 | C |
| 48 | B |
| 49 | C |
| 50 | C |
| 51 | C |
| 52 | B |
| 53 | C |
| 54 | A |
| 55 | B |
| 56 | B |
| 57 | B |
| 58 | B |
| 59 | B |
| 60 | A |
| 61 | C |
| 62 | A |
| 63 | A |
| 64 | B |
| 65 | A |
| 66 | A |
| 67 | A |
| 68 | C |
| 69 | C |
| 70 | B |
| 71 | C |
| 72 | C |
| 73 | C |
| 74 | C |
| 75 | B |
| 76 | A |
| 77 | C |
| 78 | B |
| 79 | B |
| 80 | B |
| 81 | A |
| 82 | A |
| 83 | A |
| 84 | C |
| 85 | A |
| 86 | B |
| 87 | B |
| 88 | B |
| 89 | B |
| 90 | B |
| 91 | B |
| 92 | C |

A = IC₅₀ less than 200 nM
B = IC₅₀ is more than or equal to 200 nM and less than 1 μM
C = IC₅₀ is more than or equal to 1 μM

1. A compound of Formula (I), or a pharmaceutically acceptable salt, solvate, or stereoisomer thereof: 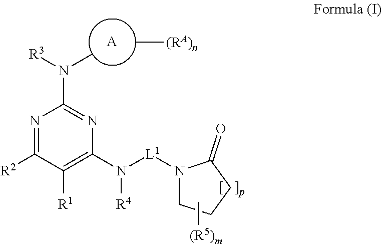
wherein:
R¹ is C₁-C₆haloalkyl or cycloalkyl;
R² is hydrogen, halogen, deuterium, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, or heterocycloalkyl;
R³ is hydrogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl;
R⁴ is hydrogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl;
each R⁵ is independently deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R⁵ on the same carbon are taken together to form an oxo;
Ring A is a 6- to 12-membered bicyclic ring optionally comprising 1-4 heteroatoms selected from the group consisting of O, S, N, P, and B;
each R^{A} is independently deuterium, halogen, —CN, —NO₂, —OH, —OR^{a}, —O(C₂-C₆alkylene)OR^{a}, —O(C₂-C₆alkylene)NR^{c}R^{d}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —NR^{b}C(═O)OR^{b}, —NR^{b}C(═O)NR^{c}R^{d}, —NHS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl are independently substituted with one, two, or three R^{Aa};
or two R^{A} on the same carbon are taken together to form an oxo;
each R^{Aa} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —NR^{b}C(═O)OR^{b}, —NR^{b}C(═O)NR^{c}R^{d}, —NHS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{Aa} on the same carbon are taken together to form an oxo;
L¹ is a C₃-C₄ alkylene optionally substituted with one, two, or three R^{L1};
each R^{L1} is independently deuterium, halogen, —CN, —NO₂, —OH, —OR^{a}, or —NR^{c}R^{d};
or two R^{L1} on the same carbon are taken together to form an oxo;
n is 1-4;
m is 0-4;
p is 1 or 2;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl;
or R^{c} and R^{d} are taken together with the atom to which they are attached to form a heterocycloalkyl optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl.

2. The compound of claim 1, or a pharmaceutically acceptable salt, solvate, or stereoisomer thereof; wherein:
R² is hydrogen.

3. The compound of claim 1 or 2, or a pharmaceutically acceptable salt, solvate, or stereoisomer thereof; wherein:
R³ is hydrogen or C₁-C₆alkyl.

4. The compound of claim 1 or 2, or a pharmaceutically acceptable salt, solvate, or stereoisomer thereof; wherein:
R³ is hydrogen.

5. The compound of any one of claims 1-4, or a pharmaceutically acceptable salt, solvate, or stereoisomer thereof; wherein:
R⁴ is hydrogen or C₁-C₆alkyl.

6. The compound of any one of claims 1-4, or a pharmaceutically acceptable salt, solvate, or stereoisomer thereof; wherein:
R⁴ is hydrogen.

7. The compound of any one of claims 1-6, or a pharmaceutically acceptable salt, solvate, or stereoisomer thereof; wherein:
R¹ is C₁-C₆haloalkyl.

8. The compound of claim 7, or a pharmaceutically acceptable salt, solvate, or stereoisomer thereof; wherein:
R¹ is CF₃.

9. The compound of any one of claims 1-6, or a pharmaceutically acceptable salt, solvate, or stereoisomer thereof; wherein:
R¹ is cycloalkyl.

10. The compound of claim 9, or a pharmaceutically acceptable salt, solvate, or stereoisomer thereof; wherein:
R¹ is cyclopropyl.

11. The compound of any one of claims 1-10, or a pharmaceutically acceptable salt, solvate, or stereoisomer thereof; wherein:
L¹ is C₃-C₄ alkylene.

12. The compound of any one of claims 1-11, or a pharmaceutically acceptable salt, solvate, or stereoisomer thereof; wherein:
L¹ is C₃ alkylene.

13. The compound of any one of claims 1-12, or a pharmaceutically acceptable salt, solvate, or stereoisomer thereof; wherein:
p is 1.

14. The compound of any one of claims 1-12, or a pharmaceutically acceptable salt, solvate, or stereoisomer thereof; wherein:
p is 2.

15. The compound of any one of claims 1-14, or a pharmaceutically acceptable salt, solvate, or stereoisomer thereof; wherein:
m is 0.

16. The compound of any one of claims 1-15, or a pharmaceutically acceptable salt, solvate, or stereoisomer thereof; wherein:
Ring A is a 6- to 12-membered bicyclic ring optionally comprising 1-4 heteroatoms selected from the group consisting of O, S, and N.

17. The compound of any one of claims 1-15, or a pharmaceutically acceptable salt, solvate, or stereoisomer thereof; wherein:
Ring A is a 6- to 12-membered bicyclic ring optionally comprising 1-4 heteroatoms selected from the group consisting of O and N.

18. The compound of any one of claims 1-15, or a pharmaceutically acceptable salt, solvate, or stereoisomer thereof; wherein:
Ring A is a 6- to 12-membered bicyclic ring optionally comprising 1-4 heteroatoms selected from the group consisting of O and N.

19. The compound of any one of claims 1-15, or a pharmaceutically acceptable salt, solvate, or stereoisomer thereof; wherein:
Ring A is a 6- to 12-membered bicyclic ring comprising 1 or 2 heteroatoms selected from the group consisting of O and N.

20. The compound of any one of claims 1-15, or a pharmaceutically acceptable salt, solvate, or stereoisomer thereof; wherein:
Ring A is a 6- to 10-membered bicyclic ring comprising 1 heteroatom that is O.

21. The compound of any one of claims 1-15, or a pharmaceutically acceptable salt, solvate, or stereoisomer thereof; wherein:
Ring A is a 6- to 10-membered bicyclic ring comprising 1 heteroatom that is N.

22. The compound of any one of claims 1-15, or a pharmaceutically acceptable salt, solvate, or stereoisomer thereof; wherein:
Ring A is

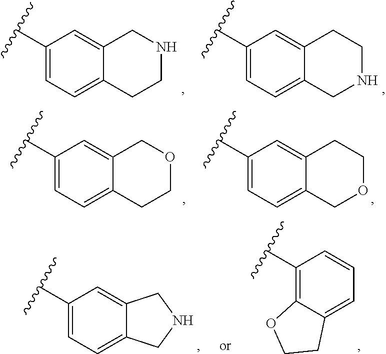

23. The compound of any one of claims 1-15, or a pharmaceutically acceptable salt, solvate, or stereoisomer thereof; wherein:
Ring A is

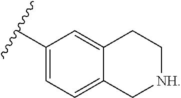 [SMILES: *c1ccc2c(c1)CCNC2]

24. The compound of any one of claims 1-15, or a pharmaceutically acceptable salt, solvate, or stereoisomer thereof; wherein: 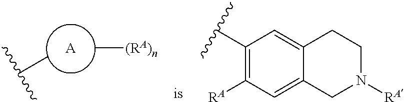
and R^{A′} is hydrogen or C₁-C₆alkyl.

25. The compound of any one of claims 1-24, or a pharmaceutically acceptable salt, solvate, or stereoisomer thereof; wherein:
each R^{A} is independently deuterium, halogen, —CN, —NO₂, —OH, —OR^{a}, —O(C₂-C₆alkylene)OR^{a}, —O(C₂-C₆alkylene)NR^{c}R^{d}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, cycloalkyl, heterocycloalkyl, aryl, and heteroaryl are independently substituted with one, two, or three R^{Aa}; or two R^{A} on the same carbon are taken together to form an oxo.

26. The compound of any one of claims 1-25, or a pharmaceutically acceptable salt, solvate, or stereoisomer thereof; wherein:
each R^{A} is independently halogen, —OH, —OR^{a}, —O(C₂-C₆alkylene)NR^{c}R^{d}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, cycloalkyl, or heterocycloalkyl; wherein each alkyl, cycloalkyl, and heterocycloalkyl are independently substituted with one, two, or three R^{Aa}; or two R^{A} on the same carbon are taken together to form an oxo.

27. The compound of any one of claims 1-26, or a pharmaceutically acceptable salt, solvate, or stereoisomer thereof; wherein:
each R^{A} is independently halogen, —OH, —OR^{a}, —NR^{c}R^{d}, —C(═O)R^{a}, C₁-C₆alkyl, or C₁-C₆haloalkyl; wherein each alkyl are independently substituted with one, two, or three R^{Aa}; or two R^{A} on the same carbon are taken together to form an oxo.

28. The compound of any one of claims 1-27, or a pharmaceutically acceptable salt, solvate, or stereoisomer thereof; wherein:
each R^{A} is independently halogen, —OH, —OR^{a}, —NR^{c}R^{d}, C₁-C₆alkyl, or C₁-C₆haloalkyl; wherein each alkyl are independently substituted with one, two, or three R^{Aa}.

29. The compound of any one of claims 1-28, or a pharmaceutically acceptable salt, solvate, or stereoisomer thereof; wherein:
each R^{A} is independently halogen or C₁-C₆alkyl.

30. The compound of any one of claims 1-29, or a pharmaceutically acceptable salt, solvate, or stereoisomer thereof; wherein:
each R^{A} is independently halogen.

31. The compound of any one of claims 1-30, or a pharmaceutically acceptable salt, solvate, or stereoisomer thereof; wherein:
each R^{Aa} is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl; or two R^{Aa} on the same carbon are taken together to form an oxo.

32. The compound of any one of claims 1-31, or a pharmaceutically acceptable salt, solvate, or stereoisomer thereof; wherein:
each R^{Aa} is independently deuterium, halogen, —CN, —OH, —OR^{a}, —NR^{c}R^{d}, C₁-C₆alkyl, C₁-C₆haloalkyl, or C₁-C₆deuteroalkyl; or two R^{Aa} on the same carbon are taken together to form an oxo.

33. The compound of any one of claims 1-32, or a pharmaceutically acceptable salt, solvate, or stereoisomer thereof; wherein:
each R^{Aa} is independently halogen, —OH, —OR^{a}, —NR^{c}R^{d}, or C₁-C₆alkyl.

34. The compound of any one of claims 1-33, or a pharmaceutically acceptable salt, solvate, or stereoisomer thereof; wherein:
n is 1-3.

35. The compound of any one of claims 1-34, or a pharmaceutically acceptable salt, solvate, or stereoisomer thereof; wherein:
n is 1 or 2.

36. The compound of any one of claims 1-34, or a pharmaceutically acceptable salt, solvate, or stereoisomer thereof; wherein:
n is 1.

37. The compound of any one of claims 1-34, or a pharmaceutically acceptable salt, solvate, or stereoisomer thereof; wherein:
n is 2.

38. The compound of any one of claims 1-34, or a pharmaceutically acceptable salt, solvate, or stereoisomer thereof; wherein:
n is 3.

39. A compound of Formula (IIa) or (IIb), or a pharmaceutically acceptable salt, solvate, or stereoisomer thereof: 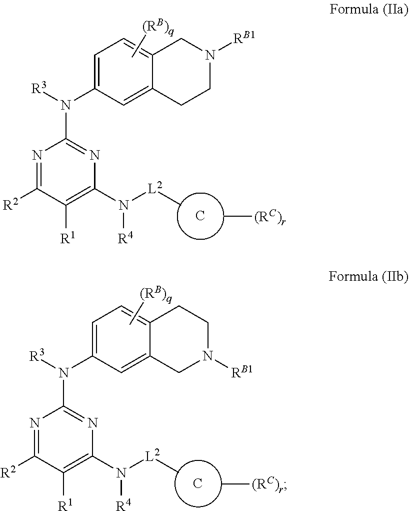
wherein:
R¹ is C₁-C₆haloalkyl or cycloalkyl;
R² is hydrogen, halogen, deuterium, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, or heterocycloalkyl;
R³ is hydrogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl;
R⁴ is hydrogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl;
each R^{B} is independently deuterium, halogen, —CN, —NO₂, —OH, —OR^{a}, —O(C₂-C₆alkylene)OR^{a}, —O(C₂-C₆alkylene)NR^{c}R^{d}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —NR^{b}C(═O)OR^{b}, —NR^{b}C(═O)NR^{c}R^{d}, —NHS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl are independently substituted with one, two, or three R^{Ba};
or two R^{B} on the same carbon are taken together to form an oxo;
each R^{Ba} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —NR^{b}C(═O)OR^{b}, —NR^{b}C(═O)NR^{c}R^{d}, —NHS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{Ba} on the same carbon are taken together to form an oxo;
R^{B1} is hydrogen, —S(═O)R^{a}, —S(═O)₂R^{a}, —S(═O)₂NR^{c}R^{d}, —C(═O)R^{a}, —C(═O)OR^{b}, —C(═O)NR^{C}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl);
Ring C is cycloalkyl, heterocycloalkyl, aryl, or heteroaryl;
each R^{C} is independently deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{C} on the same carbon are taken together to form an oxo;
L² is a C₁-C₄ alkylene optionally substituted with one, two, or three R^{L2};
each R^{L2} is independently deuterium, halogen, —CN, —NO₂, —OH, —OR^{a}, or —NR^{c}R^{d};
or two R^{L2} on the same carbon are taken together to form an oxo;
q is 0-4;
r is 0-4;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl;
or R^{c} and R^{d} are taken together with the atom to which they are attached to form a heterocycloalkyl optionally substituted with one, two, or three deuterium, oxo, halogen, —CN, —OH, —OCH₃, —S(═O)CH₃, —S(═O)₂CH₃, —NH₂, —NHCH₃, —N(CH₃)₂, —S(═O)₂NH₂, —C(═O)CH₃, —C(═O)OH, —C(═O)OCH₃, C₁-C₆alkyl, C₁-C₆deuteroalkyl, C₁-C₆haloalkyl, C₁-C₆hydroxyalkyl, or C₁-C₆aminoalkyl.

40. The compound of claim 39, or a pharmaceutically acceptable salt, solvate, or stereoisomer thereof; wherein the compound is of Formula (IIa): 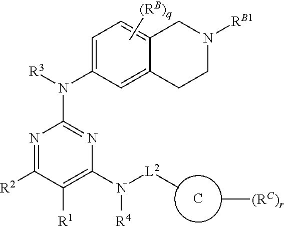
Formula (IIa).

41. The compound of claim 39, or a pharmaceutically acceptable salt, solvate, or stereoisomer thereof; wherein the compound is of Formula (IIb): 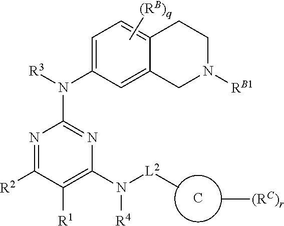
Formula (IIb).

42. The compound of any one of claims 39-41, or a pharmaceutically acceptable salt, solvate, or stereoisomer thereof; wherein:
R² is hydrogen.

43. The compound of any one of claims 39-42, or a pharmaceutically acceptable salt, solvate, or stereoisomer thereof; wherein:
R³ is hydrogen or C₁-C₆alkyl.

44. The compound of any one of claims 39-43, or a pharmaceutically acceptable salt, solvate, or stereoisomer thereof; wherein:
R³ is hydrogen.

45. The compound of any one of claims 39-44, or a pharmaceutically acceptable salt, solvate, or stereoisomer thereof; wherein:
R⁴ is hydrogen or C₁-C₆alkyl.

46. The compound of any one of claims 39-45, or a pharmaceutically acceptable salt, solvate, or stereoisomer thereof; wherein:
R⁴ is hydrogen.

47. The compound of any one of claims 39-46, or a pharmaceutically acceptable salt, solvate, or stereoisomer thereof; wherein:
R¹ is C₁-C₆haloalkyl.

48. The compound of claim 47, or a pharmaceutically acceptable salt, solvate, or stereoisomer thereof; wherein:
R¹ is CF₃.

49. The compound of any one of claims 39-46, or a pharmaceutically acceptable salt, solvate, or stereoisomer thereof; wherein:
R¹ is cycloalkyl.

50. The compound of claim 49, or a pharmaceutically acceptable salt, solvate, or stereoisomer thereof; wherein:
R¹ is cyclopropyl.

51. The compound of any one of claims 39-50, or a pharmaceutically acceptable salt, solvate, or stereoisomer thereof; wherein:
L² is C₃-C₄ alkylene.

52. The compound of any one of claims 39-51, or a pharmaceutically acceptable salt, solvate, or stereoisomer thereof; wherein:
L² is C₃ alkylene.

53. The compound of any one of claims 39-52, or a pharmaceutically acceptable salt, solvate, or stereoisomer thereof; wherein:
q is 0-2.

54. The compound of any one of claims 39-53, or a pharmaceutically acceptable salt, solvate, or stereoisomer thereof; wherein:
q is 0 or 1.

55. The compound of any one of claims 39-53, or a pharmaceutically acceptable salt, solvate, or stereoisomer thereof; wherein:
q is 1 or 2.

56. The compound of any one of claims 39-53, or a pharmaceutically acceptable salt, solvate, or stereoisomer thereof; wherein:
q is 1.

57. The compound of any one of claims 39-56, or a pharmaceutically acceptable salt, solvate, or stereoisomer thereof; wherein:

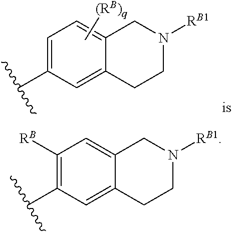

58. The compound of any one of claims 39-56, or a pharmaceutically acceptable salt, solvate, or stereoisomer thereof; wherein:

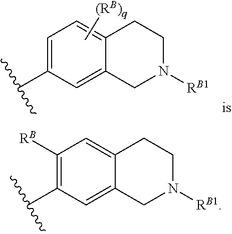

59. The compound of any one of claims 39-58, or a pharmaceutically acceptable salt, solvate, or stereoisomer thereof; wherein:
R^{B1} is hydrogen, —C(═O)R^{a}, C₁-C₆alkyl, or C₁-C₆haloalkyl.

60. The compound of any one of claims 39-59 or a pharmaceutically acceptable salt, solvate, or stereoisomer thereof; wherein:
R^{B1} is hydrogen or C₁-C₆alkyl.

61. The compound of any one of claims 39-60, or a pharmaceutically acceptable salt, solvate, or stereoisomer thereof; wherein:
R^{B1} is hydrogen.

62. The compound of any one of claims 39-61, or a pharmaceutically acceptable salt, solvate, or stereoisomer thereof; wherein:
each R^{B} is independently halogen, —OH, —OR^{a}, —NR^{c}R^{d}, —C(═O)R^{a}, C₁-C₆alkyl, or C₁-C₆haloalkyl; wherein each alkyl are independently substituted with one, two, or three R^{Ba}; or two R^{B} on the same carbon are taken together to form an oxo.

63. The compound of any one of claims 39-62, or a pharmaceutically acceptable salt, solvate, or stereoisomer thereof; wherein:
each R^{B} is independently halogen, —OH, —OR^{a}, —NR^{c}R^{d}, C₁-C₆alkyl, or C₁-C₆haloalkyl; wherein each alkyl are independently substituted with one, two, or three R^{Ba}.

64. The compound of any one of claims 39-63, or a pharmaceutically acceptable salt, solvate, or stereoisomer thereof; wherein:
each R^{B} is independently halogen or C₁-C₆alkyl.

65. The compound of any one of claims 39-64, or a pharmaceutically acceptable salt, solvate, or stereoisomer thereof; wherein:
each R^{B} is independently halogen.

66. The compound of any one of claims 39-65, or a pharmaceutically acceptable salt, solvate, or stereoisomer thereof; wherein:
Ring C is heterocycloalkyl.

67. The compound of any one of claims 39-66, or a pharmaceutically acceptable salt, solvate, or stereoisomer thereof; wherein:
each R^{C} is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl; or two R^{C} on the same carbon are taken together to form an oxo.

68. The compound of any one of claims 39-67, or a pharmaceutically acceptable salt, solvate, or stereoisomer thereof; wherein:
each R^{C} is independently deuterium, halogen, —CN, —OH, —OR^{a}, —NR^{c}R^{d}, C₁-C₆alkyl, or C₁-C₆haloalkyl; or two R^{C} on the same carbon are taken together to form an oxo.

69. The compound of any one of claims 39-68, or a pharmaceutically acceptable salt, solvate, or stereoisomer thereof; wherein:
two R^{C} on the same carbon are taken together to form an oxo.

70. A compound selected from the group consisting of: 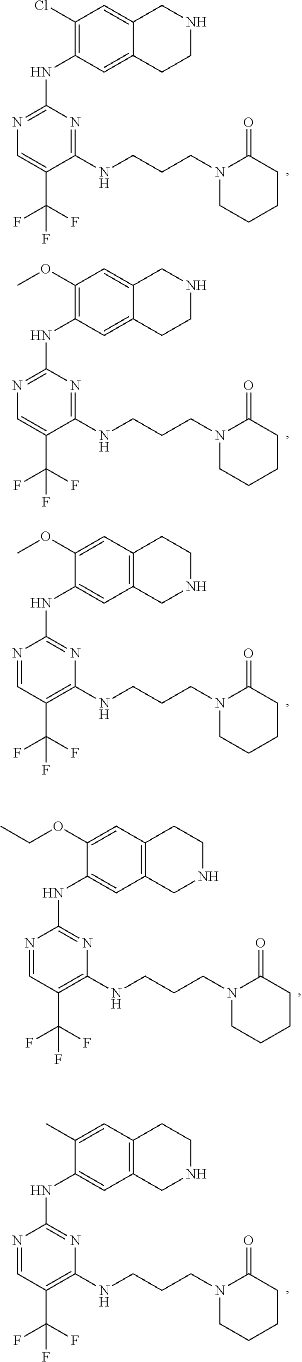 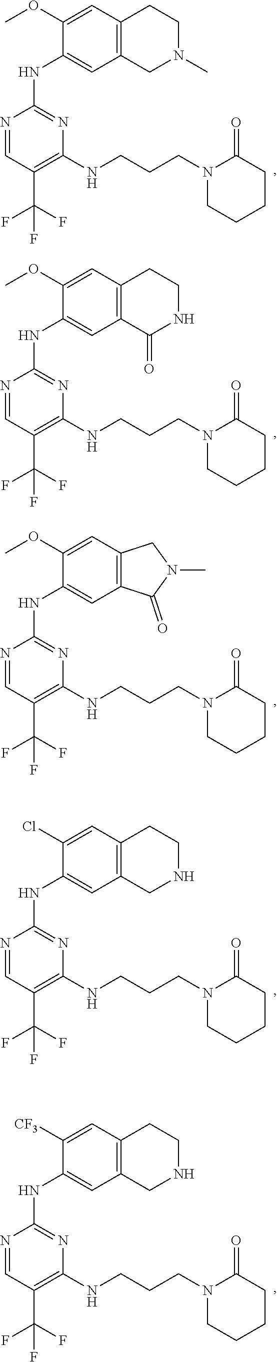 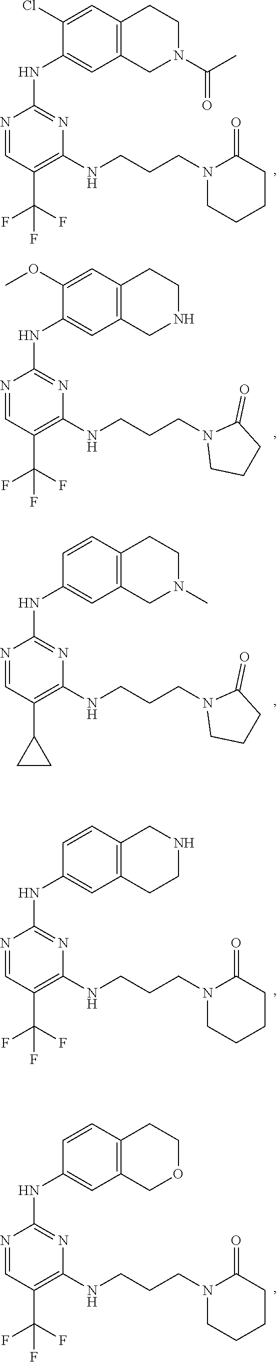 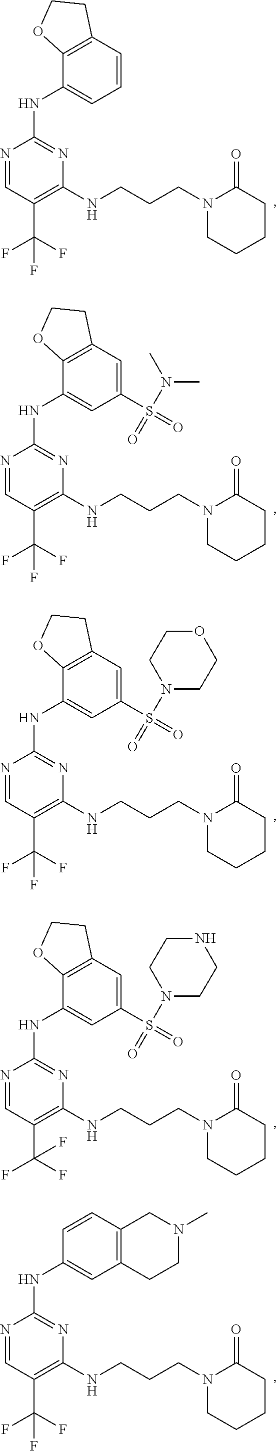 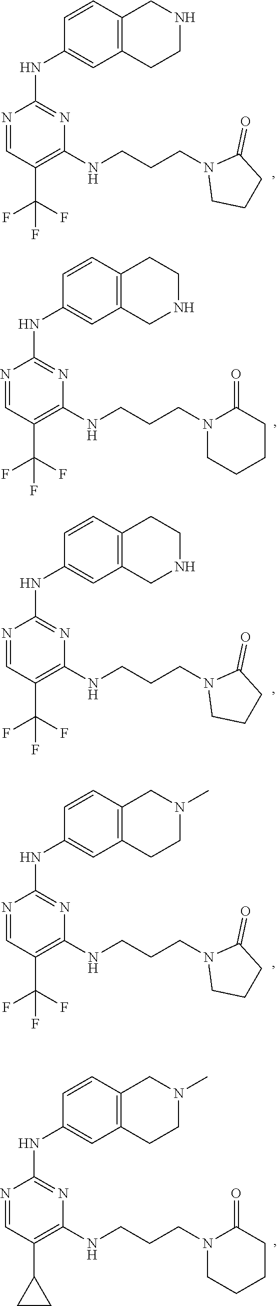 [SMILES: O=C1CCCN1CCCNc1nc(Nc2ccc3c(c2)CCNC3)ncc1C(F)(F)F ; O=C1CCCCN1CCCNc1nc(Nc2ccc3c(c2)CNCC3)ncc1C(F)(F)F ; O=C1CCCN1CCCNc1nc(Nc2ccc3c(c2)CNCC3)ncc1C(F)(F)F ; CN1CCc2cc(Nc3ncc(C(F)(F)F)c(NCCCN4CCCC4=O)n3)ccc2C1 ; CN1CCc2cc(Nc3ncc(C4CC4)c(NCCCN4CCCCC4=O)n3)ccc2C1] 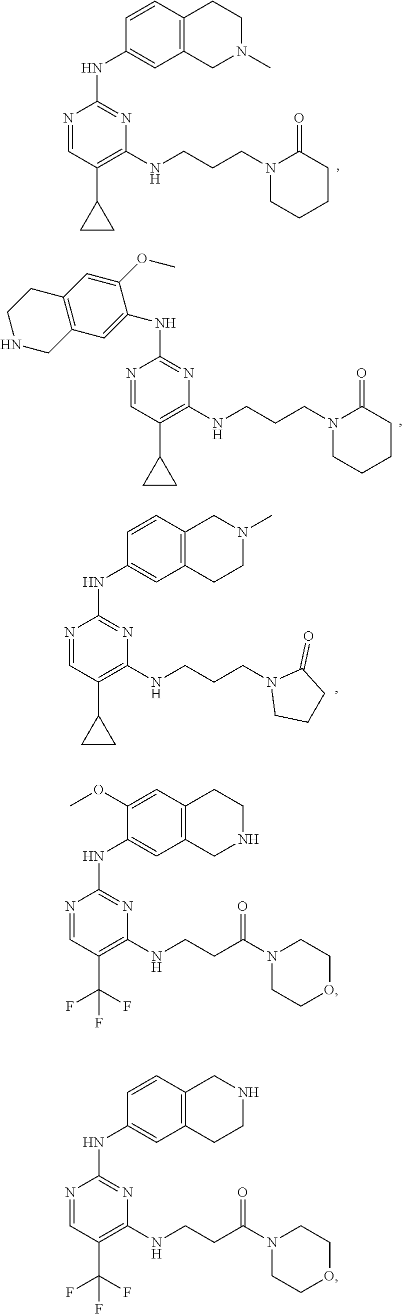 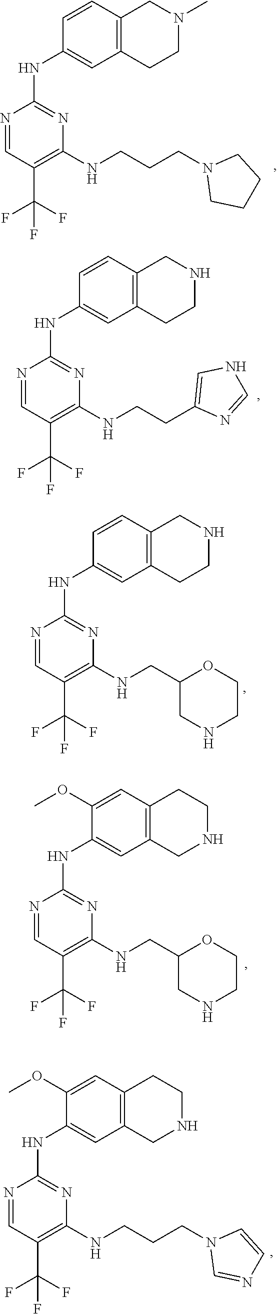 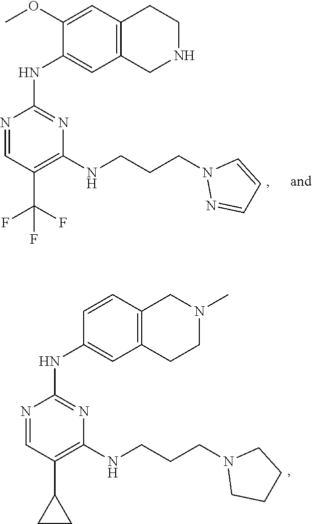 [SMILES: COc1cc2c(cc1Nc1ncc(C(F)(F)F)c(NCCCn3cccn3)n1)CNCC2 ; CN1CCc2cc(Nc3ncc(C4CC4)c(NCCCN4CCCC4)n3)ccc2C1]
or a pharmaceutically acceptable salt, solvate, or stereoisomer thereof.

71. A compound selected from the group consisting of: 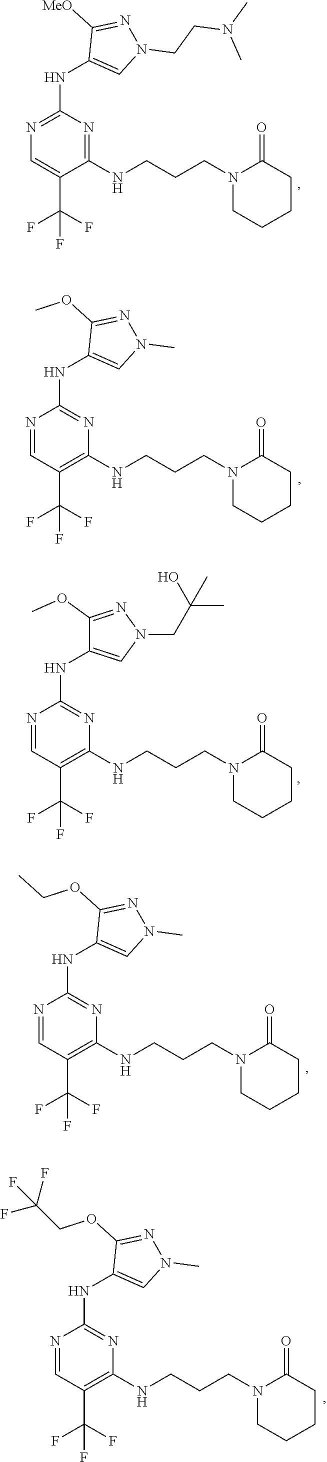 [SMILES: COc1nn(CCN(C)C)cc1Nc1ncc(C(F)(F)F)c(NCCCN2CCCCC2=O)n1 ; COc1nn(C)cc1Nc1ncc(C(F)(F)F)c(NCCCN2CCCCC2=O)n1 ; COc1nn(CC(C)(C)O)cc1Nc1ncc(C(F)(F)F)c(NCCCN2CCCCC2=O)n1 ; CCOc1nn(C)cc1Nc1ncc(C(F)(F)F)c(NCCCN2CCCCC2=O)n1 ; Cn1cc(Nc2ncc(C(F)(F)F)c(NCCCN3CCCCC3=O)n2)c(OCC(F)(F)F)n1] 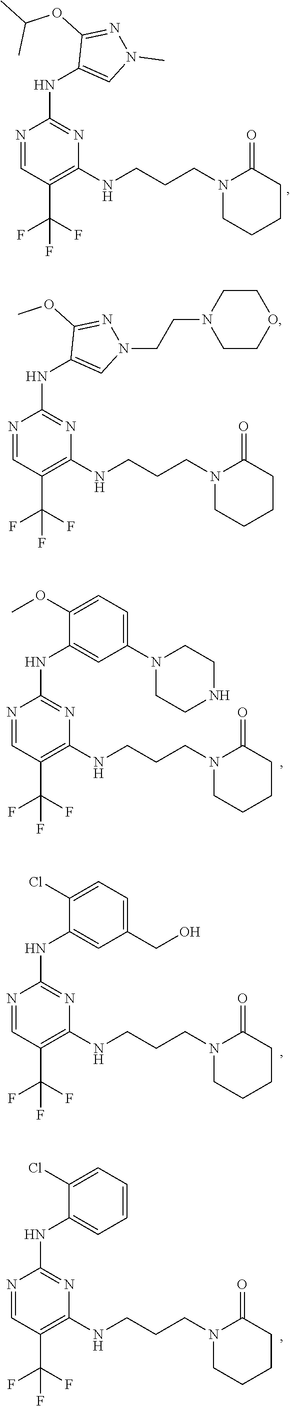 [SMILES: CC(C)Oc1nn(C)cc1Nc1ncc(C(F)(F)F)c(NCCCN2CCCCC2=O)n1 ; COc1nn(CCN2CCOCC2)cc1Nc1ncc(C(F)(F)F)c(NCCCN2CCCCC2=O)n1 ; COc1ccc(N2CCNCC2)cc1Nc1ncc(C(F)(F)F)c(NCCCN2CCCCC2=O)n1 ; O=C1CCCCN1CCCNc1nc(Nc2cc(CO)ccc2Cl)ncc1C(F)(F)F ; O=C1CCCCN1CCCNc1nc(Nc2ccccc2Cl)ncc1C(F)(F)F] 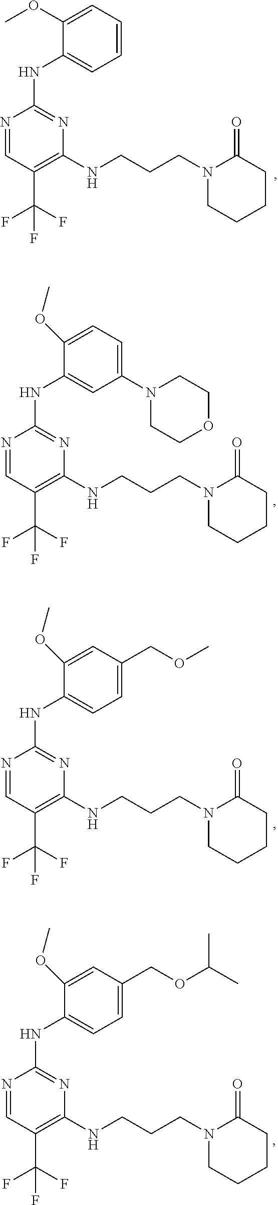 [SMILES: COc1ccccc1Nc1ncc(C(F)(F)F)c(NCCCN2CCCCC2=O)n1 ; COc1ccc(N2CCOCC2)cc1Nc1ncc(C(F)(F)F)c(NCCCN2CCCCC2=O)n1 ; COCc1ccc(Nc2ncc(C(F)(F)F)c(NCCCN3CCCCC3=O)n2)c(OC)c1 ; COc1cc(COC(C)C)ccc1Nc1ncc(C(F)(F)F)c(NCCCN2CCCCC2=O)n1] 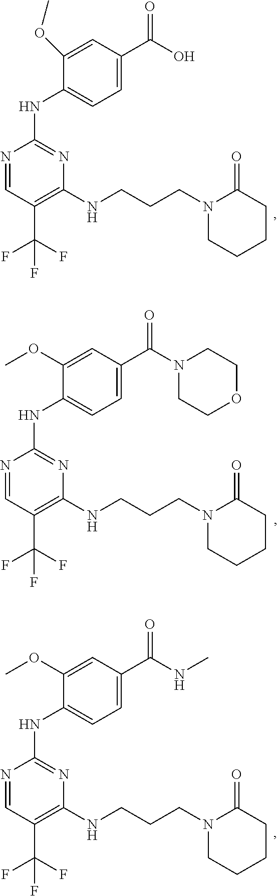 [SMILES: COc1cc(C(=O)O)ccc1Nc1ncc(C(F)(F)F)c(NCCCN2CCCCC2=O)n1 ; COc1cc(C(=O)N2CCOCC2)ccc1Nc1ncc(C(F)(F)F)c(NCCCN2CCCCC2=O)n1 ; CNC(=O)c1ccc(Nc2ncc(C(F)(F)F)c(NCCCN3CCCCC3=O)n2)c(OC)c1] 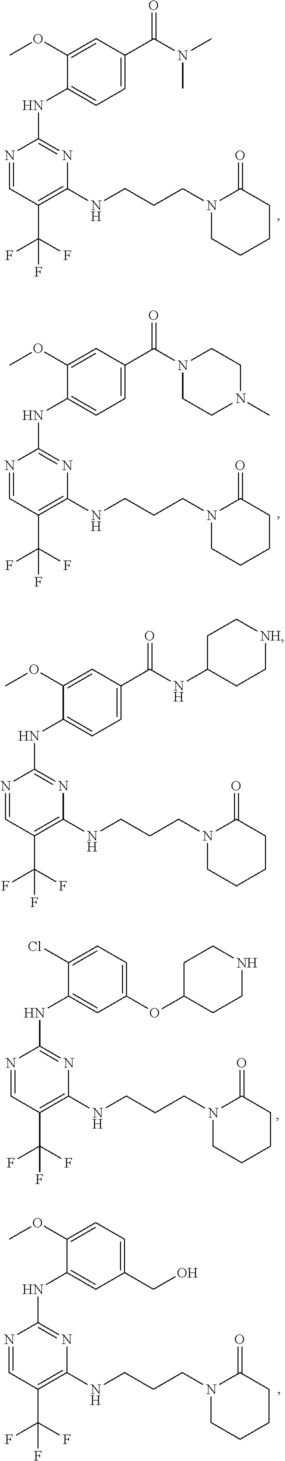 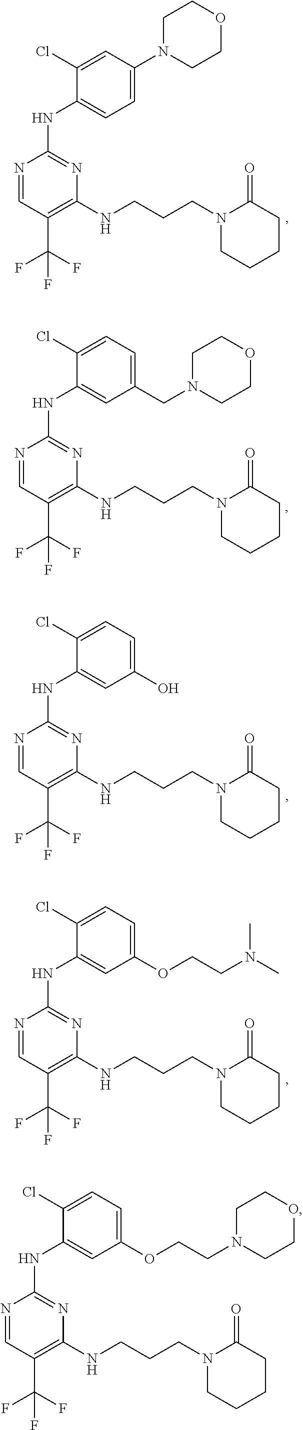 [SMILES: O=C1CCCCN1CCCNc1nc(Nc2ccc(N3CCOCC3)cc2Cl)ncc1C(F)(F)F ; O=C1CCCCN1CCCNc1nc(Nc2cc(CN3CCOCC3)ccc2Cl)ncc1C(F)(F)F ; O=C1CCCCN1CCCNc1nc(Nc2cc(O)ccc2Cl)ncc1C(F)(F)F ; CN(C)CCOc1ccc(Cl)c(Nc2ncc(C(F)(F)F)c(NCCCN3CCCCC3=O)n2)c1 ; O=C1CCCCN1CCCNc1nc(Nc2cc(OCCN3CCOCC3)ccc2Cl)ncc1C(F)(F)F] 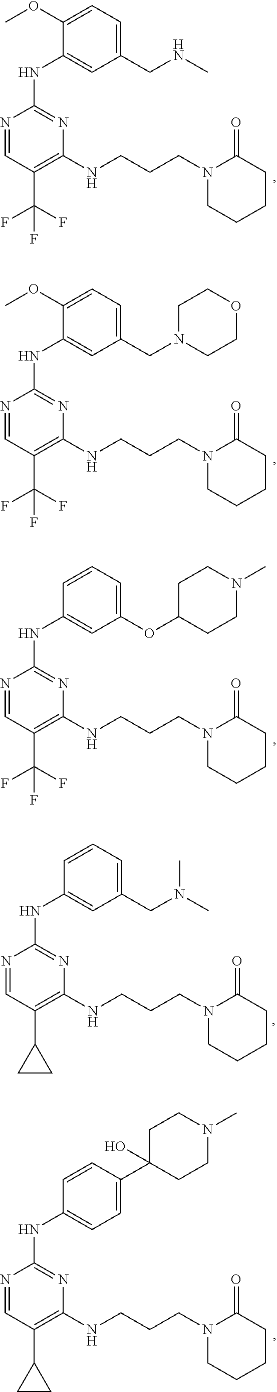 [SMILES: CNCc1ccc(OC)c(Nc2ncc(C(F)(F)F)c(NCCCN3CCCCC3=O)n2)c1 ; COc1ccc(CN2CCOCC2)cc1Nc1ncc(C(F)(F)F)c(NCCCN2CCCCC2=O)n1 ; CN1CCC(Oc2cccc(Nc3ncc(C(F)(F)F)c(NCCCN4CCCCC4=O)n3)c2)CC1 ; CN(C)Cc1cccc(Nc2ncc(C3CC3)c(NCCCN3CCCCC3=O)n2)c1 ; CN1CCC(O)(c2ccc(Nc3ncc(C4CC4)c(NCCCN4CCCCC4=O)n3)cc2)CC1] 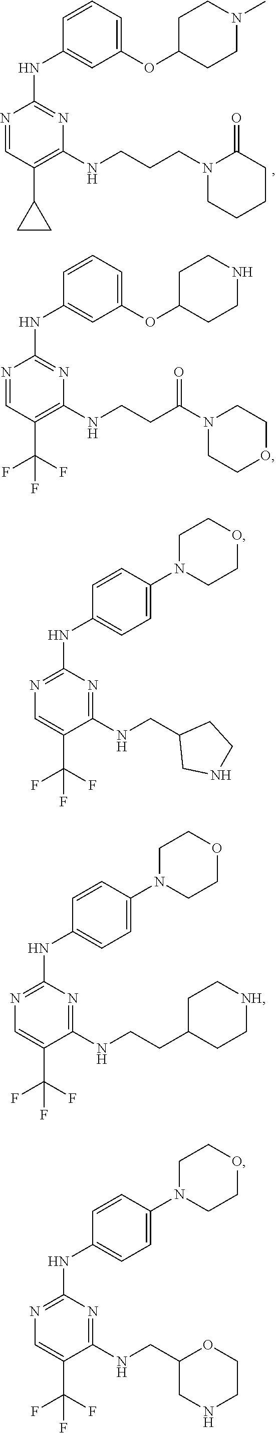 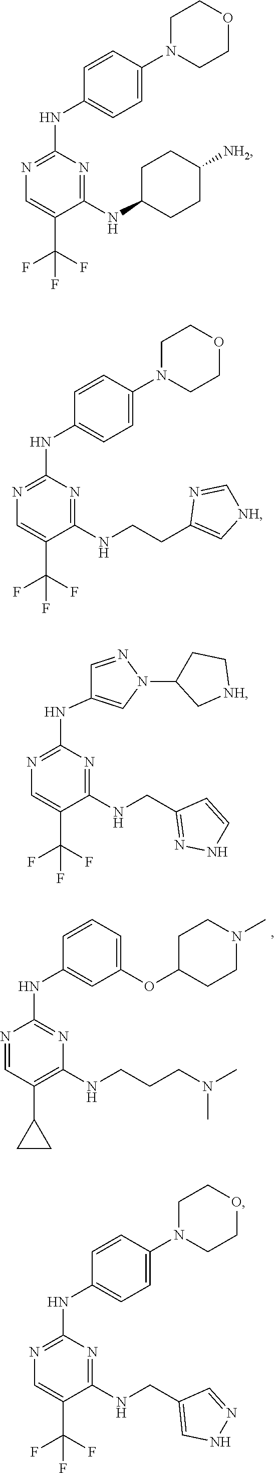 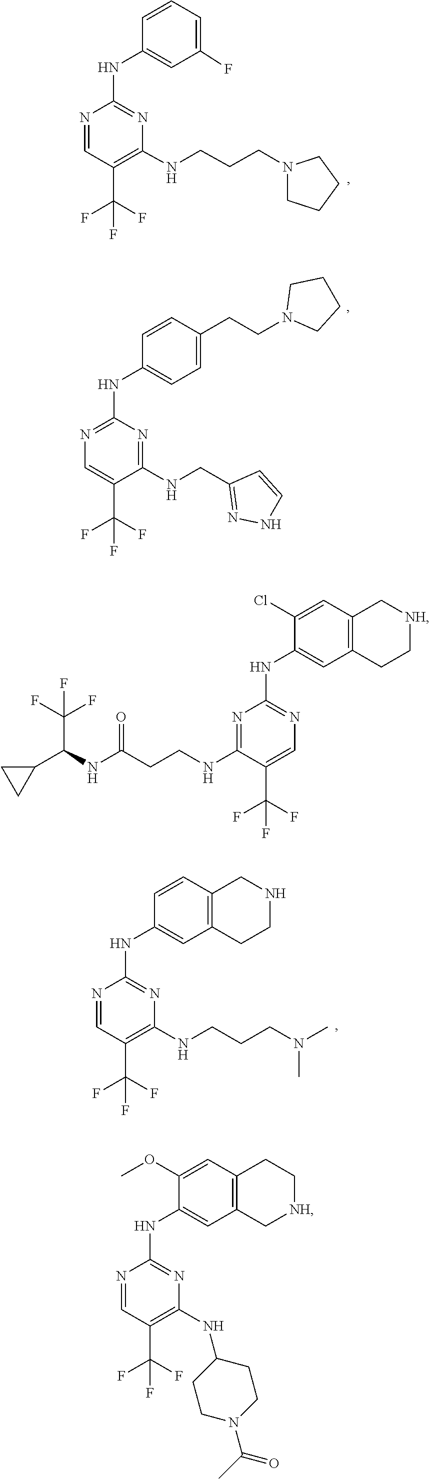 [SMILES: Fc1cccc(Nc2ncc(C(F)(F)F)c(NCCCN3CCCC3)n2)c1 ; FC(F)(F)c1cnc(Nc2ccc(CCN3CCCC3)cc2)nc1NCc1cc[nH]n1 ; O=C(CCNc1nc(Nc2cc3c(cc2Cl)CNCC3)ncc1C(F)(F)F)N[C@@H](C1CC1)C(F)(F)F ; CN(C)CCCNc1nc(Nc2ccc3c(c2)CCNC3)ncc1C(F)(F)F ; COc1cc2c(cc1Nc1ncc(C(F)(F)F)c(NC3CCN(C(C)=O)CC3)n1)CNCC2] 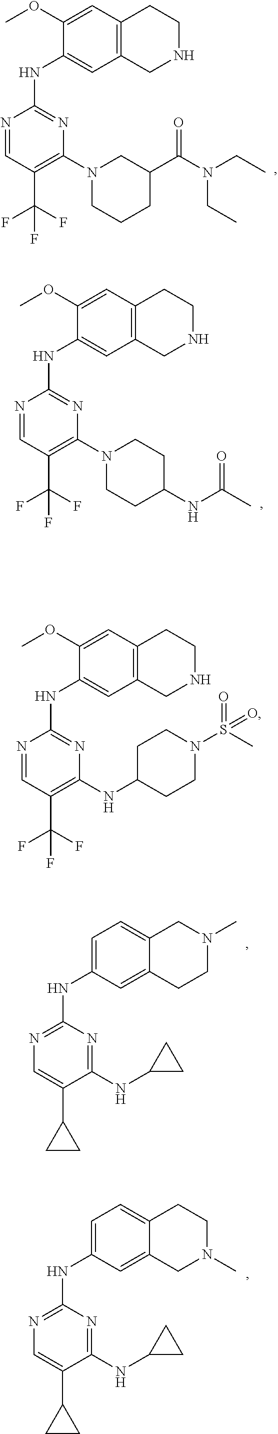 [SMILES: CCN(CC)C(=O)C1CCCN(c2nc(Nc3cc4c(cc3OC)CCNC4)ncc2C(F)(F)F)C1 ; COc1cc2c(cc1Nc1ncc(C(F)(F)F)c(N3CCC(NC(C)=O)CC3)n1)CNCC2 ; COc1cc2c(cc1Nc1ncc(C(F)(F)F)c(NC3CCN(S(C)(=O)=O)CC3)n1)CNCC2 ; CN1CCc2cc(Nc3ncc(C4CC4)c(NC4CC4)n3)ccc2C1 ; CN1CCc2ccc(Nc3ncc(C4CC4)c(NC4CC4)n3)cc2C1] 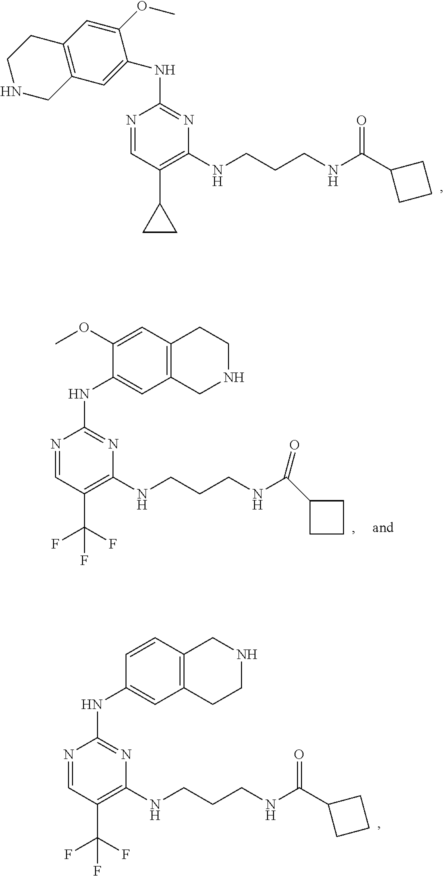 [SMILES: COc1cc2c(cc1Nc1ncc(C3CC3)c(NCCCNC(=O)C3CCC3)n1)CNCC2 ; COc1cc2c(cc1Nc1ncc(C(F)(F)F)c(NCCCNC(=O)C3CCC3)n1)CNCC2 ; O=C(NCCCNc1nc(Nc2ccc3c(c2)CCNC3)ncc1C(F)(F)F)C1CCC1]
or a pharmaceutically acceptable salt, solvate, or stereoisomer thereof.

72. A pharmaceutical composition comprising a compound of any one of claims 1-71, or a pharmaceutically acceptable salt, solvate, or stereoisomer thereof, and a pharmaceutically acceptable excipient.

73. A method of treating cancer in a subject in need thereof, the method comprising administering a compounds of any one of claims 1-71, or a pharmaceutically acceptable salt, solvate, or stereoisomer thereof.

74. The method of claim 73, wherein the cancer is sensitive to ULK1/2 inhibition.

75. The method of claim 73 or 74, wherein the cancer is chronic myeloid leukemia.

76. The method of any one of claims 73-75, wherein the method further comprises administering to the subject in need thereof an additional anti-cancer agent.

77. The method of claim 76, wherein the additional anti-cancer agent is a tyrosine kinase inhibitor.

78. The method of claim 77, wherein the tyrosine kinase inhibitor is imatinib or nilotinib.

79. The method of claim 76, wherein the additional anti-cancer treatment is radiotherapy.
